(19) United States
(12) Patent Application Publication
(10) Pub. No.: US 2003/0148305 A1
(43) Pub. Date: Aug. 7, 2003

(54) Nucleic acids, polypeptides, single nucleotide polymorphisms and methods of use thereof

(76) Inventors: William M. Grosse, Branford, CT (US); John P. Alsobrook II, Madison, CT (US); Denise M. Lepley, Branford, CT (US); Catherine E. Burgess, Wethersfield, CT (US); Joel S. Bader, Stamford, CT (US); Aruna Bansal, Landbeach (GB); Carol E.A. Pena, New Haven, CT (US); Richard A. Shimkets, Guilford, CT (US); Ramesh Kekuda, Norwalk, CT (US); Bryan D. Zerhusen, Branford, CT (US); Glennda Smithson, Guilford, CT (US); David W. Anderson, Branford, CT (US); Mei Zhong, Branford, CT (US); Charles E. Miller, Guilford, CT (US); Corine A.M. Vernet, Branford, CT (US); Tord Hjalt, Lomma (SE)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/177,809

(22) Filed: Jun. 21, 2002

Related U.S. Application Data
(60) Provisional application No. 60/299,949, filed on Jun. 21, 2001.
(60) Provisional application No. 60/300,290, filed on Jun. 22, 2001.
(60) Provisional application No. 60/311,285, filed on Aug. 9, 2001.
(60) Provisional application No. 60/327,892, filed on Oct. 9, 2001.
(60) Provisional application No. 60/327,345, filed on Oct. 5, 2001.

(51) Int. Cl. C12Q 1/68; G01N 33/53; C07H 21/04; C12P 21/02; C12N 5/06; C07K 14/47; A61K 38/17

(52) U.S. Cl. 435/6; 435/7.1; 435/69.1; 435/320.1; 435/325; 514/12; 530/350; 536/23.5

(57) ABSTRACT

Disclosed herein is a nucleic acid sequence that encodes a novel polypeptide. Also disclosed is a polypeptide encoded by the nucleic acid sequence, and antibodies, which immunospecifically-bind to the polypeptide, as well as derivatives, variants, mutants, or fragments of the aforementioned polypeptide, polynucleotide, or antibody. The invention further discloses therapeutic, diagnostic and research methods for diagnosis, treatment, and prevention of disorders involving this novel human nucleic acid and protein. The invention also provides nucleic acids containing single-nucleotide polymorphisms identified for transcribed human sequences, as well as methods of using the nucleic acids.

### RELATED APPLICATIONS

This application claims priority to U.S. S. No. 60/299,949, filed Jun. 21, 2001; U.S. S. No. 60/300,290, filed Jun. 22, 2001; U.S. S. No. 60/311,285, filed on Aug. 9, 2001; U.S. S. No. 60/327,892, filed Oct. 9, 2001; U.S. S. No. 60/327,345, filed on Oct. 5, 2001, each of which is incorporated herein by reference in its entirety.

### BACKGROUND OF THE INVENTION

The present invention is also based in part on nucleic acids encoding proteins that are new members of the hexokinase III-like family. More particularly, the invention relates to nucleic acids encoding novel polypeptides, as well as vectors, host cells, antibodies, and recombinant methods for producing these nucleic acids and polypeptides.

This invention also relates to sequence polymorphisms. Sequence polymorphism-based analysis of nucleic acid sequences can augment or replace previously known methods for determining the identity and relatedness of individuals. The approach is generally based on alterations in nucleic acid sequences between related individuals. This analysis has been widely used in a variety of genetic, diagnostic, and forensic applications. For example, polymorphism analyses are used in identity and paternity analyses, and in genetic mapping studies.

One such type of variation is a restriction fragment length polymorphism (RFLP). RFLPS can create or delete a recognition sequence for a restriction endonuclease in one nucleic acid relative to a second nucleic acid. The result of the variation is an alteration in the relative length of restriction enzyme generated DNA fragments in the two nucleic acids.

Other polymorphisms take the form of short tandem repeats (STR) sequences, which are also referred to as variable numbers of tandem repeat (VNTR) sequences. STR sequences typically include tandem repeats of 2, 3, or 4 nucleotide sequences that are present in a nucleic acid from one individual but absent from a second, related individual at the corresponding genomic location.

Other polymorphisms take the form of single nucleotide variations, termed single nucleotide polymorphisms (SNPs), between individuals. A SNP can, in some instances, be referred to as a “cSNP” to denote that the nucleotide sequence containing the SNP originates as a cDNA.

SNPs can arise in several ways. A single nucleotide polymorphism may arise due to a substitution of one nucleotide for another at the polymorphic site. Substitutions can be transitions or transversions. A transition is the replacement of one purine nucleotide by another purine nucleotide, or one pyrimidine by another pyrimidine. A transversion is the replacement of a purine by a pyrimidine, or the converse.

Single nucleotide polymorphisms can also arise from a deletion of a nucleotide or an insertion of a nucleotide relative to a reference allele. Thus, the polymorphic site is a site at which one allele bears a gap with respect to a single nucleotide in another allele. Some SNPs occur within, or near genes. One such class includes SNPs falling within regions of genes encoding for a polypeptide product. These SNPs may result in an alteration of the amino acid sequence of the polypeptide product and give rise to the expression of a defective or other variant protein. Such variant products can, in some cases result in a pathological condition, e.g., genetic disease. Examples of genes in which a polymorphism within a coding sequence gives rise to genetic disease include sickle cell anemia and cystic fibrosis. Other SNPs do not result in alteration of the polypeptide product. Of course, SNPs can also occur in noncoding regions of genes.

SNPs tend to occur with great frequency and are spaced uniformly throughout the genome. The frequency and uniformity of SNPs means that there is a greater probability that such a polymorphism will be found in close proximity to a genetic locus of interest.

### SUMMARY OF THE INVENTION

The invention is based in part upon the discovery of nucleic acid sequences encoding novel polypeptides. The novel nucleic acid and polypeptide, as well as derivatives, homologs, analogs and fragments thereof, will hereinafter be designated as NOV1 nucleic acid or polypeptide sequences.

In one aspect, the invention provides an isolated NOV1 nucleic acid molecule encoding a NOV1 polypeptide that includes a nucleic acid sequence that has identity to the nucleic acid disclosed in SEQ ID NO: 1. In some embodiments, the NOV1 nucleic acid molecule will hybridize under stringent conditions to a nucleic acid sequence complementary to a nucleic acid molecule that includes a protein-coding sequence of a NOV1 nucleic acid sequence. The invention also includes an isolated nucleic acid that encodes a NOV1 polypeptide, or a fragment, homolog, analog or derivative thereof. For example, the nucleic acid can encode a polypeptide at least 95% identical to a polypeptide comprising the amino acid sequences of SEQ ID NO:2. The nucleic acid can be, for example, a genomic DNA fragment or a cDNA molecule that includes the nucleic acid sequence of any of SEQ ID NO: 1. In one embodiment, the nucleic acid and polypeptide are naturally occcurring.

Also included in the invention is an oligonucleotide, e.g., an oligonucleotide which includes at least 6 contiguous nucleotides of a NOV1 nucleic acid (e.g., SEQ ID NO: 1) or a complement of said oligonucleotide. Also included in the invention are substantially purified NOV1 polypeptides (SEQ ID NO:2). In certain embodiments, the NOV1 polypeptides include an amino acid sequence that is substantially identical to the amino acid sequence of a human NOV1 polypeptide.

The invention also features antibodies that immunoselectively bind to NOV1 polypeptides, or fragments, homologs, analogs or derivatives thereof. The antibody could be a monoclonal antibody, a humanized antibody or a fully human antibody. In one embodiment, the the dissociation constant for the binding of the polypeptide to the antibody is less than 1×10⁻⁹ M. In another embodiment, the antibody could neutralize an activity of the polypeptide.

In another aspect, the invention includes pharmaceutical compositions that include therapeutically- or prophylactically-effective amounts of a therapeutic and a pharmaceutically-acceptable carrier. The therapeutic can be, e.g., a NOV1 nucleic acid, a NOV1 polypeptide, or an antibody specific for a NOV1 polypeptide. In a further aspect, the invention includes, in one or more containers, a therapeutically- or prophylactically-effective amount of this pharmaceutical composition.

In a further aspect, the invention includes a method of producing a polypeptide by culturing a cell that includes a NOV1 nucleic acid, under conditions allowing for expression of the NOV1 polypeptide encoded by the DNA. If desired, the NOV1 polypeptide can then be recovered. The invention also includes a kit comprising the polypeptide.

In another aspect, the invention includes a method of detecting the presence of a NOV1 polypeptide in a sample. In the method, a sample is contacted with a compound that selectively binds to the polypeptide under conditions allowing for formation of a complex between the polypeptide and the compound. The complex is detected, if present, thereby identifying the NOV1 polypeptide within the sample.

The invention also includes methods to identify specific cell or tissue types based on their expression of a NOV1. In a preferred embodiment, the cell is bacterial, mammalian, insect or yeast. The invention also includes a method of producing the NOV1 polypeptides, the method comprising culturing a cell under conditions that lead to expression of the polypeptide, wherein the cell comprises a vector with an isolated NOV1 nucleic acid molecule.

Also included in the invention is a method of detecting the presence of a NOV1 nucleic acid molecule in a sample by contacting the sample with a NOV1 nucleic acid probe or primer, and detecting whether the nucleic acid probe or primer bound to a NOV1 nucleic acid molecule in the sample.

In a further aspect, the invention provides a method for modulating the activity of a NOV1 polypeptide by contacting a cell sample that includes the NOV1 polypeptide with a compound that binds to the NOV1 polypeptide in an amount sufficient to modulate the activity of said polypeptide. The compound can be, e.g., a small molecule, such as a nucleic acid, peptide, polypeptide, peptidomimetic, carbohydrate, lipid or other organic (carbon containing) or inorganic molecule, as further described herein.

Also within the scope of the invention is the use of a therapeutic in the manufacture of a medicament for treating or preventing disorders or syndromes including, e.g., metastatic melanoma, Von Hippel-Lindau (VHL) syndrome, cirrhosis, transplantation, systemic lupus erythematosus, autoimmune disease, asthma, emphysema, scleroderma, allergy, ARDS, endometriosis, fertility, anemia, ataxia-telangiectasia, autoimmume disease, immunodeficiencies, lymphedema, allergies, obesity, high blood pressure, diabetes, hemophilia, hypercoagulation, idiopathic thrombocytopenic purpura, immunodeficiencies, graft vesus host, cancer, trauma, regeneration (in vitro and in vivo), viral/bacterial/parasitic infections, such as Huntington's disease and/or other pathologies and disorders of the like.

The therapeutic can be, e.g., a NOV1 nucleic acid, a NOV1 polypeptide, or a NOV1-specific antibody, or biologically-active derivatives or fragments thereof.

For example, the compositions of the present invention will have efficacy for treatment of patients suffering from the diseases and disorders disclosed above and/or other pathologies and disorders of the like. The polypeptides can be used as immunogens to produce antibodies specific for the invention, and as vaccines. They can also be used to screen for potential agonist and antagonist compounds. For example, a cDNA encoding NOV1 may be useful in gene therapy, and NOV1 may be useful when administered to a subject in need thereof. By way of non-limiting example, the compositions of the present invention will have efficacy for treatment of patients suffering from the diseases and disorders disclosed above and/or other pathologies and disorders of the like.

The invention also includes a method for determining the presence or amount of the the NOV1 polypeptide, the method comprising: (a) providing said sample; (b) introducing said sample to an antibody that binds immunospecifically to the polypeptide; and (c) determining the presence or amount of antibody bound to said polypeptide, thereby determining the presence or amount of polypeptide in said sample. The invention also provides a method for determining the presence of or predisposition to a disease associated with altered levels of expression of the NOV1 polypeptide in a first mammalian subject, the method comprising: (a) measuring the level of expression of the polypeptide in a sample from the first mammalian subject; and (b) comparing the expression of said polypeptide in the sample of step (a) to the expression of the polypeptide present in a control sample from a second mammalian subject known not to have, or not to be predisposed to, said disease, wherein an alteration in the level of expression of the polypeptide in the first subject as compared to the control sample indicates the presence of or predisposition to said disease.

The invention further includes a method for screening for a modulator of disorders or syndromes including, e.g., the diseases and disorders disclosed above and/or other pathologies and disorders of the like. The method includes contacting a test compound with a NOV1 polypeptide and determining if the test compound binds to said NOV1 polypeptide. Binding of the test compound to the NOV1 polypeptide indicates the test compound is a modulator of activity, or of latency or predisposition to the aforementioned disorders or syndromes. Also within the scope of the invention is a method for screening for a modulator of activity, or of latency or predisposition to disorders or syndromes including, e.g., the diseases and disorders disclosed above and/or other pathologies and disorders of the like by administering a test compound to a test animal at increased risk for the aforementioned disorders or syndromes. The test animal expresses a recombinant polypeptide encoded by a NOV1 nucleic acid. Expression or activity of NOV1 polypeptide is then measured in the test animal, as is expression or activity of the protein in a control animal which recombinantly-expresses NOV1 polypeptide and is not at increased risk for the disorder or syndrome. Next, the expression of NOV1 polypeptide in both the test animal and the control animal is compared. A change in the activity of NOV1 polypeptide in the test animal relative to the control animal indicates the test compound is a modulator of latency of the disorder or syndrome.

In another aspect, the invention also includes a method for determining the presence of or predisposition to a disease associated with altered levels of expression of the NOVX nucleic acid molecule in a first mammalian subject, the method comprising: measuring the level of expression of the nucleic acid in a sample from the first mammalian subject; and (a) comparing the level of expression of said nucleic acid in the sample of step (a) to the level of expression of the nucleic acid present in a control sample from a second mammalian subject known not to have or not be predisposed to, the disease; wherein an alteration in the level of expression of the nucleic acid in the first subject as compared to the control sample indicates the presence of or predisposition to the disease.

The invention also provides a method for modulating the activity of the polypeptide of claim 1, the method comprising contacting a cell sample expressing the polypeptide of claim 1 with a compound that binds to said polypeptide in an amount sufficient to modulate the activity of the polypeptide.

In another aspect, the invention provides a method of treating or preventing a pathology associated with the polypeptide of claim 1, the method comprising administering the NOVX polypeptide to a subject in which such treatment or prevention is desired in an amount sufficient to treat or prevent the pathology in the subject. The invention also includes a method of treating a pathological state in a mammal, the method comprising administering to the mammal a polypeptide or an antibody to the polypeptide in an amount that is sufficient to alleviate the pathological state, wherein the polypeptide is a polypeptide having an amino acid sequence at least 95% identical to a polypeptide comprising the amino acid sequence of SEQ ID NO:2 or a biologically active fragment thereof.

The invention also provides a method of identifying an agent that binds to the NOV1 polypeptide, the method comprising:(a) introducing said polypeptide to said agent; and (b) determining whether said agent binds to said polypeptide. In yet another aspect, the invention includes a method for determining the presence of or predisposition to a disease associated with altered levels of a NOV1 polypeptide, a NOV1 nucleic acid, or both, in a subject (e.g., a human subject). The method includes measuring the amount of the NOV1 polypeptide in a test sample from the subject and comparing the amount of the polypeptide in the test sample to the amount of the NOV1 polypeptide present in a control sample. An alteration in the level of the NOV1 polypeptide in the test sample as compared to the control sample indicates the presence of or predisposition to a disease in the subject. Preferably, the predisposition includes, e.g., the diseases and disorders disclosed above and/or other pathologies and disorders of the like. Also, the expression levels of the new polypeptides of the invention can be used in a method to screen for various cancers as well as to determine the stage of cancers.

In a further aspect, the invention includes a method of treating or preventing a pathological condition associated with a disorder in a mammal by administering to the subject a NOV1 polypeptide, a NOV1 nucleic acid, or a NOV1-specific antibody to a subject (e.g., a human subject), in an amount sufficient to alleviate or prevent the pathological condition. In preferred embodiments, the disorder, includes, e.g., the diseases and disorders disclosed above and/or other pathologies and disorders of the like.

In yet another aspect, the invention can be used in a method to identity the cellular receptors and downstream effectors of the invention by any one of a number of techniques commonly employed in the art. These include but are not limited to the two-hybrid system, affinity purification, co-precipitation with antibodies or other specific-interacting molecules. NOV1 nucleic acids and polypeptides are further useful in the generation of antibodies that bind immuno-specifically to the novel NOV1 substances for use in therapeutic or diagnostic methods. These NOV1 antibodies may be generated according to methods known in the art, using prediction from hydrophobicity charts. The disclosed NOV1 proteins have multiple hydrophilic regions, each of which can be used as an immunogen. These NOV1 proteins can be used in assay systems for functional analysis of various human disorders, which will help in understanding of pathology of the disease and development of new drug targets for various disorders.

The NOV1 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here. The invention also includes a vector comprising the NOV1 nucleic acid molecule. In a preferred embodiment, the vector further comprises promoter operably linked to said nucleic acid molecule.

The invention is also based in part on the discovery of novel single nucleotide polymorphisms (SNPs) in regions of human DNA. Accordingly, in one aspect, the invention provides an isolated polynucleotide which includes one or more of the SNPs described herein. The polynucleotide can be, e.g., a nucleotide sequence which includes one or more of the polymorphic sequences shown in Tables 5-7 (SEQ ID NOs:5, 8 and 11) and which includes a polymorphic sequence, or a fragment of the polymorphic sequence, as long as it includes the polymorphic site. The polynucleotide may alternatively contain a nucleotide sequence which includes a sequence complementary to one or more of the sequences, or a fragment of the complementary nucleotide sequence, provided that the fragment includes a polymorphic site in the polymorphic sequence. The invention also provides an isolated nucleic acid comprising the 5′ untranslated region of SEQ ID NO:3, 5, 6, 8, 9, or 11.

The polynucleotide can be, e.g., DNA or RNA, and can be between about 10 and about 100 nucleotides, e.g, 10-90, 10-75, 10-51, 10-40, or 10-30, nucleotides in length.

In some embodiments, the polymorphic site in the polymorphic sequence includes a nucleotide other than the nucleotide (e.g., base change) listed in Tables 5-7 for the polymorphic sequence.

In other embodiments, the complement of the polymorphic site includes a nucleotide other than the complement of the nucleotide listed in Tables 5-7 for the complement of the polymorphic sequence, e.g., the complement of the nucleotide listed in Tables 5-7 for the polymorphic sequence. In some embodiments, the polymorphic sequence is associated with a polypeptide related to one of the protein families disclosed herein.

In another aspect, the invention provides an isolated allele-specific oligonucleotide that hybridizes to a first polynucleotide containing a polymorphic site. The first polynucleotide can be, e.g., a nucleotide sequence comprising one or more polymorphic sequences, provided that the polymorphic sequence includes a nucleotide other than the nucleotide recited Tables 5-7 for the polymorphic sequence. Alternatively, the first polynucleotide can be a nucleotide sequence that is a fragment of the polymorphic sequence, provided that the fragment includes a polymorphic site in the polymorphic sequence, or a complementary nucleotide sequence which includes a sequence complementary to one or more polymorphic sequences, provided that the complementary nucleotide sequence includes a nucleotide other than the complement of the nucleotide recited in Tables 5-7. The first polynucleotide may in addition include a nucleotide sequence that is a fragment of the complementary sequence, provided that the fragment includes a polymorphic site in the polymorphic sequence.

In some embodiments, the oligonucleotide does not hybridize under stringent conditions to a second polynucleotide. The second polynucleotide can be, e.g., (a) a nucleotide sequence comprising one or more polymorphic sequences, wherein the polymorphic sequence includes the nucleotide listed in Tables 5-7 for the polymorphic sequence; (b) a nucleotide sequence that is a fragment of any of the polymorphic sequences; (c) a complementary nucleotide sequence including a sequence complementary to one or more polymorphic sequences, wherein the polymorphic sequence includes the complement of the nucleotide listed in Tables 5-7; and (d) a nucleotide sequence that is a fragment of the complementary sequence, provided that the fragment includes a polymorphic site in the polymorphic sequence.

The oligonucleotide can be, e.g., between about 10 and about 100 bases in length. In some embodiments, the oligonucleotide is between about 10 and 75 bases, 10 and 51 bases, 10 and about 40 bases, or about 15 and 30 bases in length.

The invention also provides a method of detecting a polymorphic site in a nucleic acid. The method includes contacting the nucleic acid with an oligonucleotide that hybridizes to a polymorphic sequence selected from the group consisting of SEQ ID NO:5, 8 and 11, or its complement, provided that the polymorphic sequence includes a nucleotide other than the nucleotide recited in Tables 5-7 for the polymorphic sequence, or the complement includes a nucleotide other than the complement of the nucleotide recited in Tables 5-7 The method also includes determining whether the nucleic acid and the oligonucleotide hybridize. Hybridization of the oligonucleotide to the nucleic acid sequence indicates the presence of the polymorphic site in the nucleic acid.

In preferred embodiments, the oligonucleotide does not hybridize to the polymorphic sequence when the polymorphic sequence includes the nucleotide recited in Tables 5-7 for the polymorphic sequence, or when the complement of the polymorphic sequence includes the complement of the nucleotide recited in Tables 5-7 for the polymorphic sequence.

The oligonucleotide can be, e.g., between about 10 and about 100 bases in length. In some embodiments, the oligonucleotide is between about 10 and 75 bases, 10 and 51 bases, 10 and about 40 bases, or about 15 and 30 bases in length.

In some embodiments, the polymorphic sequence identified by the oligonucleotide is associated with a polypeptide related to one of the protein families disclosed herein. For example, the nucleic acid may be an associated polypeptide related to a hexokinase 3, SIAT1, or PEX6 protein.

In another aspect, the method includes determining if a sequence polymorphism is present in a subject, such as a human. The method includes providing a nucleic acid from the subject and contacting the nucleic acid with an oligonucleotide that hybridizes to a polymorphic sequence selected from the group consisting of SEQ ID NOS:5, 8 and 11, or its complement, provided that the polymorphic sequence includes a nucleotide other than the nucleotide recited in Tables 5-7 for said polymorphic sequence, or the complement includes a nucleotide other than the complement of the nucleotide recited in Tables 5-7. Hybridization between the nucleic acid and the oligonucleotide is then determined. Hybridization of the oligonucleotide to the nucleic acid sequence indicates the presence of the polymorphism in said subject.

In a further aspect, the invention provides a method of determining the relatedness of a first and second nucleic acid. The method includes providing a first nucleic acid and a second nucleic acid and contacting the first nucleic acid and the second nucleic acid with an oligonucleotide or primer that hybridizes to a polymorphic sequence selected from the group consisting of SEQ ID NOs:5, 8 and 11, or its complement, provided that the polymorphic sequence includes a nucleotide other than the nucleotide recited in Tables 5-7 for the polymorphic sequence, or the complement includes a nucleotide other than the complement of the nucleotide recited in Tables 5-7. In a preferred embodiment, the oligonucleotide is between 17-35 nucleotides. The method also includes determining whether the first nucleic acid and the second nucleic acid hybridize to the oligonucleotide, and comparing hybridization of the first and second nucleic acids to the oligonucleotide. Hybridization of first and second nucleic acids to the nucleic acid indicates the first and second subjects are related.

In preferred embodiments, the oligonucleotide does not hybridize to the polymorphic sequence when the polymorphic sequence includes the nucleotide recited in Tables 5-7 for the polymorphic sequence, or when the complement of the polymorphic sequence includes the complement of the nucleotide recited in Tables 5-7 for the polymorphic sequence.

The oligonucleotide can be, e.g., between about 10 and about 100 bases in length. In some embodiments, the oligonucleotide is between about 10 and 75 bases, 10 and 51 bases, 10 and about 40 bases, or about 15 and 30 bases in length.

The method can be used in a variety of applications. For example, the first nucleic acid may be isolated from physical evidence gathered at a crime scene, and the second nucleic acid may be obtained from a person suspected of having committed the crime. Matching the two nucleic acids using the method can establish whether the physical evidence originated from the person.

In another example, the first sample may be from a human male suspected of being the father of a child and the second sample may be from the child. Establishing a match using the described method can establish whether the male is the father of the child.

In another aspect, the invention provides an isolated polypeptide comprising a polymorphic site at one or more amino acid residues, and wherein the protein is encoded by a polynucleotide including one of the polymorphic sequences SEQ ID NOs:5, 8 and 11, or their complement, provided that the polymorphic sequence includes a nucleotide other than the nucleotide recited in Tables 5-7 for the polymorphic sequence, or the complement includes a nucleotide other than the complement of the nucleotide recited in Tables 5-7.

The polypeptide can be, e.g., related to one of the protein families disclosed herein. For example, the polypeptide can be related to a hexokinase 3, SIAT1 or PEX6 protein in Tables 5-7.

In some embodiments, the polypeptide is translated in the same open reading frame as is a wild type protein whose amino acid sequence is identical to the amino acid sequence of the polymorphic protein except at the site of the polymorphism.

In some embodiments, the polypeptide encoded by the polymorphic sequence, or its complement, includes the nucleotide listed in Tables 5-7 for the polymorphic sequence, or the complement includes the complement of the nucleotide listed in Tables 5-7.

The invention also provides an antibody that binds specifically to a polypeptide encoded by a polynucleotide comprising a nucleotide sequence encoded by a polynucleotide selected from the group consisting of polymorphic sequences SEQ ID NOS:5, 8 and 11, or its complement. The polymorphic sequence includes a nucleotide other than the nucleotide recited in Tables 5-7 for the polymorphic sequence, or the complement includes a nucleotide other than the complement of the nucleotide recited in Tables 5-7.

In some embodiments, the antibody binds specifically to a polypeptide encoded by a polymorphic sequence which includes the nucleotide listed in Tables 5-7 for the polymorphic sequence.

Preferably, the antibody does not bind specifically to a polypeptide encoded by a polymorphic sequence which includes the nucleotide listed in Tables 5-7 for the polymorphic sequence.

The invention further provides a method of detecting the presence of a polypeptide having one or more amino acid residue polymorphisms in a subject. The method includes providing a protein sample from the subject and contacting the sample with the above-described antibody under conditions that allow for the formation of antibody-antigen complexes. The antibody-antigen complexes are then detected. The presence of the complexes indicates the presence of the polypeptide.

The invention also provides a method of treating a subject suffering from, at risk for, or suspected of, suffering from a pathology ascribed to the presence of a sequence polymorphism in a subject, e.g., a human, non-human primate, cat, dog, rat, mouse, cow, pig, goat, or rabbit. The method includes providing a subject suffering from a pathology associated with aberrant expression of a first nucleic acid comprising a polymorphic sequence selected from the group consisting of SEQ ID NOS:5, 8 and 11, or its complement, and treating the subject by administering to the subject an effective dose of a therapeutic agent. Aberrant expression can include qualitative alterations in expression of a gene, e.g., expression of a gene encoding a polypeptide having an altered amino acid sequence with respect to its wild-type counterpart. Qualitatively different polypeptides can include, shorter, longer, or altered polypeptides relative to the amino acid sequence of the wild-type polypeptide. Aberrant expression can also include quantitative alterations in expression of a gene. Examples of quantitative alterations in gene expression include lower or higher levels of expression of the gene relative to its wild-type counterpart, or alterations in the temporal or tissue-specific expression pattern of a gene. Finally, aberrant expression may also include a combination of qualitative and quantitative alterations in gene expression.

The therapeutic agent can include, e.g., second nucleic acid comprising the polymorphic sequence, provided that the second nucleic acid comprises the nucleotide present in the wild type allele. In some embodiments, the second nucleic acid sequence comprises a polymorphic sequence which includes nucleotide listed in Tables 5-7 for the polymorphic sequence.

Alternatively, the therapeutic agent can be a polypeptide encoded by a polynucleotide comprising polymorphic sequence selected from the group consisting of SEQ ID NOS:5, 8 and 11, or by a polynucleotide comprising a nucleotide sequence that is complementary to any one of polymorphic sequences SEQ ID NOS:5, 8 and 11, provided that the polymorphic sequence includes the nucleotide listed in Tables 5-7 for the polymorphic sequence.

The therapeutic agent may further include an antibody as herein described, or an oligonucleotide comprising a polymorphic sequence selected from the group consisting of SEQ ID NOS:5, 8 and 11, or by a polynucleotide comprising a nucleotide sequence that is complementary to any one of polymorphic sequences SEQ ID NOS:5, 8 and 11, provided that the polymorphic sequence includes the nucleotide listed in Tables 5-7 for the polymorphic sequence.

In another aspect, the invention provides an oligonucleotide array comprising one or more oligonucleotides hybridizing to a first polynucleotide at a polymorphic site encompassed therein. The first polynucleotide can be, e.g., a nucleotide sequence comprising one or more polymorphic sequences (SEQ ID NOS:5, 8 and 11); a nucleotide sequence that is a fragment of any of the nucleotide sequences, provided that the fragment includes a polymorphic site in the polymorphic sequence; a complementary nucleotide sequence comprising a sequence complementary to one or more polymorphic sequences (SEQ ID NOS:5, 8 and 11); or a nucleotide sequence that is a fragment of the complementary sequence, provided that the fragment includes a polymorphic site in the polymorphic sequence.

In preferred embodiments, the array comprises 10; 100; 1,000; 10,000; 100,000 or more oligonucleotides.

The invention also provides a kit comprising one or more of the herein-described nucleic acids. The kit can include, e.g., a polynucleotide which includes one or more of the SNPs described herein. The polynucleotide can be, e.g., a nucleotide sequence which includes one or more of the polymorphic sequences shown in Tables 5-7 (SEQ ID NOS: 5, 8 and 11) and which includes a polymorphic sequence, or a fragment of the polymorphic sequence, as long as it includes the polymorphic site. The polynucleotide may alternatively contain a nucleotide sequence which includes a sequence complementary to one or more of the sequences (SEQ ID NOS:5, 8 and 11), or a fragment of the complementary nucleotide sequence, provided that the fragment includes a polymorphic site in the polymorphic sequence. The invention provides an isolated allele-specific oligonucleotide that hybridizes to a first polynucleotide containing a polymorphic site. The first polynucleotide can be, e.g., a nucleotide sequence comprising one or more polymorphic sequences (SEQ ID NOS:5, 8 and 11), provided that the polymorphic sequence includes a nucleotide other than the nucleotide recited in Tables 5-7 for the polymorphic sequence. Alternatively, the first polynucleotide can be a nucleotide sequence that is a fragment of the polymorphic sequence, provided that the fragment includes a polymorphic site in the polymorphic sequence, or a complementary nucleotide sequence which includes a sequence complementary to one or more polymorphic sequences (SEQ ID NOS:5, 8 and 11) provided that the complementary nucleotide sequence includes a nucleotide other than the complement of the nucleotide recited in Tables 5-7. The first polynucleotide may in addition include a nucleotide sequence that is a fragment of the complementary sequence, provided that the fragment includes a polymorphic site in the polymorphic sequence.

In a further aspect, the invention includes a method for determining the presence of or predisposition to a disease or pathological condition associated with a polymorphism of SEQ ID NO:3, 6, or 9, the method comprising: (a) testing a biological sample from a mammalian subject for the presence of a polymorphism; and (b) determining the copy number of the polymorphic allele, wherein the copy number of the polymorphic allele indicates the presence of or predisposition to said disease or pathological condition. As used herein, copy number refers to the number of mutant alelles. That is, the number of alelles carrying the SNP mutation. For example, a subject could have two identical wild type alelles (homozygous), one wild type alelle and one mutant SNP alelle (heterozygous) or two mutant SNP alelles (homozygous). The invention also includes a method for identifying the carrier status of a genetic risk-altering factor associated with a polymorphism of SEQ ID NO:3, 6, or 9, the method comprising: (a) testing a biological sample from a mammalian subject for the presence of a polymorphism; and (b) determining the copy number of the polymorphic allele, wherein the copy number of the polymorphic allele indicates carrier status. In a preferred embodiment, the polymorphic alelle is indicative of increased serum levels of bicarbonate. In another embodiment, the disease or pathological condition is selected from the group consisting of respiratory and nonrespiratory alkalosis, respiratory and/or renal complications, cardiovascular disease, non-insulin dependent diabetes (Type II Diabetes), atherosclerosis, steatosis, hypertension, microvascular disease, and stroke.

In a further embodiment, the genetic risk factor is selected from the group consisting of increased serum levels of bicarbonate, a decrease in systolic blood pressure of 0.1 standard deviation below the mean level in the sampled population, a decrease in radial peripheral maximal dp/dt of 0.1 standard deviation below the mean level in the sampled population, and decreased BMI. In one aspect of the invention the polymorphic sequence is indicative of a decrease in systolic blood pressure or a decrease in radial peripheral maximal dp/dt of 0.1 standard deviation below the mean level in the sampled population. In another aspect, the polymorphic alelle is indicative of decreased BMI.

In another aspect, the invention provides a method of treating a subject suffering from, at risk for, or suspected of, suffering from a pathology ascribed to the presence of a sequence polymorphism in a subject, the method comprising: a) providing a subject suffering from a pathology associated with aberrant expression of a first nucleic acid comprising a polymorphic sequence selected from the group consisting of SEQ ID NOS:3, 5, 6, 8, 9, and 11, or its complement, and b) administering to the subject an effective therapeutic dose of a first nucleic acid comprising the polymorphic sequence, provided that the second nucleic acid comprises the nucleotide present in the wild type allele, thereby treating said subject.

The invention also includes a method of treating a subject suffering from, at risk for, or suspected of suffering from, a pathology ascribed to the presence of a sequence polymorphism in a subject, the method comprising: a) providing a subject suffering from, at risk for, or suspected of suffering from, a pathology associated with aberrant expression of a nucleic acid comprising a polymorphic sequence selected from the group consisting of SEQ ID NOS:3, 5, 6, 8, 9, and 11, or its complement, and b) administering to the subject an effective dose of an oligonucleotide comprising a polymorphic sequence selected from the group consisting of SEQ ID NOS:3, 5, 6, 8, 9, and 11, or by a polynucleotide comprising a nucleotide sequence that is complementary to any one of polymorphic sequences SEQ ID NOS:3, 5, 6, 8, 9, or 11, thereby treating said subject.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The present invention provides novel nucleotides and polypeptides encoded thereby. Included in the invention are a novel nucleic acid sequence and its encoded polypeptides. The sequences are collectively referred to herein as “NOV1 nucleic acids” or “NOV1 polynucleotides” and the corresponding encoded polypeptides are referred to as “NOV1 polypeptides” or “NOV1 proteins.” Unless indicated otherwise, “NOV1” is meant to refer to any of the novel sequences disclosed herein. Table 1 provides a summary of the NOV1 nucleic acids and their encoded polypeptides.

TABLE 1

NOV Polynucleotide and Polypeptide Sequences and
Corresponding SEQ ID Numbers

| | | | | |
|---|---|---|---|---|
| | | | SEQ ID | |
| | | SEQ ID | NO | |
| | | NO | (poly- | |
| Assign- | Internal | (nucleic | pep- | |
| ment | Identification | acid) | tide) | Homology |

| | | | | |
|---|---|---|---|---|
| 1 | CG105201-01 | 1 | 2 | Hexokinase 3 |

The invention also provides human SNPs in sequences which are transcribed, i.e., are cSNPs. Many SNPs have been identified in genes related to polypeptides of known function. If desired, SNPs associated with various polypeptides can be used together. For example, SNPs can be grouped according to whether they are derived from a nucleic acid encoding a polypeptide related to particular protein family or involved in a particular function. Similarly, SNPs can be grouped according to the functions played by their gene products. Such functions include, structural proteins, proteins from which associated with metabolic pathways fatty acid metabolism, glycolysis, intermediary metabolism, calcium metabolism, proteases, and amino acid metabolism, etc. Specifically, the present invention provides a number of human cSNPs based on at least one gene product that has not been previously identified. In contrast, and as defined specifically in the following paragraph, the cSNPs involve nucleic acid sequences that are assembled from at least one known sequence. Table 2 provides a summary of the SNPs of this invention.

TABLE 2

SNP Polynucleotide and Polypeptide Sequences and
Corresponding SEQ ID Numbers

| | | | | | |
|---|---|---|---|---|---|
| | | SEQ ID NO | | SEQ ID | |
| | | Reference | SEQ ID NO | NO: | |
| | | Polymorphic | Reference | Variant | |
| | | sequence | Polymorphic | SNP | |
| | Internal | (nucleic | sequence | (Nucleic | |
| Assignment | Identification | acid) | (polypeptide) | Acid) | Homology |

| | | | | | |
|---|---|---|---|---|---|
| 1 | 12252120 | 3 | 4 | 5 | Hexokinase 3 |
| 2 | 12252108 | 6 | 7 | 8 | SIAT1 (beta-galactosidase alpha- |
| | | | | 2,6-sialyltransferase) | |
| 3 | 12252123 | 9 | 10 | 11 | Peroxisomal Biogenesis Factor 6 |
| | | | | (PEX6, PEROXIN6) | |

Table 2 provides information concerning the allelic sequences. One of the sequences may be termed a reference polymorphic sequence, and the corresponding second sequence includes the variant SNP at the polymorphic site. The SEQ ID NOs. are also cross-referenced in Table 2. A reference to the SEQ ID NOs that corresponds to the translated amino acid sequence are also given. The Table includes descriptive information for each cSNP, each of which occupies one row in the Table.

The SNPs disclosed in this invention were detected by aligning large numbers of sequences from genetically diverse sources of publicly available mRNA libraries (Clontech). Software designed specifically to look for multiple examples of variant bases differing from a consensus sequence was created and deployed. A criteria of a minimum of two occurrences of a sequence differing from the consensus in high quality sequence reads was used to identify an SNP.

The SNPs described herein may be useful in diagnostic kits, for DNA arrays on chips and for other uses that involve hybridization of the SNP.

Specific SNPs may have utility where a disease has already been associated with that gene. Examples of possible disease correlations between the claimed SNPs with members of the genes of each classification are listed below:

A.) Hexokinase 3-Like Proteins

Hexokinase (ATP:D-hexose 6-phosphotransferase, EC 2.7.1.1) is an important enzyme that catalyses the ATP-dependent conversion of aldo- and keto-hexose sugars to the hexose-6-phosphate. The enzyme can catalyse this reaction on glucose, fructose, sorbitol and glucosamine, and as such is the first step in a number of metabolic pathways. The enzyme is widely distributed in eukaryotes. There are three isozymes of hexokinase in yeast (PI, PII and glucokinase): isozymes PI and PII phosphorylate both aldo- and keto-sugars; glucokinase is specific for aldo-hexoses. All three isozymes contain a single copy of the hexokinase domain. Structural studies of yeast hexokinase reveal a well-defined catalytic pocket that binds ATP and hexose, allowing easy transfer of the phosphate from ATP to the sugar.

In mammalian tissues hexokinase exists as four isoenzymes (designated I to IV) encoded by distinct genes. These proteins are homologous. Types I to III contain two repeats of the hexokinase domain, while hexokinase IV (sometimes incorrectly referred to as glucokinase) has only one. The N-terminus of types I to III is the regulatory- and the C-terminus is the catalytic-region. This organization is believed to be the result of a duplication and tandem fusion event involving the gene encoding for the ancestral hexokinase. Palma et al. (1996) cloned the carboxyl-domain of human hexokinase type III and expressed it in Escherichia coli as a glutathione S-transferase fusion protein, using the pGEX-2T expression vector. The recombinant protein showed catalytic activity. Palma et al. (1996) also performed a comparative study of the kinetic properties of the expressed carboxyl-domain and the enzyme partially purified from human lymphocytes. The results of Palma et al. (1996) allow a better understanding of the role of the carboxyl-domain in determining the catalytic properties of the enzyme. Complementary DNA clones encoding human hexokinase III were isolated by Furuta et al. (1996) from a liver cDNA library. There was 84.7% identity between the amino acid sequences of human and rat hexokinase III. RNA blotting showed the presence of hexokinase III mRNA in liver and lung. Fluorescence in situ hybridization localized the human hexokinase III gene (HK3) to chromosome 5, band q35.2.

NOV1 is a member of the hexokinase 3 family of genes. The nucleic acid sequence of NOV1 is a splice variant of gb:GENBANK-ID:HSU51333|acc:U51333.1 mRNA from Homo sapiens (Human hexokinase III (HK3) mRNA, complete cds) (See Example 1). This splice variation adds 143 extra bases (b) to the middle of the human hexokinase III mRNA (bases 1145-1287 of splice variant are not present in the public version). This variation results from alternative splicing in which an intron between two exons is not removed. Incorporation of this region introduces an early stop codon, thus truncating the protein from 923 amino acids to 357 amino acids as compared to ptnr:SWISSNEW-ACC:P52790 protein from Homo sapiens (Human) (HEXOKINASE TYPE III (EC 2.7.1.1) (HK III)).

The public hexokinase III contains two hexokinase domains (normal for the hexokinase III family), while the truncated version contains only 1 hexokinase domain. A disclosed NOV1 maps to chromosome 5. This assignment was made using mapping information associated with genomic clones, public genes and ESTs sharing sequence identity with the disclosed sequence and CuraGen Corporation's Electronic Northern bioinformatic tool.

A disclosed NOV1 is expressed in at least the following tissues: liver, lung, uterus, prostate, blood, metastatic melanoma to bowel, colon, spleen, lymph node. Expression information was derived from the tissue sources of the sequences that were included in the derivation of the sequence.

The protein similarity information, expression pattern, cellular localization, and map location for the protein and nucleic acid disclosed herein suggest that the disclosed NOV1 protein may have important structural and/or physiological functions characteristic of the Hexokinase III family. Therefore, the disclosed NOV1 nucleic acids and proteins are useful in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon.

NOV1 nucleic acids and proteins have applications in the diagnosis and/or treatment of various diseases and disorders. For example, the compositions of the present invention will have efficacy for the treatment of patients suffering from: metastatic melanoma, Von Hippel-Lindau (VHL) syndrome, cirrhosis, transplantation, systemic lupus erythematosus, autoimmune disease, asthma, emphysema, scleroderma, allergy, ARDS, endometriosis, fertility, anemia, ataxia-telangiectasia, autoimmume disease, immunodeficiencies, lymphedema, allergies, hemophilia, hypercoagulation, idiopathic thrombocytopenic purpura, immunodeficiencies, graft vesus host, cancer, trauma, regeneration (in vitro and in vivo), viral/bacterial/parasitic infections, as well as other diseases, disorders and conditions.

B.) SIAT1 (beta-galactosidase alpha-2,6-sialyltransferase)

The systolic blood pressure is significantly associated with this variant, with a statistical significance level of 0.0005. The radial peripheral maximal dp/dt is also significantly associated with this variant, with a statistical significance level of 0.002. The presence of this variant allele (SNP2, variant 12252108) is associated with a decrease in systolic blood pressure of 0.1 standard deviation below the mean level in the sampled population. Increased systolic blood pressure is a risk factor in cardiovascular disease, for example stroke or coronary heart disease, or other disorders that are secondarily affected by abnormal blood pressure. Therefore the SNP reported here may be a specific marker for a statistically significant decreased risk of cardiovascular disease.

C.) Peroxisomal Biogenesis Factor 6 (PEX6, PEROXIN6)

The invention also relates to an isolated nucleic acid molecule encoding Peroxisomal Biogenesis Factor 6 (PEX6, PEROXIN 6) having a nucleotide polymorphism (SNP3, variant 12252123) where the T allele is indicative of decreased Body Mass Index (BMI), and therefore a decreased risk for non-insulin dependent diabetes mellitus (Type II Diabetes), atherosclerosis, steatosis, hypertension, microvascular disease and stroke. The invention also relates to a method for identifying individuals who are carriers of the genetic risk-altering factor or are at decreased risk. The method includes obtaining a biological sample from an individual and testing the individual for the nucleotide polymorphism, wherein the disease risk may decrease with the dose of the T allele.

BMI, or Body Mass Index, is a measure of obesity and body fat. Obesity is a medical condition characterized by storage of excess body fat. The human body naturally stores fat tissue under the skin and around organs and joints. Fat is critical for good health because it is a source of energy when the body lacks the energy necessary to sustain life processes, and it provides insulation and protection for internal organs. However, the accumulation of too much fat in the body is associated with a wide variety of health problems and diseases. Studies show that individuals who are 20 percent or more overweight run a greater risk of developing diabetes mellitus, hypertension, coronary heart disease, stroke, arthritis, and some forms of cancer. According to the National Institutes of Health, in the United States 97 million adults are overweight or obese.

Most physicians use the body mass index (BMI) to determine desirable weights. BMI is calculated metrically as weight divided by [height]2, expressed in kilograms per meter-squared. People with a BMI of 25.0 to 29.9 are considered overweight and people with a BMI of 30 or above are considered obese. BMI is easily calculated, and many internet sites have interactive BMI calculators (e.g. National Heart, Lung, and Blood Institute http://www.nhlbisupport.com/bmi/).

The SNPs of the invention are shown in Example 2. The Tables in Example 2 provide a summary of the polymorphic sequences disclosed herein. In each Table, a “SNP” is a polymorphic site embedded in a polymorphic sequence. The polymorphic site is occupied by a single nucleotide, which is the position of nucleotide variation between the wild type and polymorphic allelic sequences. The site is usually preceded by and followed by relatively highly conserved sequences of the allele (e.g., sequences that vary in less than 1/100 or 1/1000 members of the populations). Thus, a polymorphic sequence can include one or more of the following sequences: (1) a sequence having the nucleotide denoted in the corresponding Table at the polymorphic site in the polymorphic sequence; or (2) a sequence having a nucleotide other than the nucleotide denoted in the Table at the polymorphic site in the polymorphic sequence. An example of the latter sequence is a polymorphic sequence having the nucleotide denoted in Table 4 at the polymorphic site in the polymorphic sequence.

Nucleotide sequences for a referenced-polymorphic pair are presented in Example 2. Each cSNP entry provides information concerning the wild type nucleotide sequence as well as the corresponding sequence that includes the SNP at the polymorphic site. The SEQ ID NOs: are also cross referenced in Table 2. A reference to the SEQ ID NOs: giving the translated amino acid sequences are also given if appropriate. The Tables include information that provide descriptive information for each cSNP, each of which occupies one row in the Table.

Provided herein are compositions which include, or are capable of detecting, nucleic acid sequences having these polymorphisms, as well as methods of using nucleic acids.

NOV1 Nucleic Acids and Polypeptides

One aspect of the invention pertains to isolated nucleic acid molecules that encode NOV1 polypeptides or biologically active portions thereof Also included in the invention are nucleic acid fragments sufficient for use as hybridization probes to identify NOV1-encoding nucleic acids (e.g., NOV1 mRNAs) and fragments for use as PCR primers for the amplification and/or mutation of NOV1 nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

A NOV1 nucleic acid can encode a mature NOV1 polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one, which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOV1 nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence SEQ ID NO: 1 or a complement of this aforementioned nucleotide sequence, can be isolated using standard molecular biology techniques and the sequence information provided herein. Using all or a portion of the nucleic acid sequence of SEQ ID NO: 1 as a hybridization probe, NOV1 molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to NOV1 nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides SEQ ID NO: 1, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NO: 1, or a portion of this nucleotide sequence (e.g., a fragment that can be used as a probe or primer or a fragment encoding a biologically-active portion of a NOV1 polypeptide). A nucleic acid molecule that is complementary to the nucleotide sequence shown NO: 1 is one that is sufficiently complementary to the nucleotide sequence shown NO: 1 that it can hydrogen bond with little or no mismatches to the nucleotide sequence shown SEQ ID NO: 1, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of NOV1 polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for a NOV1 polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding huma NOV1 protein. Homologous nucleic acid sequences include those nucleic acid sequences that encode conservative amino acid substitutions (see below) in SEQ ID NO: 1, as well as a polypeptide possessing NOV1 biological activity. Various biological activities of the NOV1 proteins are described below.

A NOV1 polypeptide is encoded by the open reading frame (“ORF”) of a NOV1 nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bonafide cellular protein, a minimum size requirement is often set, e.g., a stretch of DNA that would encode a protein of 50 amino acids or more.

The nucleotide sequences determined from the cloning of the huma NOV1 genes allows for the generation of probes and primers designed for use in identifying and/or cloning NOV1 homologues in other cell types, e.g. from other tissues, as well as NOV1 homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence SEQ ID NO: 1; or an anti-sense strand nucleotide sequence of SEQ ID NO: 1; or of a naturally occurring mutant of SEQ ID NO: 1.

Probes based on the huma NOV1 nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express a NOV1 protein, such as by measuring a level of a NOV1-encoding nucleic acid in a sample of cells from a subject e.g., detecting NOV1 mRNA levels or determining whether a genomic NOV1 gene has been mutated or deleted.

“A polypeptide having a biologically-active portion of a NOV1 polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOV1” can be prepared by isolating a portion SEQ ID NO:1, that encodes a polypeptide having a NOV1 biological activity (the biological activities of the NOV1 proteins are described below), expressing the encoded portion of NOV1 protein (e.g., by recombinant expression in vitro) and assessing the activity of the encoded portion of NOV1.

NOV1 Nucleic Acid and Polypeptide Variants

The invention further encompasses nucleic acid molecules that differ from the nucleotide sequences shown in SEQ ID NO: 1 due to degeneracy of the genetic code and thus encode the same NOV1 proteins as that encoded by the nucleotide sequences shown in SEQ ID NO:1. In another embodiment, an isolated nucleic acid molecule of the invention has a nucleotide sequence encoding a protein having an amino acid sequence shown in SEQ ID NO:2.

In addition to the huma NOV1 nucleotide sequences shown in SEQ ID NO: 1, it will be appreciated by those skilled in the art that DNA sequence polymorphisms that lead to changes in the amino acid sequences of the NOV1 polypeptides may exist within a population (e.g., the human population). Such genetic polymorphism in the NOV1 genes may exist among individuals within a population due to natural allelic variation. As used herein, the terms “gene” and “recombinant gene” refer to nucleic acid molecules comprising an open reading frame (ORF) encoding a NOV1 protein, preferably a vertebrate NOV1 protein. Such natural allelic variations can typically result in 1-5% variance in the nucleotide sequence of the NOV1 genes. Any and all such nucleotide variations and resulting amino acid polymorphisms in the NOV1 polypeptides, which are the result of natural allelic variation and that do not alter the functional activity of the NOV1 polypeptides, are intended to be within the scope of the invention.

Moreover, nucleic acid molecules encoding NOV1 proteins from other species, and thus that have a nucleotide sequence that differs from the human SEQ ID NO: 1 are intended to be within the scope of the invention. Nucleic acid molecules corresponding to natural allelic variants and homologues of the NOV1 cDNAs of the invention can be isolated based on their homology to the huma NOV1 nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NO: 1. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding NOV1 proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to the sequences SEQ ID NO: 1, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NO: 1, or fragments, analogs or derivatives thereof, under conditions of moderate stringency is provided. A non-limiting example of moderate stringency hybridization conditions are hybridization in 6×SSC, 5× Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Kriegler, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences SEQ ID NO: 1, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

Conservative Mutations

In addition to naturally-occurring allelic variants of NOV1 sequences that may exist in the population, the skilled artisan will further appreciate that changes can be introduced by mutation into the nucleotide sequences SEQ ID NO: 1, thereby leading to changes in the amino acid sequences of the encoded NOV1 proteins, without altering the functional ability of said NOV1 proteins. For example, nucleotide substitutions leading to amino acid substitutions at “non-essential” amino acid residues can be made in the sequence SEQ ID NO:2. A “non-essential” amino acid residue is a residue that can be altered from the wild-type sequences of the NOV1 proteins without altering their biological activity, whereas an “essential” amino acid residue is required for such biological activity. For example, amino acid residues that are conserved among the NOV1 proteins of the invention are predicted to be particularly non-amenable to alteration. Amino acids for which conservative substitutions can be made are well-known within the art.

Another aspect of the invention pertains to nucleic acid molecules encoding NOV1 proteins that contain changes in amino acid residues that are not essential for activity. Such NOV1 proteins differ in amino acid sequence from SEQ ID NO: 1 yet retain biological activity. In one embodiment, the isolated nucleic acid molecule comprises a nucleotide sequence encoding a protein, wherein the protein comprises an amino acid sequence at least about 45% homologous to the amino acid sequences SEQ ID NO:2. Preferably, the protein encoded by the nucleic acid molecule is at least about 60% homologous to SEQ ID NO:2; more preferably at least about 70% homologous SEQ ID NO:2; still more preferably at least about 80% homologous to SEQ ID NO:2; even more preferably at least about 90% homologous to SEQ ID NO:2; and most preferably at least about 95% homologous to SEQ ID NO:2.

An isolated nucleic acid molecule encoding a NOV1 protein homologous to the protein of SEQ ID NO:2 can be created by introducing one or more nucleotide substitutions, additions or deletions into the nucleotide sequence of SEQ ID NO: 1, such that one or more amino acid substitutions, additions or deletions are introduced into the encoded protein.

Mutations can be introduced into SEQ ID NO: 1 by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOV1 protein is replaced with another amino acid residue from the same side chain family. Alternatively, in another embodiment, mutations can be introduced randomly along all or part of a NOV1 coding sequence, such as by saturation mutagenesis, and the resultant mutants can be screened for NOV1 biological activity to identify mutants that retain activity. Following mutagenesis SEQ ID NO: 1, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, VLIM, HFY, wherein the letters within each group represent the single letter amino acid code. In one embodiment, a mutant NOV1 protein can be assayed for (i) the ability to form protein:protein interactions with other NOV1 proteins, other cell-surface proteins, or biologically-active portions thereof, (ii) complex formation between a mutant NOV1 protein and a NOV1 ligand; or (iii) the ability of a mutant NOV1 protein to bind to an intracellular target protein or biologically-active portion thereof; (e.g. avidin proteins).

In yet another embodiment, a mutant NOV1 protein can be assayed for the ability to regulate a specific biological function (e.g., regulation of insulin release).

Antisense Nucleic Acids

Another aspect of the invention pertains to isolated antisense nucleic acid molecules that are hybridizable to or complementary to the nucleic acid molecule comprising the nucleotide sequence of SEQ ID NO: 1, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOV1 coding strand, or to only a portion thereof. Nucleic acid molecules encoding fragments, homologs, derivatives and analogs of a NOV1 protein of SEQ ID NO:2, or antisense nucleic acids complementary to a NOV1 nucleic acid sequence of SEQ ID NO: 1, are additionally provided.

In one embodiment, an antisense nucleic acid molecule is antisense to a “coding region” of the coding strand of a nucleotide sequence encoding a NOV1 protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOV1 protein. The term “noncoding region” refers to 5′ and 3′ sequences which flank the coding region that are not translated into amino acids (i.e., also referred to as 5′ and 3′ untranslated regions).

Given the coding strand sequences encoding the NOV1 protein disclosed herein, antisense nucleic acids of the invention can be designed according to the rules of Watson and Crick or Hoogsteen base pairing. The antisense nucleic acid molecule can be complementary to the entire coding region of NOV1 mRNA, but more preferably is an oligonucleotide that is antisense to only a portion of the coding or noncoding region of NOV1 mRNA. For example, the antisense oligonucleotide can be complementary to the region surrounding the translation start site of NOV1 mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a NOV1 protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual P-units, the strands run parallel to each other. See, e.g., Gaultier, et al., 1987. Nucl. Acids Res. 15: 6625-6641. The antisense nucleic acid molecule can also comprise a 2′-o-methylribonucleotide (See, e.g., Inoue, et al. 1987. Nucl. Acids Res. 15: 6131-6148) or a chimeric RNA-DNA analogue (See, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOV1 mRNA transcripts to thereby inhibit translation of NOV1 mRNA. A ribozyme having specificity for a NOV1-encoding nucleic acid can be designed based upon the nucleotide sequence of a NOV1 cDNA disclosed herein (i.e., SEQ ID NO:1). For example, a derivative of a Tetrahymena L-19 IVS RNA can be constructed in which the nucleotide sequence of the active site is complementary to the nucleotide sequence to be cleaved in a NOV1-encoding mRNA. See, e.g., U.S. Pat. Nos. 4,987,071 to Cech, et al. and 5,116,742 to Cech, et al. NOV1 mRNA can also be used to select a catalytic RNA having a specific ribonuclease activity from a pool of RNA molecules. See, e.g., Bartel et al., (1993) Science 261:1411-1418.

Alternatively, NOV1 gene expression can be inhibited by targeting nucleotide sequences complementary to the regulatory region of the NOV1 nucleic acid (e.g., the NOV1 promoter and/or enhancers) to form triple helical structures that prevent transcription of the NOV1 gene in target cells. See, e.g., Helene, 1991. Anticancer Drug Des. 6: 569-84; Helene, et al. 1992. Ann. N.Y. Acad. Sci. 660: 27-36; Maher, 1992. Bioassays 14: 807-15.

In various embodiments, the NOV1 nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675. PNAs of NOV1 can be used in therapeutic and diagnostic applications. For example, PNAs can be used as antisense or antigene agents for sequence-specific modulation of gene expression by, e.g., inducing transcription or translation arrest or inhibiting replication. PNAs of NOV1 can also be used, for example, in the analysis of single base pair mutations in a gene (e.g., PNA directed PCR clamping; as artificial restriction enzymes when used in combination with other enzymes, e.g., S1 nucleases (See, Hyrup, et al., 1996.supra); or as probes or primers for DNA sequence and hybridization (See, Hyrup, et al., 1996, supra; Perry-O'Keefe, et al., 1996. supra).

In another embodiment, PNAs of NOV1 can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOV1 can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOV1 Polypeptides

A polypeptide according to the invention includes a polypeptide including the amino acid sequence of NOV1 polypeptides whose sequences are provided in SEQ ID NO:2. The invention also includes a mutant or variant protein any of whose residues may be changed from the corresponding residues shown in SEQ ID NO:2 while still encoding a protein that maintains its NOV1 activities and physiological functions, or a functional fragment thereof.

In general, a NOV1 variant that preserves NOV1-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOV1 proteins, and biologically-active portions thereof, or derivatives, fragments, analogs or homologs thereof. Also provided are polypeptide fragments suitable for use as immunogens to raise anti-NOV1 antibodies. In one embodiment, native NOV1 proteins can be isolated from cells or tissue sources by an appropriate purification scheme using standard protein purification techniques. In another embodiment, NOV1 proteins are produced by recombinant DNA techniques. Alternative to recombinant expression, a NOV1 protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOV1 protein is derived, or substantially free from chemical precursors or other chemicals when chemically synthesized. The language “substantially free of cellular material” includes preparations of NOV1 proteins in which the protein is separated from cellular components of the cells from which it is isolated or recombinantly-produced. In one embodiment, the language “substantially free of cellular material” includes preparations of NOV1 proteins having less than about 30% (by dry weight) of non-NOV1 proteins (also referred to herein as a “contaminating protein”), more preferably less than about 20% of non-NOV1 proteins, still more preferably less than about 10% of non-NOV1 proteins, and most preferably less than about 5% of non-NOV1 proteins. When the NOV1 protein or biologically-active portion thereof is recombinantly-produced, it is also preferably substantially free of culture medium, i.e., culture medium represents less than about 20%, more preferably less than about 10%, and most preferably less than about 5% of the volume of the NOV1 protein preparation.

The language “substantially free of chemical precursors or other chemicals” includes preparations of NOV1 proteins in which the protein is separated from chemical precursors or other chemicals that are involved in the synthesis of the protein. In one embodiment, the language “substantially free of chemical precursors or other chemicals” includes preparations of NOV1 proteins having less than about 30% (by dry weight) of chemical precursors or non-NOV1 chemicals, more preferably less than about 20% chemical precursors or non-NOV1 chemicals, still more preferably less than about 10% chemical precursors or non-NOV1 chemicals, and most preferably less than about 5% chemical precursors or non-NOV1 chemicals.

Biologically-active portions of NOV1 proteins include peptides comprising amino acid sequences sufficiently homologous to or derived from the amino acid sequences of the NOV1 proteins (e.g., the amino acid sequence shown in SEQ ID NO:2) that include fewer amino acids than the full-length NOV1 proteins, and exhibit at least one activity of a NOV1 protein. Typically, biologically-active portions comprise a domain or motif with at least one activity of the NOV1 protein. A biologically-active portion of a NOV1 protein can be a polypeptide which is, for example, 10, 25, 50, 100 or more amino acid residues in length. Moreover, other biologically-active portions, in which other regions of the protein are deleted, can be prepared by recombinant techniques and evaluated for one or more of the functional activities of a native NOV1 protein.

In an embodiment, the NOV1 protein has an amino acid sequence shown SEQ ID NO:2. In other embodiments, the NOV1 protein is substantially homologous to SEQ ID NO:2, and retains the functional activity of the protein of SEQ ID NO:2, yet differs in amino acid sequence due to natural allelic variation or mutagenesis, as described in detail, below. Accordingly, in another embodiment, the NOV1 protein is a protein that comprises an amino acid sequence at least about 45% homologous to the amino acid sequence SEQ ID NO:2, and retains the functional activity of the NOV1 proteins of SEQ ID NO:2.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NO: 1.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOV1 chimeric or fusion proteins. As used herein, a NOV1 “chimeric protein” or “fusion protein” comprises a NOV1 polypeptide operatively-linked to a non-NOV1 polypeptide. An “NOV1 polypeptide” refers to a polypeptide having an amino acid sequence corresponding to a NOV1 protein SEQ ID NO:2, whereas a “non-NOV1 polypeptide” refers to a polypeptide having an amino acid sequence corresponding to a protein that is not substantially homologous to the NOV1 protein, e.g., a protein that is different from the NOV1 protein and that is derived from the same or a different organism. Within a NOV1 fusion protein the NOV1 polypeptide can correspond to all or a portion of a NOV1 protein. In one embodiment, a NOV1 fusion protein comprises at least one biologically-active portion of a NOV1 protein. In another embodiment, a NOV1 fusion protein comprises at least two biologically-active portions of a NOV1 protein. In yet another embodiment, a NOV1 fusion protein comprises at least three biologically-active portions of a NOV1 protein. Within the fusion protein, the term “operatively-linked” is intended to indicate that the NOV1 polypeptide and the non-NOV1 polypeptide are fused in-frame with one another. The non-NOV1 polypeptide can be fused to the N-terminus or C-terminus of the NOV1 polypeptide.

In one embodiment, the fusion protein is a GST-NOV1 fusion protein in which the NOV1 sequences are fused to the C-terminus of the GST (glutathione S-transferase) sequences. Such fusion proteins can facilitate the purification of recombinant NOV1 polypeptides.

In another embodiment, the fusion protein is a NOV1 protein containing a heterologous signal sequence at its N-terminus. In certain host cells (e.g., mammalian host cells), expression and/or secretion of NOV1 can be increased through use of a heterologous signal sequence.

In yet another embodiment, the fusion protein is a NOV1-immunoglobulin fusion protein in which the NOV1 sequences are fused to sequences derived from a member of the immunoglobulin protein family. The NOV1-immunoglobulin fusion proteins of the invention can be incorporated into pharmaceutical compositions and administered to a subject to inhibit an interaction between a NOV1 ligand and a NOV1 protein on the surface of a cell, to thereby suppress NOV1-mediated signal transduction in vivo. The NOV1-immunoglobulin fusion proteins can be used to affect the bioavailability of a NOV1 cognate ligand. Inhibition of the NOV1 ligand/NOV1 interaction may be useful therapeutically for both the treatment of proliferative and differentiative disorders, as well as modulating (e.g. promoting or inhibiting) cell survival. Moreover, the NOV1-immunoglobulin fusion proteins of the invention can be used as immunogens to produce anti-NOV1 antibodies in a subject, to purify NOV1 ligands, and in screening assays to identify molecules that inhibit the interaction of NOV1 with a NOV1 ligand.

A NOV1 chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A NOV1-encoding nucleic acid can be cloned into such an expression vector such that the fusion moiety is linked in-frame to the NOV1 protein.

NOV1 Agonists and Antagonists

The invention also pertains to variants of the NOV1 proteins that function as either NOV1 agonists (i.e., mimetics) or as NOV1 antagonists. Variants of the NOV1 protein can be generated by mutagenesis (e.g., discrete point mutation or truncation of the NOV1 protein). An agonist of the NOV1 protein can retain substantially the same, or a subset of, the biological activities of the naturally occurring form of the NOV1 protein. An antagonist of the NOV1 protein can inhibit one or more of the activities of the naturally occurring form of the NOV1 protein by, for example, competitively binding to a downstream or upstream member of a cellular signaling cascade which includes the NOV1 protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOV1 proteins.

Variants of the NOV1 proteins that function as either NOV1 agonists (i.e., mimetics) or as NOV1 antagonists can be identified by screening combinatorial libraries of mutants (e.g., truncation mutants) of the NOV1 proteins for NOV1 protein agonist or antagonist activity. In one embodiment, a variegated library of NOV1 variants is generated by combinatorial mutagenesis at the nucleic acid level and is encoded by a variegated gene library. A variegated library of NOV1 variants can be produced by, for example, enzymatically ligating a mixture of synthetic oligonucleotides into gene sequences such that a degenerate set of potential NOV1 sequences is expressible as individual polypeptides, or alternatively, as a set of larger fusion proteins (e.g., for phage display) containing the set of NOV1 sequences therein. There are a variety of methods which can be used to produce libraries of potential NOV1 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOV1 sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the NOV1 protein coding sequences can be used to generate a variegated population of NOV1 fragments for screening and subsequent selection of variants of a NOV1 protein. In one embodiment, a library of coding sequence fragments can be generated by treating a double stranded PCR fragment of a NOV1 coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S1 nuclease, and ligating the resulting fragment library into an expression vector. By this method, expression libraries can be derived which encodes N-terminal and internal fragments of various sizes of the NOV1 proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOV1 proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOV1 variants. See, e.g., Arkin and Yourvan, 1992. Proc. Natl. Acad. Sci. USA 89: 7811-7815; Delgrave, et al., 1993. Protein Engineering 6:327-331.

Anti-NOV1 Antibodies

Also included in the invention are antibodies to NOV1 proteins, or fragments of NOV1 proteins. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab}′ and F_{(ab′)2} fragments, and an F_{ab} expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated NOV1-related protein of the invention may be intended to serve as an antigen, or a portion or fragment thereof, and additionally can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOV1-related protein that is located on the surface of the protein, e.g., a hydrophilic region. A hydrophobicity analysis of the huma NOV1-related protein sequence will indicate which regions of a NOV1-related protein are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each of which is incorporated herein by reference in its entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow and Lane, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, MONOCLONAL ANTIBODIES: PRINCIPLES AND PRACTICE, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., MONOCLONAL ANTIBODY PRODUCTION TECHNIQUES AND APPLICATIONS, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Preferably, antibodies having a high degree of specificity and a high binding affinity for the target antigen are isolated.

After the desired hybridoma cells are identified, the clones can be subcloned by limiting dilution procedures and grown by standard methods. Suitable culture media for this purpose include, for example, Dulbecco's Modified Eagle's Medium and RPMI-1640 medium. Alternatively, the hybridoma cells can be grown in vivo as ascites in a mammal. The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies relate to antibody molecules in which essentially the entire sequences of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al,(Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker. A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., 1991 EMBO J., 10:3655-3659.

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of a NOV1 protein is facilitated by generation of hybridomas that bind to the fragment of a NOV1 protein possessing such a domain. Thus, antibodies that are specific for a desired domain within a NOV1 protein, or derivatives, fragments, analogs or homologs thereof, are also provided herein.

Anti-NOV1 antibodies may be used in methods known within the art relating to the localization and/or quantitation of a NOV1 protein (e.g., for use in measuring levels of the NOV1 protein within appropriate physiological samples, for use in diagnostic methods, for use in imaging the protein, and the like). In a given embodiment, antibodies for NOV1 proteins, or derivatives, fragments, analogs or homologs thereof, that contain the antibody derived binding domain, are utilized as pharmacologically-active compounds (hereinafter “Therapeutics”).

An anti-NOV1 antibody (e.g., monoclonal antibody) can be used to isolate a NOV1 polypeptide by standard techniques, such as affinity chromatography or immunoprecipitation. An anti-NOV1 antibody can facilitate the purification of natural NOV1 polypeptide from cells and of recombinantly-produced NOV1 polypeptide expressed in host cells. Moreover, an anti-NOV1 antibody can be used to detect NOV1 protein (e.g., in a cellular lysate or cell supernatant) in order to evaluate the abundance and pattern of expression of the NOV1 protein. Anti-NOV1 antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, α-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

NOV1 Recombinant Expression Vectors and Host Cells

Another aspect of the invention pertains to vectors, preferably expression vectors, containing a nucleic acid encoding a NOV1 protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOV1 proteins, mutant forms of NOV1 proteins, fusion proteins, etc.).

The recombinant expression vectors of the invention can be designed for expression of NOV1 proteins in prokaryotic or eukaryotic cells. For example, NOV1 proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase. Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein. Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOV1 expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOV1 can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Carnpes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOV1 mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOV1 protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOV1 or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOV1 protein. Accordingly, the invention further provides methods for producing NOV1 protein using the host cells of the invention. In one embodiment, the method comprises culturing the host cell of invention (into which a recombinant expression vector encoding NOV1 protein has been introduced) in a suitable medium such that NOV1 protein is produced. In another embodiment, the method further comprises isolating NOV1 protein from the medium or the host cell.

Transgenic NOV1 Animals

The host cells of the invention can also be used to produce non-human transgenic animals. For example, in one embodiment, a host cell of the invention is a fertilized oocyte or an embryonic stem cell into which NOV1 protein-coding sequences have been introduced. Such host cells can then be used to create non-human transgenic animals in which exogenous NOV1 sequences have been introduced into their genome or homologous recombinant animals in which endogenous NOV1 sequences have been altered. Such animals are useful for studying the function and/or activity of NOV1 protein and for identifying and/or evaluating modulators of NOV1 protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOV1 gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOV1-encoding nucleic acid into the male pronuclei of a fertilized oocyte (e.g., by microinjection, retroviral infection) and allowing the oocyte to develop in a pseudopregnant female foster animal. The huma NOV1 cDNA sequences SEQ ID NO: 1 can be introduced as a transgene into the genome of a non-human animal. Alternatively, a non-human homologue of the huma NOV1 gene, such as a mouse NOV1 gene, can be isolated based on hybridization to the huma NOV1 cDNA (described further supra) and used as a transgene. Intronic sequences and polyadenylation signals can also be included in the transgene to increase the efficiency of expression of the transgene. A tissue-specific regulatory sequence(s) can be operably-linked to the NOV1 transgene to direct expression of NOV1 protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOV1 transgene in its genome and/or expression of NOV1 mRNA in tissues or cells of the animals. A transgenic founder animal can then be used to breed additional animals carrying the transgene. Moreover, transgenic animals carrying a transgene-encoding NOV1 protein can further be bred to other transgenic animals carrying other transgenes.

To create a homologous recombinant animal, a vector is prepared which contains at least a portion of a NOV1 gene into which a deletion, addition or substitution has been introduced to thereby alter, e.g., functionally disrupt, the NOV1 gene. The NOV1 gene can be a human gene (e.g., the cDNA of SEQ ID NO: 1), but more preferably, is a non-human homologue of a huma NOV1 gene. For example, a mouse homologue of huma NOV1 gene of SEQ ID NO: 1 can be used to construct a homologous recombination vector suitable for altering an endogenous NOV1 gene in the mouse genome. In one embodiment, the vector is designed such that, upon homologous recombination, the endogenous NOV1 gene is functionally disrupted (i.e., no longer encodes a functional protein; also referred to as a “knock out” vector).

Alternatively, the vector can be designed such that, upon homologous recombination, the endogenous NOV1 gene is mutated or otherwise altered but still encodes functional protein (e.g., the upstream regulatory region can be altered to thereby alter the expression of the endogenous NOV1 protein). In the homologous recombination vector, the altered portion of the NOV1 gene is flanked at its 5′- and 3′-termini by additional nucleic acid of the NOV1 gene to allow for homologous recombination to occur between the exogenous NOV1 gene carried by the vector and an endogenous NOV1 gene in an embryonic stem cell. The additional flanking NOV1 nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOV1 gene has homologously-recombined with the endogenous NOV1 gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOV1 nucleic acid molecules, NOV1 proteins, and anti-NOV1 antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a NOV1 protein or anti-NOV1 antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOV1 protein (e.g., via a recombinant expression vector in a host cell in gene therapy applications), to detect NOV1 mRNA (e.g., in a biological sample) or a genetic lesion in a NOV1 gene, and to modulate NOV1 activity, as described further, below. In addition, the NOV1 proteins can be used to screen drugs or compounds that modulate the NOV1 protein activity or expression as well as to treat disorders characterized by insufficient or excessive production of NOV1 protein or production of NOV1 protein forms that have decreased or aberrant activity compared to NOV1 wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease (possesses anti-microbial activity) and the various dyslipidemias. In addition, the anti-NOV1 antibodies of the invention can be used to detect and isolate NOV1 proteins and modulate NOV1 activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOV1 proteins or have a stimulatory or inhibitory effect on, e.g., NOV1 protein expression or NOV1 protein activity. The invention also includes compounds identified in the screening assays described herein. In one embodiment, the invention provides assays for screening candidate or test compounds which bind to or modulate the activity of the membrane-bound form of a NOV1 protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37:1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOV1 protein, or a biologically-active portion thereof, on the cell surface is contacted with a test compound and the ability of the test compound to bind to a NOV1 protein determined. The cell, for example, can of mammalian origin or a yeast cell. Determining the ability of the test compound to bind to the NOV1 protein can be accomplished, for example, by coupling the test compound with a radioisotope or enzymatic label such that binding of the test compound to the NOV1 protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOV1 protein, or a biologically-active portion thereof, on the cell surface with a known compound which binds NOV1 to form an assay mixture, contacting the assay mixture with a test compound, and determining the ability of the test compound to interact with a NOV1 protein, wherein determining the ability of the test compound to interact with a NOV1 protein comprises determining the ability of the test compound to preferentially bind to NOV1 protein or a biologically-active portion thereof as compared to the known compound.

In another embodiment, an assay is a cell-based assay comprising contacting a cell expressing a membrane-bound form of NOV1 protein, or a biologically-active portion thereof, on the cell surface with a test compound and determining the ability of the test compound to modulate (e.g., stimulate or inhibit) the activity of the NOV1 protein or biologically-active portion thereof. Determining the ability of the test compound to modulate the activity of NOV1 or a biologically-active portion thereof can be accomplished, for example, by determining the ability of the NOV1 protein to bind to or interact with a NOV1 target molecule. As used herein, a “target molecule” is a molecule with which a NOV1 protein binds or interacts in nature, for example, a molecule on the surface of a cell which expresses a NOV1 interacting protein, a molecule on the surface of a second cell, a molecule in the extracellular milieu, a molecule associated with the internal surface of a cell membrane or a cytoplasmic molecule. A NOV1 target molecule can be a non-NOV1 molecule or a NOV1 protein or polypeptide of the invention. In one embodiment, a NOV1 target molecule is a component of a signal transduction pathway that facilitates transduction of an extracellular signal (e.g. a signal generated by binding of a compound to a membrane-bound NOV1 molecule) through the cell membrane and into the cell. The target, for example, can be a second intercellular protein that has catalytic activity or a protein that facilitates the association of downstream signaling molecules with NOV1.

Determining the ability of the NOV1 protein to bind to or interact with a NOV1 target molecule can be accomplished by one of the methods described above for determining direct binding. In one embodiment, determining the ability of the NOV1 protein to bind to or interact with a NOV1 target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising a NOV1-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting a NOV1 protein or biologically-active portion thereof with a test compound and determining the ability of the test compound to bind to the NOV1 protein or biologically-active portion thereof. Binding of the test compound to the NOV1 protein can be determined either directly or indirectly as described above. In one such embodiment, the assay comprises contacting the NOV1 protein or biologically-active portion thereof with a known compound which binds NOV1 to form an assay mixture, contacting the assay mixture with a test compound, and determining the ability of the test compound to interact with a NOV1 protein, wherein determining the ability of the test compound to interact with a NOV1 protein comprises determining the ability of the test compound to preferentially bind to NOV1 or biologically-active portion thereof as compared to the known compound.

In still another embodiment, an assay is a cell-free assay comprising contacting NOV1 protein or biologically-active portion thereof with a test compound and determining the ability of the test compound to modulate (e.g. stimulate or inhibit) the activity of the NOV1 protein or biologically-active portion thereof. Determining the ability of the test compound to modulate the activity of NOV1 can be accomplished, for example, by determining the ability of the NOV1 protein to bind to a NOV1 target molecule by one of the methods described above for determining direct binding. In an alternative embodiment, determining the ability of the test compound to modulate the activity of NOV1 protein can be accomplished by determining the ability of the NOV1 protein further modulate a NOV1 target molecule. For example, the catalytic/enzymatic activity of the target molecule on an appropriate substrate can be determined as described, supra.

In yet another embodiment, the cell-free assay comprises contacting the NOV1 protein or biologically-active portion thereof with a known compound which binds NOV1 protein to form an assay mixture, contacting the assay mixture with a test compound, and determining the ability of the test compound to interact with a NOV1 protein, wherein determining the ability of the test compound to interact with a NOV1 protein comprises determining the ability of the NOV1 protein to preferentially bind to or modulate the activity of a NOV1 target molecule.

The cell-free assays of the invention are amenable to use of both the soluble form or the membrane-bound form of NOV1 protein. In the case of cell-free assays comprising the membrane-bound form of NOV1 protein, it may be desirable to utilize a solubilizing agent such that the membrane-bound form of NOV1 protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl—N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOV1 protein or its target molecule to facilitate separation of complexed from uncomplexed forms of one or both of the proteins, as well as to accommodate automation of the assay. Binding of a test compound to NOV1 protein, or interaction of NOV1 protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOV1 fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOV1 protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOV1 protein binding or activity determined using standard techniques.

Other techniques for immobilizing proteins on matrices can also be used in the screening assays of the invention. For example, either the NOV1 protein or its target molecule can be immobilized utilizing conjugation of biotin and streptavidin. Biotinylated NOV1 protein or target molecules can be prepared from biotin-NHS(N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOV1 protein or target molecules, but which do not interfere with binding of the NOV1 protein to its target molecule, can be derivatized to the wells of the plate, and unbound target or NOV1 protein trapped in the wells by antibody conjugation. Methods for detecting such complexes, in addition to those described above for the GST-immobilized complexes, include immunodetection of complexes using antibodies reactive with the NOV1 protein or target molecule, as well as enzyme-linked assays that rely on detecting an enzymatic activity associated with the NOV1 protein or target molecule.

In another embodiment, modulators of NOV1 protein expression are identified in a method wherein a cell is contacted with a candidate compound and the expression of NOV1 mRNA or protein in the cell is determined. The level of expression of NOV1 mRNA or protein in the presence of the candidate compound is compared to the level of expression of NOV1 mRNA or protein in the absence of the candidate compound. The candidate compound can then be identified as a modulator of NOV1 mRNA or protein expression based upon this comparison. For example, when expression of NOV1 mRNA or protein is greater (i.e., statistically significantly greater) in the presence of the candidate compound than in its absence, the candidate compound is identified as a stimulator of NOV1 mRNA or protein expression. Alternatively, when expression of NOV1 mRNA or protein is less (statistically significantly less) in the presence of the candidate compound than in its absence, the candidate compound is identified as an inhibitor of NOV1 mRNA or protein expression. The level of NOV1 mRNA or protein expression in the cells can be determined by methods described herein for detecting NOV1 mRNA or protein.

In yet another aspect of the invention, the NOV1 proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOV1 (“NOV1-binding proteins” or “NOV1-bp”) and modulate NOV1 activity. Such NOV1-binding proteins are also likely to be involved in the propagation of signals by the NOV1 proteins as, for example, upstream or downstream elements of the NOV1 pathway.

The two-hybrid system is based on the modular nature of most transcription factors, which consist of separable DNA-binding and activation domains. Briefly, the assay utilizes two different DNA constructs. In one construct, the gene that codes for NOV1 is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a NOV1-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOV1.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOV1 sequences, SEQ ID NO: 1, or fragments or derivatives thereof, can be used to map the location of the NOV1 genes, respectively, on a chromosome. The mapping of the NOV1 sequences to chromosomes is an important first step in correlating these sequences with genes associated with disease.

Briefly, NOV1 genes can be mapped to chromosomes by preparing PCR primers (preferably 15-25 bp in length) from the NOV1 sequences. Computer analysis of the NOV1, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOV1 sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOV1 sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOV1 gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOV1 sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057). Furthermore, the sequences of the invention can be used to provide an alternative technique that determines the actual base-by-base DNA sequence of selected portions of an individual's genome. Thus, the NOV1 sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOV1 sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NO: 1 are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOV1 protein and/or nucleic acid expression as well as NOV1 activity, in the context of a biological sample (e.g., blood, serum, cells, tissue) to thereby determine whether an individual is afflicted with a disease or disorder, or is at risk of developing a disorder, associated with aberrant NOV1 expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOV1 protein, nucleic acid expression or activity. For example, mutations in a NOV1 gene can be assayed in a biological sample. Such assays can be used for prognostic or predictive purpose to thereby prophylactically treat an individual prior to the onset of a disorder characterized by or associated with NOV1 protein, nucleic acid expression, or biological activity.

Another aspect of the invention provides methods for determining NOV1 protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of NOV1 in clinical trials. These and other agents are described in further detail in the following sections.

Diagnostic Assays

An exemplary method for detecting the presence or absence of NOV1 in a biological sample involves obtaining a biological sample from a test subject and contacting the biological sample with a compound or an agent capable of detecting NOV1 protein or nucleic acid (e.g., mRNA, genomic DNA) that encodes NOV1 protein such that the presence of NOV1 is detected in the biological sample. An agent for detecting NOV1 mRNA or genomic DNA is a labeled nucleic acid probe capable of hybridizing to NOV1 mRNA or genomic DNA. The nucleic acid probe can be, for example, a full-length NOV1 nucleic acid, such as the nucleic acid of SEQ ID NO:1, or a portion thereof, such as an oligonucleotide of at least 15, 30, 50, 100, 250 or 500 nucleotides in length and sufficient to specifically hybridize under stringent conditions to NOV1 mRNA or genomic DNA. Other suitable probes for use in the diagnostic assays of the invention are described herein.

An agent for detecting NOV1 protein is an antibody capable of binding to NOV1 protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOV1 mRNA, protein, or genomic DNA in a biological sample in vitro as well as in vivo. For example, in vitro techniques for detection of NOV1 mRNA include Northern hybridizations and in situ hybridizations. In vitro techniques for detection of NOV1 protein include enzyme linked immunosorbent assays (ELISAs), Western blots, immunoprecipitations, and immunofluorescence. In vitro techniques for detection of NOV1 genomic DNA include Southern hybridizations. Furthermore, in vivo techniques for detection of NOV1 protein include introducing into a subject a labeled anti-NOV1 antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject. In another embodiment, the methods further involve obtaining a control biological sample from a control subject, contacting the control sample with a compound or agent capable of detecting NOV1 protein, mRNA, or genomic DNA, such that the presence of NOV1 protein, mRNA or genomic DNA is detected in the biological sample, and comparing the presence of NOV1 protein, mRNA or genomic DNA in the control sample with the presence of NOV1 protein, mRNA or genomic DNA in the test sample.

The invention also encompasses kits for detecting the presence of NOV1 in a biological sample. For example, the kit can comprise: a labeled compound or agent capable of detecting NOV1 protein or mRNA in a biological sample; means for determining the amount of NOV1 in the sample; and means for comparing the amount of NOV1 in the sample with a standard. The compound or agent can be packaged in a suitable container. The kit can further comprise instructions for using the kit to detect NOV1 protein or nucleic acid.

Prognostic Assays

The diagnostic methods described herein can furthermore be utilized to identify subjects having or at risk of developing a disease or disorder associated with aberrant NOV1 expression or activity. For example, the assays described herein, such as the preceding diagnostic assays or the following assays, can be utilized to identify a subject having or at risk of developing a disorder associated with NOV1 protein, nucleic acid expression or activity. Alternatively, the prognostic assays can be utilized to identify a subject having or at risk for developing a disease or disorder. Thus, the invention provides a method for identifying a disease or disorder associated with aberrant NOV1 expression or activity in which a test sample is obtained from a subject and NOV1 protein or nucleic acid (e.g., mRNA, genomic DNA) is detected, wherein the presence of NOV1 protein or nucleic acid is diagnostic for a subject having or at risk of developing a disease or disorder associated with aberrant NOV1 expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOV1 expression or activity. For example, such methods can be used to determine whether a subject can be effectively treated with an agent for a disorder. Thus, the invention provides methods for determining whether a subject can be effectively treated with an agent for a disorder associated with aberrant NOV1 expression or activity in which a test sample is obtained and NOV1 protein or nucleic acid is detected (e.g., wherein the presence of NOV1 protein or nucleic acid is diagnostic for a subject that can be administered the agent to treat a disorder associated with aberrant NOV1 expression or activity).

The methods of the invention can also be used to detect genetic lesions in a NOV1 gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding a NOV1-protein, or the misexpression of the NOV1 gene. For example, such genetic lesions can be detected by ascertaining the existence of at least one of: (i) a deletion of one or more nucleotides from a NOV1 gene; (ii) an addition of one or more nucleotides to a NOV1 gene; (iii) a substitution of one or more nucleotides of a NOV1 gene, (iv) a chromosomal rearrangement of a NOV1 gene; (v) an alteration in the level of a messenger RNA transcript of a NOV1 gene, (vi) aberrant modification of a NOV1 gene, such as of the methylation pattern of the genomic DNA, (vii) the presence of a non-wild-type splicing pattern of a messenger RNA transcript of a NOV1 gene, (viii) a non-wild-type level of a NOV1 protein, (ix) allelic loss of a NOV1 gene, and (x) inappropriate post-translational modification of a NOV1 protein. As described herein, there are a large number of assay techniques known in the art which can be used for detecting lesions in a NOV1 gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOV1-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to a NOV1 gene under conditions such that hybridization and amplification of the NOV1 gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a NOV1 gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOV1 can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in NOV1 can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOV1 gene and detect mutations by comparing the sequence of the sample NOV1 with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOV1 gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOV1 sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOV1 cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on a NOV1 sequence, e.g., a wild-type NOV1 sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOV1 genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOV1 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a NOV1 gene. Furthermore, any cell type or tissue, preferably peripheral blood leukocytes, in which NOV1 is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOV1 activity (e.g., NOV1 gene expression), as identified by a screening assay described herein can be administered to individuals to treat (prophylactically or therapeutically) disorders (The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers.) In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOV1 protein, expression of NOV1 nucleic acid, or mutation content of NOV1 genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C 19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOV1 protein, expression of NOV1 nucleic acid, or mutation content of NOV1 genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a NOV1 modulator, such as a modulator identified by one of the exemplary screening assays described herein.

Monitoring of Effects During Clinical Trials

Monitoring the influence of agents (e.g., drugs, compounds) on the expression or activity of NOV1 (e.g., the ability to modulate aberrant cell proliferation and/or differentiation) can be applied not only in basic drug screening, but also in clinical trials. For example, the effectiveness of an agent determined by a screening assay as described herein to increase NOV1 gene expression, protein levels, or upregulate NOV1 activity, can be monitored in clinical trails of subjects exhibiting decreased NOV1 gene expression, protein levels, or downregulated NOV1 activity. Alternatively, the effectiveness of an agent determined by a screening assay to decrease NOV1 gene expression, protein levels, or downregulate NOV1 activity, can be monitored in clinical trails of subjects exhibiting increased NOV1 gene expression, protein levels, or upregulated NOV1 activity. In such clinical trials, the expression or activity of NOV1 and, preferably, other genes that have been implicated in, for example, a cellular proliferation or immune disorder can be used as a “read out” or markers of the immune responsiveness of a particular cell.

By way of example, and not of limitation, genes, including NOV1, that are modulated in cells by treatment with an agent (e.g., compound, drug or small molecule) that modulates NOV1 activity (e.g., identified in a screening assay as described herein) can be identified. Thus, to study the effect of agents on cellular proliferation disorders, for example, in a clinical trial, cells can be isolated and RNA prepared and analyzed for the levels of expression of NOV1 and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOV1 or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of a NOV1 protein, mRNA, or genomic DNA in the preadministration sample; (iii) obtaining one or more post-administration samples from the subject; (iv) detecting the level of expression or activity of the NOV1 protein, mRNA, or genomic DNA in the post-administration samples; (v) comparing the level of expression or activity of the NOV1 protein, mRNA, or genomic DNA in the pre-administration sample with the NOV1 protein, mRNA, or genomic DNA in the post administration sample or samples; and (vi) altering the administration of the agent to the subject accordingly. For example, increased administration of the agent may be desirable to increase the expression or activity of NOV1 to higher levels than detected, i.e., to increase the effectiveness of the agent. Alternatively, decreased administration of the agent may be desirable to decrease expression or activity of NOV1 to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOV1 expression or activity. The disorders include cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, and other diseases, disorders and conditions of the like. These methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOV1 expression or activity, by administering to the subject an agent that modulates NOV1 expression or at least one NOV1 activity. Subjects at risk for a disease that is caused or contributed to by aberrant NOV1 expression or activity can be identified by, for example, any or a combination of diagnostic or prognostic assays as described herein. Administration of a prophylactic agent can occur prior to the manifestation of symptoms characteristic of the NOV1 aberrancy, such that a disease or disorder is prevented or, alternatively, delayed in its progression. Depending upon the type of NOV1 aberrancy, for example, a NOV1 agonist or NOV1 antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOV1 expression or activity for therapeutic purposes. The modulatory method of the invention involves contacting a cell with an agent that modulates one or more of the activities of NOV1 protein activity associated with the cell. An agent that modulates NOV1 protein activity can be an agent as described herein, such as a nucleic acid or a protein, a naturally-occurring cognate ligand of a NOV1 protein, a peptide, a NOV1 peptidomimetic, or other small molecule. In one embodiment, the agent stimulates one or more NOV1 protein activity. Examples of such stimulatory agents include active NOV1 protein and a nucleic acid molecule encoding NOV1 that has been introduced into the cell. In another embodiment, the agent inhibits one or more NOV1 protein activity. Examples of such inhibitory agents include antisense NOV1 nucleic acid molecules and anti-NOV1 antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of a NOV1 protein or nucleic acid molecule. In one embodiment, the method involves administering an agent (e.g., an agent identified by a screening assay described herein), or combination of agents that modulates (e.g., up-regulates or down-regulates) NOV1 expression or activity. In another embodiment, the method involves administering a NOV1 protein or nucleic acid molecule as therapy to compensate for reduced or aberrant NOV1 expression or activity.

Stimulation of NOV1 activity is desirable in situations in which NOV1 is abnormally downregulated and/or in which increased NOV1 activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The NOV1 nucleic acids and proteins of the invention are useful in potential prophylactic and therapeutic applications implicated in a variety of disorders including, but not limited to: metabolic disorders, diabetes, obesity, infectious disease, anorexia, cancer-associated cancer, neurodegenerative disorders, Alzheimer's Disease, Parkinson's Disorder, immune disorders, hematopoietic disorders, and the various dyslipidemias, metabolic disturbances associated with obesity, the metabolic syndrome X and wasting disorders associated with chronic diseases and various cancers.

As an example, a cDNA encoding the NOV1 protein of the invention may be useful in gene therapy, and the protein may be useful when administered to a subject in need thereof. By way of non-limiting example, the compositions of the invention will have efficacy for treatment of patients suffering from: metabolic disorders, diabetes, obesity, infectious disease, anorexia, cancer-associated cachexia, cancer, neurodegenerative disorders, Alzheimer's Disease, Parkinson's Disorder, immune disorders, hematopoietic disorders, and the various dyslipidemias.

Both the novel nucleic acid encoding the NOV1 protein, and the NOV1 protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

Identification of Individuals Carrying SNPS

Individuals carrying polymorphic alleles of the invention may be detected at either the DNA, the RNA, or the protein level using a variety of techniques that are well known in the art. Strategies for identification and detection are described in e.g., EP 730,663, EP 717,113, and PCT US97/02102. The present methods usually employ pre-characterized polymorphisms. That is, the genotyping location and nature of polymorphic forms present at a site have already been determined. The availability of this information allows sets of probes to be designed for specific identification of the known polymorphic forms.

Many of the methods described below require amplification of DNA from target samples. This can be accomplished by e.g., PCR. See generally PCR Technology: Principles and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Mattila et al., Nucleic Acids Res. 19, 4967 (1991); Eckert et al., PCR Methods and Applications 1, 17 (1991); PCR (eds. McPherson et al., IRL Press, Oxford); and U.S. Pat. No. 4,683,202.

The phrase “recombinant protein” or “recombinantly produced protein” refers to a peptide or protein produced using non-native cells that do not have an endogenous copy of DNA able to express the protein. In particular, as used herein, a recombinantly produced protein relates to the gene product of a polymorphic allele, e.g., a “polymorphic protein” containing an altered amino acid at the site of translation of the nucleotide polymorphism. The cells produce the protein because they have been genetically altered by the introduction of the appropriate nucleic acid sequence. The recombinant protein will not be found in association with proteins and other subcellular components normally associated with the cells producing the protein. The terms “protein” and “polypeptide” are used interchangeably herein.

The phrase “substantially purified” or “isolated” when referring to a nucleic acid, peptide or protein, means that the chemical composition is in a milieu containing fewer, or preferably, essentially none, of other cellular components with which it is naturally associated. Thus, the phrase “isolated” or “substantially pure” refers to nucleic acid preparations that lack at least one protein or nucleic acid normally associated with the nucleic acid in a host cell. It is preferably in a homogeneous state although it can be in either a dry or aqueous solution. Purity and homogeneity are typically determined using analytical chemistry techniques such as gel electrophoresis or high performance liquid chromatography. Generally, a substantially purified or isolated nucleic acid or protein will comprise more than 80% of all macromolecular species present in the preparation. Preferably, the nucleic acid or protein is purified to represent greater than 90% of all macromolecular species present. More preferably the nucleic acid or protein is purified to greater than 95%, and most preferably the nucleic acid or protein is purified to essential homogeneity, wherein other macromolecular species are not detected by conventional analytical procedures.

The genomic DNA used for the diagnosis may be obtained from any nucleated cells of the body, such as those present in peripheral blood, urine, saliva, buccal samples, surgical specimen, and autopsy specimens. The DNA may be used directly or may be amplified enzymatically in vitro through use of PCR (Saiki et al. Science 239:487-491 (1988)) or other in vitro amplification methods such as the ligase chain reaction (LCR) (Wu and Wallace Genomics 4:560-569 (1989)), strand displacement amplification (SDA) (Walker et al. Proc. Natl. Acad. Sci. U.S.A. 89:392-396 (1992)), self-sustained sequence replication (3SR) (Fahy et al. PCR Methods P&J& 1:25-33 (1992)), prior to mutation analysis.

The method for preparing nucleic acids in a form that is suitable for mutation detection is well known in the art. A “nucleic acid” is a deoxyribonucleotide or ribonucleotide polymer in either single-or double-stranded form, including known analogs of natural nucleotides unless otherwise indicated. The term “nucleic acids”, as used herein, refers to either DNA or RNA. “Nucleic acid sequence” or “polynucleotide sequence” refers to a single-stranded sequence of deoxyribonucleotide or ribonucleotide bases read from the 5′ end to the 3′ end. The direction of 5′ to 3′ addition of nascent RNA transcripts is referred to as the transcription direction; sequence regions on the DNA strand having the same sequence as the RNA and which are beyond the 5′ end of the RNA transcript in the 5′ direction are referred to as “upstream sequences”; sequence regions on the DNA strand having the same sequence as the RNA and which are beyond the 3′ end of the RNA transcript in the 3′ direction are referred to as “downstream sequences”. The term includes both self-replicating plasmids, infectious polymers of DNA or RNA and nonfunctional DNA or RNA. The complement of any nucleic acid sequence of the invention is understood to be included in the definition of that sequence. “Nucleic acid probes” may be DNA or RNA fragments.

The detection of polymorphisms in specific DNA sequences, can be accomplished by a variety of methods including, but not limited to, restriction-fragment-length-polymorphism detection based on allele-specific restriction-endonuclease cleavage (Kan and Dozy Lancet ii:910-912 (1978)), hybridization with allele-specific oligonucleotide probes (Wallace et al. Nucl. Acids Res. 6:3543-3557 (1978)), including immobilized oligonucleotides (Saiki et al. Proc. Natl. Acad. SCI. USA, 86:6230-6234 (1969)) or oligonucleotide arrays (Maskos and Southern Nucl. Acids Res 21:2269-2270 (1993)), allele-specific PCR (Newton et al. Nucl Acids Res 17:2503-_{—}2516 (1989)), mismatch-repair detection (MRD) (Faham and Cox Genome Res 5:474-482 (1995)), binding of MutS protein (Wagner et al. Nucl Acids Res 23:3944-3948 (1995), denaturing-gradient gel electrophoresis (DGGE) (Fisher and Lerman et al. Proc. Natl. Acad. Sci. U.S.A. 80:1579-1583 (1983)), single-strand-conformation-polymorphism detection (Orita et al. Genomics 5:874-879 (1983)), RNAse cleavage at mismatched base-pairs (Myers et al. Science 230:1242 (1985)), chemical (Cotton et al. Proc. Natl. w Sci. U.S.A, 8Z4397-4401 (1988)) or enzymatic (Youil et al. Proc. Natl. Acad. Sci. U.S.A. 92:87-91 (1995)) cleavage of heteroduplex DNA, methods based on allele specific primer_extension (Syvanen et al. Genomics 8:684-692 (1990)), genetic bit analysis (GBA) (Nikiforov et al. &&I Acids 22:4167-4175 (1994)), the oligonucleotide-ligation assay (OLA) (Landegren et al. Science_{—}241:1077 (1988)), the allele-specific ligation chain reaction (LCR) (Barrany Proc. Natl. Acad. Sci. U.S.A. 88:189-193 (1991)), gap-LCR (Abravaya et al. Nucl Acids Res 23:675-682 (1995)), radioactive and/or fluorescent DNA sequencing using standard procedures well known in the art, and peptide nucleic acid (PNA) assays (Orum et al., Nucl. Acids Res, 21:5332-5356 (1993); Thiede et al., Nucl. Acids Res. 24:983-984 (1996)).

“Specific hybridization” or “selective hybridization” refers to the binding, or duplexing, of a nucleic acid molecule only to a second particular nucleotide sequence to which the nucleic acid is complementary, under suitably stringent conditions when that sequence is present in a complex mixture (e.g., total cellular DNA or RNA). “Stringent conditions” are conditions under which a probe will hybridize to its target subsequence, but to no other sequences. Stringent conditions are sequence-dependent and are different in different circumstances. Longer sequences hybridize specifically at higher temperatures than shorter ones. Generally, stringent conditions are selected such that the temperature is about 5° C. lower than the thermal melting point (Tm) for the specific sequence to which hybridization is intended to occur at a defined ionic strength and pH. The Tm is the temperature (under defined ionic strength, pH, and nucleic acid concentration) at which 50% of the target sequence hybridizes to the complementary probe at equilibrium. Typically, stringent conditions include a salt concentration of at least about 0.01 to about 1.0 M Na ion concentration (or other salts), at pH 7.0 to 8.3. The temperature is at least about 30° C. for short probes (e.g., 10 to 50 nucleotides). Stringent conditions can also be achieved with the addition of destabilizing agents such as formamide. For example, conditions of 5×SSPE (750 mM NaCl, 50 mM NaPhosphate, 5 mM EDTA, pH 7.4) and a temperature of 25-30° C. are suitable for allele-specific probe hybridization.

“Complementary” or “target” nucleic acid sequences refer to those nucleic acid sequences which selectively hybridize to a nucleic acid probe. Proper annealing conditions depend, for example, upon a probe's length, base composition, and the number of mismatches and their position on the probe, and must often be determined empirically. For discussions of nucleic acid probe design and annealing conditions, see, for example, Sambrook et al., or Current Protocols in Molecular Biology, F. Ausubel et al., ed., Greene Publishing and Wiley-Interscience, New York (1987).

A perfectly matched probe has a sequence perfectly complementary to a particular target sequence. The test probe is typically perfectly complementary to a portion of the target sequence. A “polymorphic” marker or site is the locus at which a sequence difference occurs with respect to a reference sequence. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The reference allelic form may be, for example, the most abundant form in a population, or the first allelic form to be identified, and other allelic forms are designated as alternative, variant or polymorphic alleles. The allelic form occurring most frequently in a selected population is sometimes referred to as the “wild type” form, and herein may also be referred to as the “reference” form. Diploid organisms may be homozygous or heterozygous for allelic forms. A diallelic polymorphism has two distinguishable forms (e.g., base sequences), and a triallelic polymorphism has three such forms.

As used herein an “oligonucleotide” is a single-stranded nucleic acid ranging in length from 2 to about 60 bases. Oligonucleotides are often synthetic but can also be produced from naturally occurring polynucleotides. A probe is an oligonucleotide capable of binding to a target nucleic acid of a complementary sequence through one or more types of chemical bonds, usually through complementary base pairing via hydrogen bond formation. Oligonucleotides probes are often between 5 and 60 bases, and, in specific embodiments, may be between 10-40, or 15-30 bases long. An oligonucleotide probe may include natural (e.g. A, G, C, or T) or modified bases (7-deazaguanosine, inosine, etc.). In addition, the bases in an oligonucleotide probe may be joined by a linkage other than a phosphodiester bond, such as a phosphoramidite linkage or a phosphorothioate linkage, or they may be peptide nucleic acids in which the constituent bases are joined by peptide bonds rather than by phosphodiester bonds, so long as it does not interfere with hybridization.

As used herein, the term “primer” refers to a single-stranded oligonucleotide which acts as a point of initiation of template-directed DNA synthesis under appropriate conditions (e.g., in the presence of four different nucleoside triphosphates and a polymerization agent, such as DNA polymerase, RNA polymerase or reverse transcriptase) in an appropriate buffer and at a suitable temperature. The appropriate length of a primer depends on the intended use of the primer, but typically ranges from 15 to 30 nucleotides. Short primer molecules generally require cooler temperatures to form sufficiently stable hybrid complexes with the template. A primer need not be perfectly complementary to the exact sequence of the template, but should be sufficiently complementary to hybridize with it. The term “primer site” refers to the sequence of the target DNA to which a primer hybridizes. The term “primer pair” refers to a set of primers including a 5′ (upstream) primer that hybridizes with the 5′ end of the DNA sequence to be amplified and a 3′ (downstream) primer that hybridizes with the complement of the 3′ end of the sequence to be amplified.

DNA fragments can be prepared, for example, by digesting plasmid DNA, or by use of PCR. Oligonucleotides for use as primers or probes are chemically synthesized by methods known in the field of the chemical synthesis of polynucleotides, including by way of non-limiting example the phosphoramidite method described by Beaucage and Carruthers, Tetrahedron Lett 22:1859-1862 (1981) and the triester method provided by Matteucci, et al., J. Am. Chem. Soc. 103:3185 (1981) both incorporated herein by reference. These syntheses may employ an automated synthesizer, as described in Needham-VanDevanter, D. R., et al., Nucleic Acids Res. 12:61596168 (1984). Purification of oligonucleotides may be carried out by either native acrylamide gel electrophoresis or by anion-exchange HPLC as described in Pearson, J. D. and Regnier, F. E., J. Chrom, 255:137-149 (1983). A double stranded fragment may then be obtained, if desired, by annealing appropriate complementary single strands together under suitable conditions or by synthesizing the complementary strand using a DNA polymerase with an appropriate primer sequence. Where a specific sequence for a nucleic acid probe is given, it is understood that the complementary strand is also identified and included. The complementary strand will work equally well in situations where the target is a double-stranded nucleic acid.

The sequence of the synthetic oligonucleotide or of any nucleic acid fragment can be can be obtained using either the dideoxy chain termination method or the Maxam-Gilbert method (see Sambrook et al. Molecular Cloning—a Laboratory Manual (2nd Ed.), Vols. 1-3, Cold Spring Harbor Laboratory, Cold Spring Harbor, N.Y., (1989), which is incorporated herein by reference. This manual is hereinafter referred to as “Sambrook et al.”; Zyskind et al., (1988)). Recombinant DNA Laboratory Manual, (Acad. Press, New York). Oligonucleotides useful in diagnostic assays are typically at least 8 consecutive nucleotides in length, and may range upwards of 18 nucleotides in length to greater than 100 or more consecutive nucleotides.

Another aspect of the invention pertains to isolated antisense nucleic acid molecules that are hybridizable to or complementary to the nucleic acid molecule comprising the SNP-containing nucleotide sequences of the invention, or fragments, analogs or derivatives thereof. An “antisense” nucleic acid comprises a nucleotide sequence that is complementary to a “sense” nucleic acid encoding a protein, e.g., complementary to the coding strand of a double-stranded cDNA molecule or complementary to an mRNA sequence. In specific aspects, antisense nucleic acid molecules are provided that comprise a sequence complementary to at least about 10, about 25, about 50, or about 60 nucleotides or an entire SNP coding strand, or to only a portion thereof.

In one embodiment, an antisense nucleic acid molecule is antisense to a “coding region” of the coding strand of a polymorphic nucleotide sequence of the invention. The term “coding region” refers to the region of the nucleotide sequence comprising codons which are translated into amino acid. In another embodiment, the antisense nucleic acid molecule is antisense to a “noncoding region” of the coding strand of a nucleotide sequence of the invention. The term “noncoding region” refers to 5′ and 3′ sequences which flank the coding region that are not translated into amino acids (i.e., also referred to as 5′ and 3′ untranslated regions).

Given the coding strand sequences disclosed herein, antisense nucleic acids of the invention can be designed according to the rules of Watson and Crick or Hoogsteen base pairing. For example, the antisense nucleic acid molecule can generally be complementary to the entire coding region of an mRNA, but more preferably as embodied herein, it is an oligonucleotide that is antisense to only a portion of the coding or noncoding region of the mRNA. An antisense oligonucleotide can range in length between about 5 and about 60 nucleotides, preferably between about 10 and about 45 nucleotides, more preferably between about 15 and 40 nucleotides, and still more preferably between about 15 and 30 in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used.

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following section).

The antisense nucleic acid molecules of the invention are typically administered to a subject or generated in situ such that they hybridize with or bind to cellular mRNA and/or genomic DNA encoding a polymorphic protein to thereby inhibit expression of the protein, e.g., by inhibiting transcription and/or translation. The hybridization can be by conventional nucleotide complementary to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids Res 15: 6625-6641). The antisense nucleic acid molecule can also comprise a 2′-o-methylribonucleotide (Inoue et al. (1987) Nucleic Acids Res 15: 6131-6148) or a chimeric RNA-DNA analogue (Inoue et al. (1987) FEBS Lett 215: 327-330).

The following terms are used to describe the sequence relationships between two or more nucleic acids or polynucleotides: “reference sequence”, “comparison window”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA or gene sequence given in a sequence listing, or may comprise a complete cDNA or gene sequence. Optimal alignment of sequences for aligning a comparison window may, for example, be conducted by the local homology algorithm of Smith and Waterman Adv. AppI. Math, 2482 (1981), by the homology alignment algorithm of Needleman and Wunsch J. Mol. Biol. 48:443 (1970), by the search for similarity method of Pearson and Lipman Proc. Natl. Acad. Sci. U.S.A. 852444 (1988), or by computerized implementations of these algorithms (for example, GAP, BESTFIT, FASTA, and TFASTA in the Wisconsin Genetics Software Package Release 7.0, Genetics Computer Group, 575 Science Dr., Madison, Wis.).

Techniques for nucleic acid manipulation of the nucleic acid sequences harboring the cSNPs of the invention, such as subcloning nucleic acid sequences encoding polypeptides into expression vectors, labeling probes, DNA hybridization, and the like, are described generally in Sambrook et al. The phrase “nucleic acid sequence encoding” refers to a nucleic acid which directs the expression of a specific protein, peptide or amino acid sequence. The nucleic acid sequences include both the DNA strand sequence that is transcribed into RNA and the RNA sequence that is translated into protein, peptide or amino acid sequence. The nucleic acid sequences include both the full length nucleic acid sequences disclosed herein as well as non-full length sequences derived from the full length protein. It being further understood that the sequence includes the degenerate codons of the native sequence or sequences which may be introduced to provide codon preference in a specific host cell. Consequently, the principles of probe selection and array design can readily be extended to analyze more complex polymorphisms (see EP 730,663). For example, to characterize a triallelic SNP polymorphism, three groups of probes can be designed tiled on the three polymorphic forms as described above. As a further example, to analyze a diallelic polymorphism involving a deletion of a nucleotide, one can tile a first group of probes based on the undeleted polymorphic form as the reference sequence and a second group of probes based on the deleted form as the reference sequence.

For assays of genomic DNA, virtually any biological convenient tissue sample can be used. Suitable samples include whole blood, semen, saliva, tears, urine, fecal material, sweat, buccal, skin and hair. Genomic DNA is typically amplified before analysis. Amplification is usually effected by PCR using primers flanking a suitable fragment e.g., of 50-500 nucleotides containing the locus of the polymorphism to be analyzed. Target is usually labeled in the course of amplification. The amplification product can be RNA or DNA, single stranded or double stranded. If double stranded, the amplification product is typically denatured before application to an array. If genomic DNA is analyzed without amplification, it may be desirable to remove RNA from the sample before applying it to the array. Such can be accomplished by digestion with DNase-free RNase.

Detection of Polymorphisms in a Nucleic Acid Sample

The SNPs disclosed herein can be used to determine which forms of a characterized polymorphism are present in individuals under analysis.

The design and use of allele-specific probes for analyzing polymorphisms is described by e.g., Saiki et al., Nature 324, 163-166 (1986); Dattagupta, EP 235,726, Saiki, WO 89/11548. Allele-specific probes can be designed that hybridize to a segment of target DNA from one individual but do not hybridize to the corresponding segment from another individual due to the presence of different polymorphic forms in the respective segments from the two individuals. Hybridization conditions should be sufficiently stringent that there is a significant difference in hybridization intensity between alleles, and preferably an essentially binary response, whereby a probe hybridizes to only one of the alleles. Some probes are designed to hybridize to a segment of target DNA such that the polymorphic site aligns with a central position (e.g., in a 15-mer at the 7 position; in a 16-mer, at either the 7, 8 or 9 position) of the probe. This design of probe achieves good discrimination in hybridization between different allelic forms.

Allele-specific probes are often used in pairs, one member of a pair showing a perfect match to a reference form of a target sequence and the other member showing a perfect match to a variant form. Several pairs of probes can then be immobilized on the same support for simultaneous analysis of multiple polymorphisms within the same target sequence.

The polymorphisms can also be identified by hybridization to nucleic acid arrays, some examples of which are described in published PCT application WO 95/11995. WO 95/11995 also describes subarrays that are optimized for detection of a variant form of a pre-characterized polymorphism. Such a subarray contains probes designed to be complementary to a second reference sequence, which is an allelic variant of the first reference sequence. The second group of probes is designed by the same principles, except that the probes exhibit complementarity to the second reference sequence. The inclusion of a second group (or further groups) can be particularly useful for analyzing short subsequences of the primary reference sequence in which multiple mutations are expected to occur within a short distance commensurate with the length of the probes (e.g., two or more mutations within 9 to 21 bases).

An allele-specific primer hybridizes to a site on a target DNA overlapping a polymorphism and only primes amplification of an allelic form to which the primer exhibits perfect complementarity. See Gibbs, Nucleic Acid Res. 17 2427-2448 (1989). This primer is used in conjunction with a second primer which hybridizes at a distal site. Amplification proceeds from the two-primers, resulting in a detectable product which indicates the particular allelic form is present. A control is usually performed with a second pair of primers, one of which shows a single base mismatch at the polymorphic site and the other of which exhibits perfect complementarity to a distal site. The single-base mismatch prevents amplification and no detectable product is formed. The method works best when the mismatch is included in the 3′-most position of the oligonucleotide aligned with the polymorphism because this position is most destabilizing to elongation from the primer (see, e.g., WO 93/22456).

Amplification products generated using the polymerase chain reaction can be analyzed by the use of denaturing gradient gel electrophoresis. Different alleles can be identified based on the different sequence-dependent melting properties and electrophoretic migration of DNA in solution. Erlich, ed., PCR Technology, Principles and Applications for DNA Amplification, (W. H. Freeman and Co New York, 1992, Chapter 7).

Alleles of target sequences can be differentiated using single-strand conformation polymorphism analysis, which identifies base differences by alteration in electrophoretic migration of single stranded PCR products, as described in Orita et al., Proc. Nat. Acad. Sci. 86, 2766-2770 (1989). Amplified PCR products can be generated and heated or otherwise denatured, to form single stranded amplification products. Single-stranded nucleic acids may refold or form secondary structures which are partially dependent on the base sequence. The different electrophoretic mobilities of single-stranded amplification products can be related to base-sequence differences between alleles of target sequences.

The genotype of an individual with respect to a pathology suspected of being caused by a genetic polymorphism may be assessed by association analysis. Phenotypic traits suitable for association analysis include diseases that have known but hitherto unmapped genetic components (e.g., agammaglobulinemia, diabetes insipidus, Lesch-Nyhan syndrome, muscular dystrophy, Wiskott-Aldrich syndrome, Fabry's disease, familial hypercholesterolemia, polycystic kidney disease, hereditary spherocytosis, von Willebrand's disease, tuberous sclerosis, hereditary hemorrhagic telangiectasia, familial colonic polyposis, Ehlers-Danlos syndrome, osteogenesis imperfecta, and acute intermittent porphyria).

Phenotypic traits also include symptoms of, or susceptibility to, multifactorial diseases of which a component is or may be genetic, such as autoimmune diseases, high blood pressure, inflammation, cancer, diseases of the nervous system, and infection by pathogenic microorganisms. Some examples of autoimmune diseases include rheumatoid arthritis, multiple sclerosis, diabetes (insulin-dependent and non-independent), systemic lupus erythematosus and Graves disease. Some examples of cancers include cancers of the bladder, brain, breast, colon, esophagus, kidney, oral cavity, ovary, pancreas, prostate, skin, stomach, leukemia, liver, lung, and uterus. Phenotypic traits also include characteristics such as longevity, appearance (e.g., baldness, obesity), strength, speed, endurance, fertility, and susceptibility or receptivity to particular drugs or therapeutic treatments.

Determination of which polymorphic forms occupy a set of polymorphic sites in an individual identifies a set of polymorphic forms that distinguishes the individual. See generally National Research Council, The Evaluation of Forensic DNA Evidence (Eds. Pollard et al., National Academy Press, DC, 1996). Since the polymorphic sites are within a 50,000 bp region in the human genome, the probability of recombination between these polymorphic sites is low. That low probability means the haplotype (the set of all 10 polymorphic sites) set forth in this application should be inherited without change for at least several generations. The more sites that are analyzed the lower the probability that the set of polymorphic forms in one individual is the same as that in an unrelated individual. Preferably, if multiple sites are analyzed, the sites are unlinked. Thus, polymorphisms of the invention are often used in conjunction with polymorphisms in distal genes. Preferred polymorphisms for use in forensics are diallelic because the population frequencies of two polymorphic forms can usually be determined with greater accuracy than those of multiple polymorphic forms at multi-allelic loci.

The capacity to identify a distinguishing or unique set of forensic markers in an individual is useful for forensic analysis. For example, one can determine whether a blood sample from a suspect matches a blood or other tissue sample from a crime scene by determining whether the set of polymorphic forms occupying selected polymorphic sites is the same in the suspect and the sample. If the set of polymorphic markers does not match between a suspect and a sample, it can be concluded (barring experimental error) that the suspect was not the source of the sample. If the set of markers does match, one can conclude that the DNA from the suspect is consistent with that found at the crime scene. If frequencies of the polymorphic forms at the loci tested have been determined (e.g., by analysis of a suitable population of individuals), one can perform a statistical analysis to determine the probability that a match of suspect and crime scene sample would occur by chance.

p(ID) is the probability that two random individuals have the same polymorphic or allelic form at a given polymorphic site. In diallelic loci, four genotypes are possible: AA, AB, BA, and BB. If alleles A and B occur in a haploid genome of the organism with frequencies x and y, the probability of each genotype in a diploid organism are (see WO 95/12607):

Homozygote: p(AA)=x²

Homozygote: p(BB)=y²=(1−x)²

Single Heterozygote: p(AB)=p(BA)=xy=x(1−x)

Both Heterozygotes: p(AB+BA)=2xy=2x(1−x)

The probability of identity at one locus (i.e, the probability that two individuals, picked at random from a population will have identical polymorphic forms at a given locus) is given by the equation:

p(ID)=(x²)²⁺(2xy)²⁺(y²)².

These calculations can be extended for any number of polymorphic forms at a given locus. For example, the probability of identity p(ID) for a 3-allele system where the alleles have the frequencies in the population of x, y and z, respectively, is equal to the sum of the squares of the genotype frequencies:

p(ID)=x⁴⁺(2xy)²⁺(2yz)²⁺(2xz)²⁺z⁴⁺y⁴

In a locus of n alleles, the appropriate binomial expansion is used to calculate p(ID) and p(exc).

The cumulative probability of identity (cum p(ID)) for each of multiple unlinked loci is determined by multiplying the probabilities provided by each locus:

cump(ID)=p(ID1)p(ID2)p(ID3) . . . p(IDn)

The cumulative probability of non-identity for n loci (i.e. the probability that two random individuals will be different at 1 or more loci) is given by the equation:

cump(nonID)=1−cump(ID).

If several polymorphic loci are tested, the cumulative probability of non-identity for random individuals becomes very high (e.g., one billion to one). Such probabilities can be taken into account together with other evidence in determining the guilt or innocence of the suspect.

The object of paternity testing is usually to determine whether a male is the father of a child. In most cases, the mother of the child is known and thus, the mother's contribution to the child's genotype can be traced. Paternity testing investigates whether the part of the child's genotype not attributable to the mother is consistent with that of the putative father. Paternity testing can be performed by analyzing sets of polymorphisms in the putative father and the child.

If the set of polymorphisms in the child attributable to the father does not match the putative father, it can be concluded, barring experimental error, that the putative father is not the real father. If the set of polymorphisms in the child attributable to the father does match the set of polymorphisms of the putative father, a statistical calculation can be performed to determine the probability of coincidental match.

The probability of parentage exclusion (representing the probability that a random male will have a polymorphic form at a given polymorphic site that makes him incompatible as the father) is given by the equation (see WO 95/12607):

p(exc)=xy(1−xy)

where x and y are the population frequencies of alleles A and B of a diallelic polymorphic site. (At a triallelic site p(exc)=xy(1−xy)+yz(1−yz)+xz(1−xz)+3xyz(1−xyz))), where x, y and z and the respective population frequencies of alleles A, B and C). The probability of non-exclusion is:

p(non−exc)=1−p(exc)

The cumulative probability of non-exclusion (representing the value obtained when n loci are used) is thus:

cump(non−exc)=p(non−exc1)p(non−exc2)p(non−exc3) . . . p(non−excn)

The cumulative probability of exclusion for n loci (representing the probability that a random male will be excluded) is:

cump(exc)=1−cump(non−exc).

If several polymorphic loci are included in the analysis, the cumulative probability of exclusion of a random male is very high. This probability can be taken into account in assessing the liability of a putative father whose polymorphic marker set matches the child's polymorphic marker set attributable to his/her father.

The polymorphisms of the invention may contribute to the phenotype of an organism in different ways. Some polymorphisms occur within a protein coding sequence and contribute to phenotype by affecting protein structure. The effect may be neutral, beneficial or detrimental, or both beneficial and detrimental, depending on the circumstances. For example, a heterozygous sickle cell mutation confers resistance to malaria, but a homozygous sickle cell mutation is usually lethal. Other polymorphisms occur in noncoding regions but may exert phenotypic effects indirectly via influence on replication, transcription, and translation. A single polymorphism may affect more than one phenotypic trait. Likewise, a single phenotypic trait may be affected by polymorphisms in different genes. Further, some polymorphisms predispose an individual to a distinct mutation that is causally related to a certain phenotype.

Phenotypic traits include diseases that have known but hitherto unmapped genetic components. Phenotypic traits also include symptoms of, or susceptibility to, multifactorial diseases of which a component is or may be genetic, such as autoimmune diseases, inflammation, cancer, diseases of the nervous system, and infection by pathogenic microorganisms. Some examples of autoimmune diseases include rheumatoid arthritis, multiple sclerosis, diabetes (insulin-dependent and non-independent), systemic lupus erythematosus and Graves disease. Some examples of cancers include cancers of the bladder, brain, breast, colon, esophagus, kidney, leukemia, liver, lung, oral cavity, ovary, pancreas, prostate, skin, stomach and uterus. Phenotypic traits also include characteristics such as longevity, appearance (e.g., baldness, obesity), strength, speed, endurance, fertility, and susceptibility or receptivity to particular drugs or therapeutic treatments.

Correlation is performed for a population of individuals who have been tested for the presence or absence of a phenotypic trait of interest and for polymorphic marker sets. To perform such analysis, the presence or absence of a set of polymorphisms (i.e. a polymorphic set) is determined for a set of the individuals, some of whom exhibit a particular trait, and some of whom exhibit lack of the trait. The alleles of each polymorphism of the set are then reviewed to determine whether the presence or absence of a particular allele is associated with the trait of interest. Correlation can be performed by standard statistical methods and statistically significant correlations between polymorphic form(s) and phenotypic characteristics are noted. For example, it might be found that the presence of allele A1 at polymorphism A correlates with heart disease. As a further example, it might be found that the combined presence of allele A1 at polymorphism A and allele B1 at polymorphism B correlates with increased milk production of a farm animal.

Such correlations can be exploited in several ways. In the case of a strong correlation between a set of one or more polymorphic forms and a disease for which treatment is available, detection of the polymorphic form set in a human or animal patient may justify immediate administration of treatment, or at least the institution of regular monitoring of the patient. Detection of a polymorphic form correlated with serious disease in a couple contemplating a family may also be valuable to the couple in their reproductive decisions. For example, the female partner might elect to undergo in vitro fertilization to avoid the possibility of transmitting such a polymorphism from her husband to her offspring. In the case of a weaker, but still statistically significant correlation between a polymorphic set and human disease, immediate therapeutic intervention or monitoring may not be justified. Nevertheless, the patient can be motivated to begin simple life-style changes (e.g., diet, exercise) that can be accomplished at little cost to the patient but confer potential benefits in reducing the risk of conditions to which the patient may have increased susceptibility by virtue of variant alleles. Identification of a polymorphic set in a patient correlated with enhanced receptiveness to one of several treatment regimes for a disease indicates that this treatment regime should be followed.

For animals and plants, correlations between characteristics and phenotype are useful for breeding for desired characteristics. For example, Beitz et al., U.S. Pat. No. 5,292,639 discuss use of bovine mitochondrial polymorphisms in a breeding program to improve milk production in cows. To evaluate the effect of mtDNA D-loop sequence polymorphism on milk production, each cow was assigned a value of 1 if variant or 0 if wild type with respect to a prototypical mitochondrial DNA sequence at each of 17 locations considered.

The previous section concerns identifying correlations between phenotypic traits and polymorphisms that directly or indirectly contribute to those traits. The present section describes identification of a physical linkage between a genetic locus associated with a trait of interest and polymorphic markers that are not associated with the trait, but are in physical proximity with the genetic locus responsible for the trait and co-segregate with it. Such analysis is useful for mapping a genetic locus associated with a phenotypic trait to a chromosomal position, and thereby cloning gene(s) responsible for the trait. See Lander et al., Proc. Natl. Acad. Sci. (USA) 83, 7353-7357 (1986); Lander et al., Proc. Natl. Acad. Sci. (ISA) 84, 2363-2367 (1987); Donis-Keller et al., Cell 51, 319-337 (1987); Lander et al., Genetics 121, 185-199 (1989)). Genes localized by linkage can be cloned by a process known as directional cloning. See Wainwright, Med. J Australia 159, 170-174 (1993); Collins, Nature Genetics 1, 3-6 (1992) (each of which is incorporated by reference in its entirety for all purposes).

Linkage studies are typically performed on members of a family. Available members of the family are characterized for the presence or absence of a phenotypic trait and for a set of polymorphic markers. The distribution of polymorphic markers in an informative meiosis is then analyzed to determine which polymorphic markers co-segregate with a phenotypic trait. See, e.g., Kerem et al., Science 245, 1073-1080 (1989); Monaco et al., Nature 316, 842 (1985); Yamoka et al., Neurology 40, 222-226 (1990); Rossiter et al., FASEB Journal 5, 21-27 (1991).

Linkage is analyzed by calculation of LOD (log of the odds) values. A lod value is the relative likelihood of obtaining observed segregation data for a marker and a genetic locus when the two are located at a recombination fraction RF, versus the situation in which the two are not linked, and thus segregating independently (Thompson & Thompson, Genetics in Medicine (5th ed, W. B. Saunders Company, Philadelphia, 1991); Strachan, “Mapping the human genome” in The Human Genome (BIOS Scientific Publishers Ltd, Oxford), Chapter 4). A series of likelihood ratios are calculated at various recombination fractions (RF), ranging from RF=0.0 (coincident loci) to RF=0.50 (unlinked). Thus, the likelihood at a given value of RF is: probability of data if loci linked at RF to probability of data if loci unlinked. The computed likelihood is usually expressed as the log₁₀ of this ratio (i.e., a lod score). For example, a lod score of 3 indicates 1000:1 odds against an apparent observed linkage being a coincidence. The use of logarithms allows data collected from different families to be combined by simple addition. Computer programs are available for the calculation of lod scores for differing values of RF (e.g., LIPED, MLINK (Lathrop, Proc. Nat. Acad. Sci. (USA) 81, 3443-3446 (1984)). For any particular lod score, a recombination fraction may be determined from mathematical tables. See Smith et al., Mathematical tables for research workers in human genetics (Churchill, London, 1961); Smith, Ann. Hum. Genet. 32, 127-150 (1968). The value of RF at which the lod score is the highest is considered to be the best estimate of the recombination fraction.

Positive lod score values suggest that the two loci are linked, whereas negative values suggest that linkage is less likely (at that value of RF) than the possibility that the two loci are unlinked. By convention, a combined lod score of +3 or greater (equivalent to greater than 1000:1 odds in favor of linkage) is considered definitive evidence that two loci are linked. Similarly, by convention, a negative lod score of −2 or less is taken as definitive evidence against linkage of the two loci being compared. Negative linkage data are useful in excluding a chromosome or a segment thereof from consideration. The search focuses on the remaining non-excluded chromosomal locations.

The invention further provides transgenic nonhuman animals capable of expressing an exogenous variant gene and/or having one or both alleles of an endogenous variant gene inactivated. Expression of an exogenous variant gene is usually achieved by operably linking the gene to a promoter and optionally an enhancer, and microinjecting the construct into a zygote. See Hogan et al., “Manipulating the Mouse Embryo, A Laboratory Manual,” Cold Spring Harbor Laboratory. (1989). Inactivation of endogenous variant genes can be achieved by forming a transgene in which a cloned variant gene is inactivated by insertion of a positive selection marker. See Capecchi, Science 244, 1288-1292 The transgene is then introduced into an embryonic stem cell, where it undergoes homologous recombination with an endogenous variant gene. Mice and other rodents are preferred animals. Such animals provide useful drug screening systems.

The invention further provides methods for assessing the pharmacogenomic susceptibility of a subject harboring a single nucleotide polymorphism to a particular pharmaceutical compound, or to a class of such compounds. Genetic polymorphism in drug-metabolizing enzymes, drug transporters, receptors for pharmaceutical agents, and other drug targets have been correlated with individual differences based on distinction in the efficacy and toxicity of the pharmaceutical agent administered to a subject. Pharmocogenomic characterization of a subjects susceptibility to a drug enhances the ability to tailor a dosing regimen to the particular genetic constitution of the subject, thereby enhancing and optimizing the therapeutic effectiveness of the therapy.

In cases in which a cSNP leads to a polymorphic protein that is ascribed to be the cause of a pathological condition, method of treating such a condition includes administering to a subject experiencing the pathology the wild type cognate of the polymorphic protein. Once administered in an effective dosing regimen, the wild type cognate provides complementation or remediation of the defect due to the polymorphic protein. The subject's condition is ameliorated by this protein therapy.

A subject suspected of suffering from a pathology ascribable to a polymorphic protein that arises from a cSNP is to be diagnosed using any of a variety of diagnostic methods capable of identifying the presence of the cSNP in the nucleic acid, or of the cognate polymorphic protein, in a suitable clinical sample taken from the subject. Once the presence of the cSNP has been ascertained, and the pathology is correctable by administering a normal or wild-type gene, the subject is treated with a pharmaceutical composition that includes a nucleic acid that harbors the correcting wild-type gene, or a fragment containing a correcting sequence of the wild-type gene. Non-limiting examples of ways in which such a nucleic acid may be administered include incorporating the wild-type gene in a viral vector, such as an adenovirus or adeno associated virus, and administration of a naked DNA in a pharmaceutical composition that promotes intracellular uptake of the administered nucleic acid. Once the nucleic acid that includes the gene coding for the wild-type allele of the polymorphism is incorporated within a cell of the subject, it will initiate de novo biosynthesis of the wild-type gene product. If the nucleic acid is further incorporated into the genome of the subject, the treatment will have long-term effects, providing de novo synthesis of the wild-type protein for a prolonged duration. The synthesis of the wild-type protein in the cells of the subject will contribute to a therapeutic enhancement of the clinical condition of the subject.

A subject suffering from a pathology ascribed to a SNP may be treated so as to correct the genetic defect. (See Kren et al., Proc. Natl. Acad. Sci. USA 96:10349-10354 (1999)). Such a subject is identified by any method that can detect the polymorphism in a sample drawn from the subject. Such a genetic defect may be permanently corrected by administering to such a subject a nucleic acid fragment incorporating a repair sequence that supplies the wild-type nucleotide at the position of the SNP. This site-specific repair sequence encompasses an RNA/DNA oligonucleotide which operates to promote endogenous repair of a subject's genomic DNA. Upon administration in an appropriate vehicle, such as a complex with polyethylenimine or encapsulated in anionic liposomes, a genetic defect leading to an inborn pathology may be overcome, as the chimeric oligonucleotides induces incorporation of the wild-type sequence into the subject's genome. Upon incorporation, the wild-type gene product is expressed, and the replacement is propagated, thereby engendering a permanent repair.

The invention further provides kits comprising at least one allele-specific oligonucleotide as described above. Often, the kits contain one or more pairs of allele-specific oligonucleotides hybridizing to different forms of a polymorphism. In some kits, the allele-specific oligonucleotides are provided immobilized to a substrate. For example, the same substrate can comprise allele-specific oligonucleotide probes for detecting at least 10, 100, 1000 or all of the polymorphisms shown in the Table. Optional additional components of the kit include, for example, restriction enzymes, reverse-transcriptase or polymerase, the substrate nucleoside triphosphates, means used to label (for example, an avidin-enzyme conjugate and enzyme substrate and chromogen if the label is biotin), and the appropriate buffers for reverse transcription, PCR, or hybridization reactions. Usually, the kit also contains instructions for carrying out the hybridizing methods.

Several aspects of the present invention rely on having available the polymorphic proteins encoded by the nucleic acids comprising a SNP of the inventions. There are various methods of isolating these nucleic acid sequences. For example, DNA is isolated from a genomic or cDNA library using labeled oligonucleotide probes having sequences complementary to the sequences disclosed herein.

Such probes can be used directly in hybridization assays. Alternatively probes can be designed for use in amplification techniques such as PCR.

To prepare a cDNA library, mRNA is isolated from tissue such as heart or pancreas, preferably a tissue wherein expression of the gene or gene family is likely to occur. cDNA is prepared from the mRNA and ligated into a recombinant vector. The vector is transfected into a recombinant host for propagation, screening and cloning. Methods for making and screening cDNA libraries are well known, See Gubler, U. and Hoffman, B. J. Gene 25:263-269 (1983) and Sambrook et al.

For a genomic library, for example, the DNA is extracted from tissue and either mechanically sheared or enzymatically digested to yield fragments of about 12-20 kb. The fragments are then separated by gradient centrifugation from undesired sizes and are constructed in bacteriophage lambda vectors. These vectors and phage are packaged in vitro, as described in Sambrook, et al. Recombinant phage are analyzed by plaque hybridization as described in Benton and Davis, Science 196:180-182 (1977). Colony hybridization is carried out as generally described in M. Grunstein et al. Proc. Natl. Acad. Sci. USA. 72:3961-3965 (1975). DNA of interest is identified in either cDNA or genomic libraries by its ability to hybridize with nucleic acid probes, for example on Southern blots, and these DNA regions are isolated by standard methods familiar to those of skill in the art. See Sambrook, et al.

In PCR techniques, oligonucleotide primers complementary to the two 3′ borders of the DNA region to be amplified are synthesized. The polymerase chain reaction is then carried out using the two primers. See PCR Protocols: a Guide to Methods and Applications (Innis, M, Gelfand, D., Sninsky, J. and White, T., eds.), Academic Press, San Diego (1990). Primers can be selected to amplify the entire regions encoding a full-length sequence of interest or to amplify smaller DNA segments as desired. PCR can be used in a variety of protocols to isolate cDNAs encoding a sequence of interest. In these protocols, appropriate primers and probes for amplifying DNA encoding a sequence of interest are generated from analysis of the DNA sequences listed herein. Once such regions are PCR-amplified, they can be sequenced and oligonucleotide probes can be prepared from the sequence.

Once DNA encoding a sequence comprising a cSNP is isolated and cloned, one can express the encoded polymorphic proteins in a variety of recombinantly engineered cells. It is expected that those of skill in the art are knowledgeable in the numerous expression systems available for expression of DNA encoding a sequence of interest. No attempt to describe in detail the various methods known for the expression of proteins in prokaryotes or eukaryotes is made here.

In brief summary, the expression of natural or synthetic nucleic acids encoding a sequence of interest will typically be achieved by operably linking the DNA or cDNA to a promoter (which is either constitutive or inducible), followed by incorporation into an expression vector. The vectors can be suitable for replication and integration in either prokaryotes or eukaryotes. Typical expression vectors contain initiation sequences, transcription and translation terminators, and promoters useful for regulation of the expression of a polynucleotide sequence of interest. To obtain high level expression of a cloned gene, it is desirable to construct expression plasmids which contain, at the minimum, a strong promoter to direct transcription, a ribosome binding site for translational initiation, and a transcription/translation terminator. The expression vectors may also comprise generic expression cassettes containing at least one independent terminator sequence, sequences permitting replication of the plasmid in both eukaryotes and prokaryotes, i.e., shuttle vectors, and selection markers for both prokaryotic and eukaryotic systems. See Sambrook et al.

A variety of prokaryotic expression systems may be used to express the polymorphic proteins of the invention. Examples include E. coli, Bacillus, Streptomyces, and the like.

It is preferred to construct expression plasmids which contain, at the minimum, a strong promoter to direct transcription, a ribosome binding site for translational initiation, and a transcription/translation terminator. Examples of regulatory regions suitable for this purpose in E. coli are the promoter and operator region of the E. coli tryptophan biosynthetic pathway as described by Yanofsky, C., J. Bacterial. 158:1018-1024 (1984) and the leftward promoter of phage lambda as described by A, I. and Hagen, D., Ann. Rev. Genet. 14:399-445 (1980). The inclusion of selection markers in DNA vectors transformed in E. coli is also useful. Examples of such markers include genes specifying resistance to ampicillin, tetracycline, or chloramphenicol. See Sambrook et al. for details concerning selection markers for use in E. coli.

To enhance proper folding of the expressed recombinant protein, during purification from E. coli, the expressed protein may first be denatured and then renatured. This can be accomplished by solubilizing the bacterially produced proteins in a chaotropic agent such as guanidine HCl and reducing all the cysteine residues with a reducing agent such as beta-mercaptoethanol. The protein is then renatured, either by slow dialysis or by gel filtration. See U.S. Pat. No. 4,511,503. Detection of the expressed antigen is achieved by methods known in the art as radioimmunoassay, or Western blotting techniques or immunoprecipitation. Purification from E. coli can be achieved following procedures such as those described in U.S. Pat. No. 4,511,503.

Any of a variety of eukaryotic expression systems such as yeast, insect cell lines, bird, fish, and mammalian cells, may also be used to express a polymorphic protein of the invention. As explained briefly below, a nucleotide sequence harboring a cSNP may be expressed in these eukaryotic systems. Synthesis of heterologous proteins in yeast is well known. Methods in Yeast Genetics, Sherman, F., et al., Cold Spring Harbor Laboratory, (1982) is a well recognized work describing the various methods available to produce the protein in yeast. Suitable vectors usually have expression control sequences, such as promoters, including 3-phosphogtycerate kinase or other glycolytic enzymes, and an origin of replication, termination sequences and the like as desired. For instance, suitable vectors are described in the literature (Botstein, et al., Gene 8:17-24 (1979); Broach, et al., Gene 8:121-133 (1979)).

Two procedures are used in transforming yeast cells. In one case, yeast cells are first converted into protoplasts using zymolyase, lyticase or glusulase, followed by addition of DNA and polyethylene glycol (PEG). The PEG-treated protoplasts are then regenerated in a 3% agar medium under selective conditions. Details of this procedure are given in the papers by J. D. Beggs, Nature (London) 275:104-109 (1978); and Hinnen, A., et al., Proc. Natl. Acad. Sci. USA, 75:1929-1933 (1978). The second procedure does not involve removal of the cell wall. Instead the cells are treated with lithium chloride or acetate and PEG and put on selective plates (Ito, H., et al., J. Bact, 153163-168 (1983)) cells and applying standard protein isolation techniques to the lysates:.

The purification process can be monitored by using Western blot techniques or radioimmunoassay or other standard techniques. The sequences encoding the proteins of the invention can also be ligated to various immunoassay expression vectors for use in transforming cell cultures of, for instance, mammalian, insect, bird or fish origin. Illustrative of cell cultures useful for the production of the polypeptides are mammalian cells. Mammalian cell systems often will be in the form of monolayers of cells although mammalian cell suspensions may also be used. A number of suitable host cell lines capable of expressing intact proteins have been developed in the art, and include the HEK293, BHK21, and CHO cell lines, and various human cells such as COS cell lines, HeLa cells, myeloma cell lines, Jurkat cells, etc. Expression vectors for these cells can include expression control sequences, such as an origin of replication, a promoter (e.g., the CMV promoter, a HSV tk promoter or pgk (phosphoglycerate kinase) promoter), an enhancer (Queen et al. Immunol. Rev, 89:49 (1986)) and necessary processing information sites, such as ribosome binding sites, RNA splice sites, polyadenylation sites (e.g., an SV40 large T Ag poly A addition site), and transcriptional terminator sequences.

Other animal cells are available, for instance, from the American Type Culture Collection Catalogue of Cell Lines and Hybridomas (7th edition, (1992)). Appropriate vectors for expressing the proteins of the invention in insect cells are usually derived from baculovirus. Insect cell lines include mosquito larvae, silkworm, armyworm, moth and Drosophila cell lines such as a Schneider cell line (See Schneider J. Embryol. Exp. Morphol., 27:353-365 (1987). As indicated above, the vector, e.g., a plasmid, which is used to transform the host cell, preferably contains DNA sequences to initiate transcription and sequences to control the translation of the protein. These sequences are referred to as expression control sequences. The term further includes all introns and other DNA sequences spliced from the mRNA transcript, along with variants resulting from alternative splice sites.

A number of types of cells may act as suitable host cells for expression of the protein. Mammalian host cells include, for example, monkey COS cells, Chinese Hamster Ovary (CHO) cells, human kidney 293 cells, human epidermal A43 1 cells, human Col0205 cells, 3T3 cells, CV-1 cells, other transformed primate cell lines, normal diploid cells, cell strains derived from in vitro culture of primary tissue, primary explants, HeLa cells, mouse L cells, BHK, HL-60, U937, HaK or Jurkat cells. Alternatively, it may be possible to produce the protein in lower eukaryotes such as yeast or in prokaryotes such as bacteria. Potentially suitable yeast strains include Saccharomyces cerevisiae, Schizosaccharomyces pombe, Kluyveromyces strains, Candida or any yeast strain capable of expressing heterologous proteins. Potentially suitable bacterial strains include Escherichia coli, Bacillus subtilis, Salmonella typhimurium, or any bacterial strain capable of expressing heterologous proteins. If the protein is made in yeast or bacteria, it may be necessary to modify the protein produced therein, for example by phosphorylation or glycosylation of the appropriate sites, in order to obtain the functional protein.

The protein may also be produced by operably linking the isolated polynucleotide of the invention to suitable control sequences in one or more insect expression vectors, and employing an insect expression system. Materials and methods for baculovirus/insect cell expression systems are commercially available in kit form from, e.g., Invitrogen, San Diego, Calif., U.S.A. (the MaxBac© kit), and such methods are well known in the art, as described in Summers and Smith, Texas Agricultural Experiment Station Bulletin No. 1555 (1987), incorporated herein by reference. As used herein, an insect cell capable of expressing a polynucleotide of the present invention is “transformed.” The protein of the invention may be prepared by culturing transformed host cells under culture conditions suitable to express the recombinant protein.

The polymorphic protein of the invention may also be expressed as a product of transgenic animals, e.g., as a component of the milk of transgenic cows, goats, pigs, or sheep which are characterized by somatic or germ cells containing a nucleotide sequence encoding the protein. The protein may also be produced by known conventional chemical synthesis. Methods for constructing the proteins of the present invention by synthetic means are known to those skilled in the art.

The polymorphic proteins produced by recombinant DNA technology may be purified by techniques commonly employed to isolate or purify recombinant proteins. Recombinantly produced proteins can be directly expressed or expressed as a fusion protein. The protein is then purified by a combination of cell lysis (e.g., sonication) and affinity chromatography. For fusion products, subsequent digestion of the fusion protein with an appropriate proteolytic enzyme releases the desired polypeptide. The polypeptides of this invention may be purified to substantial purity by standard techniques well known in the art, including selective precipitation with such substances as ammonium sulfate, column chromatography, immunopurification methods, and others. See, for instance, R. Scopes, Protein Purification: Principles and Practice, Springer-Verlag: New York (1982), incorporated herein by reference. For example, in an embodiment, antibodies may be raised to the proteins of the invention as described herein. Cell membranes are isolated from a cell line expressing the recombinant protein, the protein is extracted from the membranes and immunoprecipitated. The proteins may then be further purified by standard protein chemistry techniques as described above.

The resulting expressed protein may then be purified from such culture (i.e., from culture medium or cell extracts) using known purification processes, such as gel filtration and ion exchange chromatography. The purification of the protein may also include an affinity column containing agents which will bind to the protein; one or more column steps over such affinity resins as concanavalin A-agarose, heparin-Toyopearl@ or Cibacrom blue 3GA Sepharose B; one or more steps involving hydrophobic interaction chromatography using such resins as phenyl ether, butyl ether, or propyl ether; or immunoaffinity chromatography. Alternatively, the protein of the invention may also be expressed in a form which will facilitate purification. For example, it may be expressed as a fusion protein, such as those of maltose binding protein (MBP), glutathione-S-transferase (GST) or thioredoxin (TRX). Kits for expression and purification of such fusion proteins are commercially available from New England BioLab (Beverly, Mass.), Pharmacia (Piscataway, N.J.) and Invitrogen, respectively. The protein can also be tagged with an epitope and subsequently purified by using a specific antibody directed to such epitope. One such epitope (“Flag”) is commercially available from Kodak (New Haven, Conn.). Finally, one or more reverse-phase high performance liquid chromatography (RP-HPLC) steps employing hydrophobic RP-HPLC media, e.g., silica gel having pendant methyl or other aliphatic groups, can be employed to further purify the protein. Some or all of the foregoing purification steps, in various combinations, can also be employed to provide a substantially homogeneous isolated recombinant protein. The protein thus purified is substantially free of other mammalian proteins and is defined in accordance with the present invention as an “isolated protein.”

The term “antibody” as used herein refers to immunoglobulin molecules and immunologically active portions of immunoglobulin molecules, i.e., molecules that contain an antigen binding site that specifically binds (immunoreacts with) an antigen, such as polymorphic. Such antibodies include, but are not limited to, polyclonal, monoclonal, chimeric, single chain, F_{ab} and F_{(ab′)2} fragments, and an F_{ab} expression library. In a specific embodiment, antibodies to human polymorphic proteins are disclosed.

The phrase “specifically binds to”, “immunospecifically binds to” or is “specifically immunoreactive with”, an antibody when referring to a protein or peptide, refers to a binding reaction which is determinative of the presence of the protein in the presence of a heterogeneous population of proteins and other biological materials. Thus, for example, under designated immunoassay conditions, the specified antibodies bind to a particular protein and do not bind in a significant amount to other proteins present in the sample. Specific binding to an antibody under such conditions may require an antibody that is selected for its specificity for a particular protein. Of particular interest in the present invention is an antibody that binds immunospecifically to a polymorphic protein but not to its cognate wild type allelic protein, or vice versa. A variety of immunoassay formats may be used to select antibodies specifically immunoreactive with a particular protein. For example, solid-phase ELISA immunoassays are routinely used to select monoclonal antibodies specifically immunoreactive with a protein. See Harlow and Lane (1988) Antibodies, a Laboratory Manual, Cold Spring Harbor Publications, New York, for a description of immunoassay formats and conditions that can be used to determine specific immunoreactivity.

Polyclonal and/or monoclonal antibodies that immunospecifically bind to polymorphic gene products but not to the corresponding prototypical or “wild-type” gene products are also provided. Antibodies can be made by injecting mice or other animals with the variant gene product or synthetic peptide. Monoclonal antibodies are screened as are described, for example, in Harlow & Lane, Antibodies, A Laboratory Manual, Cold Spring Harbor Press, New York (1988); Goding, Monoclonal antibodies, Principles and Practice (2d ed.) Academic Press, New York (1986). Monoclonal antibodies are tested for specific immunoreactivity with a variant gene product and lack of immunoreactivity to the corresponding prototypical gene product.

An isolated polymorphic protein, or a portion or fragment thereof, can be used as an immunogen to generate the antibody that binds the polymorphic protein using standard techniques for polyclonal and monoclonal antibody preparation. The full-length polymorphic protein can be used or, alternatively, the invention provides antigenic peptide fragments of polymorphic for use as immunogens. The antigenic peptide of a polymorphic protein of the invention comprises at least 8 amino acid residues of the amino acid sequence encompassing the polymorphic amino acid and encompasses an epitope of the polymorphic protein such that an antibody raised against the peptide forms a specific immune complex with the polymorphic protein. Preferably, the antigenic peptide comprises at least 10 amino acid residues, more preferably at least 15 amino acid residues, even more preferably at least 20 amino acid residues, and most preferably at least 30 amino acid residues. Preferred epitopes encompassed by the antigenic peptide are regions of polymorphic that are located on the surface of the protein, e.g., hydrophilic regions.

For the production of polyclonal antibodies, various suitable host animals (e.g., rabbit, goat, mouse or other mammal) may be immunized by injection with the polymorphic protein. An appropriate immunogenic preparation can contain, for example, recombinantly expressed polymorphic protein or a chemically synthesized polymorphic polypeptide. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmette-Guerin and Corynebacterium parvum, or similar immunostimulatory agents. If desired, the antibody molecules directed against polymorphic proteins can be isolated from the mammal (e.g., from the blood) and further purified by well known techniques, such as protein A chromatography, to obtain the IgG fraction.

The term “monoclonal antibody” or “monoclonal antibody composition”, as used herein, refers to a population of antibody molecules that originates from the clone of a singly hybridoma cell, and that contains only one type of antigen binding site capable of immunoreacting with a particular epitope of a polymorphic protein. A monoclonal antibody composition thus typically displays a single binding affinity for a particular polymorphic protein with which it immunoreacts. For preparation of monoclonal antibodies directed towards a particular polymorphic protein, or derivatives, fragments, analogs or homologs thereof, any technique that provides for the production of antibody molecules by continuous cell line culture may be utilized. Such techniques include, but are not limited to, the hybridoma technique (see Kohler & Milstein, 1975 Nature 256: 495-497); the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

According to the invention, techniques can be adapted for the production of single-chain antibodies specific to a polymorphic protein (see e.g., U.S. Pat. No. 4,946,778). In addition, methodologies can be adapted for the construction of F_{ab} expression libraries (see e.g., Huse, et al., 1989 Science 246: 1275-1281) to allow rapid and effective identification of monoclonal F_{ab} fragments with the desired specificity for a polymorphic protein or derivatives, fragments, analogs or homologs thereof. Non-human antibodies can be “humanized” by techniques well known in the art. See e.g., U.S. Pat. No. 5,225,539. Antibody fragments that contain the idiotypes to a polymorphic protein may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Additionally, recombinant anti-polymorphic protein antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in PCT International Application No. PCT/US86/02269; European Patent Application No. 184,187; European Patent Application No. 171,496; European Patent Application No. 173,494; PCT International Publication No. WO 86/01533; U.S. Pat. No. 4,816,567; European Patent Application No. 125,023; Better et al. (1988) Science 240:1041-1043; Liu et al. (1987) PNAS 84:3439-3443; Liu et al. (1987) J Immunol. 139:3521-3526; Sun et al. (1987) PNAS 84:214-218; Nishimura et al. (1987) Cancer Res 47:999-1005; Wood et al. (1985) Nature 314:446-449; Shaw et al. (1988) J Natl Cancer Inst 80:1553-1559); Morrison(1985) Science 229:1202-1207; Oi et al. (1986) BioTechniques 4:214; U.S. Pat. No. 5,225,539; Jones et al. (1986) Nature 321:552-525; Verhoeyan et al. (1988) Science 239:1534; and Beidler et al. (1988) J Immunol 141:4053-4060.

In one embodiment, methodologies for the screening of antibodies that possess the desired specificity include, but are not limited to, enzyme-linked immunosorbent assay (ELISA) and other immunologically-mediated techniques known within the art.

Anti-polymorphic protein antibodies may be used in methods known within the art relating to the detection, quantitation and/or cellular or tissue localization of a polymorphic protein (e.g., for use in measuring levels of the polymorphic protein within appropriate physiological samples, for use in diagnostic methods, for use in imaging the protein, and the like). In a given embodiment, antibodies for polymorphic proteins, or derivatives, fragments, analogs or homologs thereof, that contain the antibody-derived CDR, are utilized as pharmacologically-active compounds in therapeutic applications intended to treat a pathology in a subject that arises from the presence of the cSNP allele in the subject.

An anti-polymorphic protein antibody (e.g., monoclonal antibody) can be used to isolate polymorphic proteins by a variety of immunochemical techniques, such as immunoaffinity chromatography or immunoprecipitation. An anti-polymorphic protein antibody can facilitate the purification of natural polymorphic protein from cells and of recombinantly produced polymorphic proteins expressed in host cells. Moreover, an anti-polymorphic protein antibody can be used to detect polymorphic protein (e.g., in a cellular lysate or cell supernatant) in order to evaluate the abundance and pattern of expression of the polymorphic protein. Anti-polymorphic antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

### EXAMPLES

### Example 1

### NOV1 Sequence Analysis

TABLE 3
NOV1 Sequence Analysis

| | |
|---|---|
| SEQ ID NO:1 | 3205 bp |

| | |
|---|---|
| NOV 1, | GACAAGAGCTCAGACCTGAGGAGAGTGACTAGCTTCTCTGTGTCCCAGGTGGCCAC |
| CG105201-01 DNA Sequence | |
| CTTCCACTGTGGAAGCTCATGGACTCCATTGGGTCTTCAGGCTTGCGGCAGGGGGA | |
| | AGAAACCCTGAGTTGCTCTGAGGAGGGCTTGCCCGGGCCCTCAGACAGCTCAGAGC |
| | TGGTGCAGGAGTGCCTGCAGCAGTTCAAGGTGACAAGGGCACAGCTACAGCAGATC |
| | CAAGCCAGCCTCTTGGGTTCCATGGAGCAGGCGCTGAGGGGACAGGCCAGCCCTGC |
| | CCCTGCGGTCCGCATGCTGCCTACATACGTGGGGTCCACCCCACATGGCACTGAGC |
| | AAGGAGACTTCGTGGTGCTGGAGCTGGGGGCCACAGGGGCCTCACTGCGTGTTTTG |
| | TGGGTGACTCTAACTCGCATTGAGGGGCATAGGGTGGAGCCCAGAAGCCAGGAGTT |
| | TGTGATCCCCCAAGAGGTGATGCTGGGTGCTGGCCAGCAGCTCTTTGACTTTGCTG |
| | CCCACTGCCTGTCTGAGTTCCTGGATGCGCAGCCTGTGAACAAACAGGGTCTGCAG |
| | CTTGGCTTCAGCTTCTCTTTCCCTTGTCACCAGACGGGCTTGGACAGGAGCACCCT |
| | CATTTCCTGGACCAAAGGTTTTAGGTGCAGTGGTGTGGAAGGCCAGGATGTGGTCC |
| | AGCTGCTGAGAGATGCCATTCGGAGGCAGGGGGCCTACAACATCGACGTGGTTGCT |
| | GTGGTGAACGACACAGTGGGCACCATGATGGGCTGTGAGCCGGGGGTCAGGCCGTG |
| | TGAGGTTGCCCTAGTTGTAGACACGGGCACCAACGCGTGTTACATGGAGGAGGCAC |
| | GGCATGTGGCAGTGCTGGACGAAGACCGGGGCCGCGTCTGCGTCAGCGTCGAGTGG |
| | GGCTCCTTAAGCGATGATGGGGCGCTGGGACCAGTGCTGAGCACCTTCGACCATAC |
| | CCTGGACCATGAGTCCCTGAATCCTGGTGCTCAGAGGTTTGAGAAGATGATCCGAG |
| | GCCTGTACCTGGGTGACCTGGTGCGGCTGGTGCTGGCTCACTTGGCCCGGTGTGGG |
| | GTCCTCTTTCCTGGCTGCACCTCCCCTGCCCTGCTGAGCCAAGGCAGCATCCTCCT |
| | GGAACACGTGGCTGAGATCGAGGAGTGAGTCGGCGAGATGGTGGTTTAGTGGGGGA |
| | TTCTTGGCTTGGAGGAAGCCCATGATACTCTGTTCCCAAGGTAGCCATGGGGCTTT |
| | AGTGGGATGGGGAGCTTCTGGGCTGAGCCCCAAACCACTTCCCTTTCCCCTCCAGC |
| | CCCTCTACTGGGGCAGCCCGTGTCCATGCTATCCTGCAGGACTTGGGCCTGAGCCC |
| | TGGGGCTTCGGATCTTGAGCTTGTGCAGCACGTCTGTGCGGCCGTGTGCACGCGGG |
| | CTCCCCAGCTCTGTGCTGCCGCCCTGGCCGCTCTTCTCTCCTGCCTCCACCACACC |
| | CGGGAGCAACAAACACTCCAGGTTGCTGTGGCCACCGGAGGCCGAGTGTGTGAGCG |
| | GCACCCCAGCTTCTGCAGCGTCCTGCAGGGGACAGTGATGCTCCTGGCCCCGGAAT |
| | GCGATGTCTCCTTAATCCCCTCTGTGGATGGTGGTGGCCGGGGAGTGGCGATGGTG |
| | ACTGCTGTGGCTGCCCGTCTCGCTGCCCACCGGCGCCTGCTGGAGGAGACCCTGGC |
| | CCCATTCCGGTTGAACCATGATCAACTGGCTGCGGTTCAGGCACAGATGCGGAAGG |
| | CCATGGCCAAGGGGCTCCGAGGGGAGGCCTCCTCCCTTCGCATGCTGCCCACTTTC |
| | GTCCGCGCCACCCCTGACGGCAGCGAGCGAGGGGATTTCCTGGCCCTGGACCTCGG |
| | CCGCACGAACTTCCGTGTCCTCCTGCTACGTGTGACCACAGGCGTGCAGATCACCA |
| | GCGAGATCTACTCCATTCCCGAGACTGTGGCCCAGGGTTCTGGGCAGCAGCTCTTT |
| | GACCACATCGTGGACTGCATCGTGGACTTCCAGCAGAAGCAGGGCCTGAGCGGGCA |
| | GAGCCTCCCACTGGGTTTTACCTTCTCCTTCCCATGTAGGCAGCTTGGCCTAGACC |
| | AGGGCATCCTCCTCAACTCCACCAAGGGTTTCAAGGCATCACACTGCGAGGGCCAA |
| | GATGTCGTGACTCTGTTGCGGGAAGCCATCACTCGCAGACAGGCAGTGGAGCTGAA |
| | TGTGGTTGCCATTGTCAATGACACCGTGGGGACCATGATGTCCTGTGGCTATGAGG |
| | ACCCCCGTTGCGAGATAGGCCTCATTGTCGGAACCGGCACCAATGCCTGCTACATG |
| | GAGGAGCTCCGGAATGTGGCGGGCGTGCCTGGGGACTCAGGCCGCATGTGCATCAA |
| | CATGGAGTGGGGCGCCTTTGCGCACGATGGCTCTCTGGCCATGCTCACCACCCGCT |
| | TTGATGCAAGTGTGGACCAGGCGTCCATCAACCCCCGCAAGCAGAGGTTTGAAAAG |
| | ATGATCAGCGGCATGTACCTGGGGGACATCGTCCGCCACATCCTTTTACATTTAAC |
| | CAGCCTTCGCCTTCTCTTCCGGGGCCAGCAGATCCAGCGCCTTCAGACCAGGGACA |
| | TCTTCAAGACCAAGTTCCTCTCTGACATCCAAAGTGACAGCCTGGCCCTGCGCCAG |
| | GTCCGAGCCATCCTAGAGGATCTGGGGCTACCCCTGACCTCAGATGACGCCCTGAT |
| | GGTGCTAGAGGTGTGCCAGGCTGTGTCCCAGAGGGCTGCCCAGCTCTGTGGGCCGG |
| | GTGTAGCTGCCGTCCTGGAGAAGATCCGGGGGAACCGGGCCCTGGAAGAGCTGGCA |
| | CTGTCTGTGGGGGTGGATGGAACGCTCTACAAGCTGCACCCGCGCTTCTCCAGCCT |
| | GGTGGCGGCCACAGTCCGCGAGCTGGCCCCTCGCTGTGTGGTCACGTTCCTGCAGT |
| | CAGAGGATCGGTCCGGCAAAGGTGCGGCCCTCGTCACCGCTGTTGCCTGCCCCCTT |
| | GCGCAGTTGACTCCTGTCTGAGGAAACCTCCACGCTGAGGAGGTCTCCGCCGCAGC |
| | CTTGCTGGAGCCGGGTCGGGGTCTGCCTGTTTCCCAGCCAGGCCCAGCCACCCAGG |
| | ACTCCTGGGACATCCCATGTGTGACCCCTCTGCGGCCATTTGGCCTTGCTCCCTGG |
| | CTTTCCCTGAGAGAAGTAGCACTCAGGTTACCAATATATATATATAATTTATTTAC |
| | AAAAAAAAAAAAA |

| | |
|---|---|
| | ORF Stop: TGA at |
| ORF Start: ATG at 75 | 1146 |
| SEQ ID NO:2 | 357 aa |

| | |
|---|---|
| NOV 1, | MDSIGSSGLRQGEETLSCSEEGLPGPSDSSELVQECLQQFKVTRAQLQQIQASLLG |
| CG105202-01 Amino Acid | |
| Sequence | SMEQALRGQASPAPAVRMLPTYVGSTPHGTEQGDFVVLELGATGASLRVLWVTLTG |
| | IEGHRVEPRSQEFVIPQEVMLGAGQQLFDFAAHCLSEFLDAQPVNKQGLQLGFSFS |
| | FPCHOTGLDRSTLISWTKGFRCSGVEGODVVOLLRDAIRROGAYNIDVVAVVNDTV |
| | GTMMGCEPGVRPCEVGLVVDTGTNACYMEEARHVAVLDEDRGRVCVSVEWGSLSDD |
| | GALGPVLTTFDHTLDHESLNPGAQRFEKMIGGLYLGELVRLVLAHLARCGVLFGGC |
| | TSPALLSQGSILLEHVAEMEE |

For all BLAST data described herein, public nucleotide databases include all GenBank databases and the GeneSeq patent database; and public amino acid databases include the GenBank databases, SwissProt, PDB and PIR.

In all BLAST alignments herein, the “E-value” or “Expect” value is a numeric indication of the probability that the aligned sequences could have achieved their similarity to the BLAST query sequence by chance alone, within the database that was searched. For example, the probability that the subject (“Sbjct”) retrieved from the NOV1 BLAST analysis, e.g., Homo sapiens hexokinase 3 mRNA, matched the Query NOV1 sequence purely by chance is 0.0. The Expect value (E) is a parameter that describes the number of hits one can “expect” to see just by chance when searching a database of a particular size. It decreases exponentially with the Score (S) that is assigned to a match between two sequences. Essentially, the E value describes the random background noise that exists for matches between sequences.

The Expect value is used as a convenient way to create a significance threshold for reporting results. The default value used for blasting is typically set to 0.0001. In BLAST 2.0, the Expect value is also used instead of the P value (probability) to report the significance of matches. For example, an E value of one assigned to a hit can be interpreted as meaning that in a database of the current size one might expect to see one match with a similar score simply by chance. An E value of zero means that one would not expect to see any matches with a similar score simply by chance. See, e.g., http://www.ncbi.nlm.nih.gov/Education/BLASTinfo/. Occasionally, a string of X's or N's will result from a BLAST search. This is a result of automatic filtering of the query for low-complexity sequence that is performed to prevent artifactual hits. The filter substitutes any low-complexity sequence that it finds with the letter “N” in nucleotide sequence (e.g., “NNNNNNNNNNNNN”) or the letter “X” in protein sequences (e.g., “XXXXXXXXX”). Low-complexity regions can result in high scores that reflect compositional bias rather than significant position-by-position alignment. Wootton and Federhen, Methods Enzymol 266:554-571 1996. Other BLAST results include sequences from the Patp database, which is a proprietary database that contains sequences published in patents and patent publications.

[image]

[image]

The SignalP, Psort and/or Hydropathy results predict that NOV1 does not have a signal peptide and is likely to be localized to the cytoplasm with a certainty of 0.4500. In alternative embodiments, a NOV1 polypeptide is located to the microbody (peroxisome) with a certainty of 0.3000, the lysosome (lumen) with a certainty of 0.1646, or the mitochondrial matrix space with a certainty of 0.1000.

The novel nucleic acid encoding the hexokinase 3-like protein of the invention, or fragments thereof, are useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOV1 Antibodies” section below. The disclosed NOV1 protein has multiple hydrophilic regions, each of which can be used as an immunogen. In one embodiment, a contemplated NOV1 epitope is from about amino acids 10 to 50. In another embodiment, a contemplated NOV1 epitope is from about amino acids 70 to 90. In other specific embodiments, contemplated NOV1 epitopes are from about amino acids 110 to 130, 155 to 205, 252 to 260 and 285 to 305.

### Example 2

### SNP Sequence Analysis

TABLE 5
SNP1 Sequence Analysis

| | |
|---|---|
| SEQ ID NO:3 | 3205 bp |

| | |
|---|---|
| Hexokinase 3-like DNA | GACAAGAGCTCAGACCTGAGGAGAGTGACTACCTTCTCTGTGTCCCAGGTGGCCACCTTCCA |
| Sequence | |
| CTGTGGAAGCTCATGGACTCCATTGGGTCTTCAGGGTTGCGGCAGGGGGAAGAAACCCTGAG | |
| | TTGCTCTGAGGAGGGCTTGCCCGGGCCCTCAGACAGCTCAGAGCTGGTGCAGGAGTGCCTGC |
| | ACCAGTTCAAGGTGACAAGGGCACAGCTACAGCAGATCCAAGCCAGCCTCTTGGGTTCCATG |
| | GAGCAGGCGCTGAGGGGACAGGCCAGCCCTGCCCCTGCGGTCCGGATGCTCCCTACATACGT |
| | GGGGTCCACCCCACATGCCACTGAGCAAGGAGACTTCGTGGTGCTGGAGCTGGGGGCCACAG |
| | GGGCCTCACTGCGTGTTTTGTGGGTGACTCTAACTGGCATTGAGGGGCATAGGGTGGAGCCC |
| | AGAAGCCAGGAGTTTGTGATCCCCCAAGAGGTGATGCTGGGTGCTGGCCAGCAGCTCTTTGA |
| | CTTTGCTGCCCACTGCCTGTCTGAGTTCCTGGATGCGCAGCCTGTGAACAAACAGGGTCTGC |
| | AGCTTGGCTTCAGCTTCTCTTTCCCTTCTCACCAGACGGGCTTGGACAGGAGCACCCTCATT |
| | TCCTGGACCAAAGGTTTTAGGTGCAGTGGTGTGGAAGGCCAGGATGTGGTCCAGCTGCTGAG |
| | AGATGCCATTCGGAGGCAGGGGGCCTACAACATCCACGTGGTTGCTGTGGTGAACGACACAG |
| | TGGGCACCATCATGGGCTGTGAGCCGGGGGTCAGGCCGTCTGAGGTTGGGCTAGTTGTAGAC |
| | ACGGGCACCAACGCGTGTTACATGGAGGAGGCACGGCATGTGGCAGTGCTGGACGAAGACCG |
| | GGGCCGCGTCTGCGTCAGCGTCGAGTGGGGCTCCTTAAGCGATGATGGGGCGCTGGGACCAG |
| | TGCTGACCACCTTCGACCATACCCTGGACCATGAGTCCCTGAATCCTGGTGCTCAGAGGTTT |
| | GAGAAGATGATCGGAGGCCTGTACCTGGGTGAGCTGGTGCGGCTGGTGCTGGCTCACTTGGC |
| | CCGGTGTGGGGTCCTCTTTGGTGGCTGCACCTCCCCTGCCCTGCTCAGCCAAGGCAGCATCC |
| | TCCTGGAACACGTGGCTGAGATGGAGGAGTGAGTCGGGGACATGGTGGTTTAGTGGCGGATT |
| | CTTGGCTTGGAGGAAGGGGATGATACTCTGTTCCCAACGTAGCCATGGGGCTTTAGTGGGAT |
| | GGGGAGCTTCTGGGCTGAGCCCCAAACCACTTCCCTTTCCCCTCCAGCCCCTCTACTGGGGC |
| | AGCCCGTGTCCATGCTATCCTGCAGGACTTGGGCCTGAGCCCTGGGGCTTCGGATGTTGAGC |
| | TTGTGCAGCACGTCTGTGCGGCCGTGTGCACGCCGGCTGCCCAGCTCTGTGCTCCCGCCCTG |
| | GCCGCTGTTCTCTCCTGCCTCCAGCACAGCCGGGAGCAACAAACACTCCAGGTTGCTGTGGC |
| | CACCGGAGGCCGAGTGTGTGAGCGGCACCCCAGGTTCTGCAGCGTCCTGCAGGGGACAGTGA |
| | TGCTCCTGGCCCCGGAATGCGATGTCTCCTTAATCCCCTCTGTCGATGGTGCTGGCCGGGGA |
| | GTGGCGATGGTGACTGCTGTGGCTGCCCGTCTGGCTGCCCACCGGCGCCTGCTGGAGGAGAC |
| | CCTGGCCCCATTCCGGTTCAACCATGATCAACTGGCTGCGGTTCAGGCACAGATGCGGAAGG |
| | CCATGGCCAAGGGCCTCCGAGGGGAGGCCTCCTCCCTTCGCATGCTGCCCACTTTCGTCCGG |
| | GCCACCCCTGACGGCAGCGAGCGAGGGGATTTCCTCGCCCTGGACCTCGGGGGCACCAACTT |
| | CCGTGTCCTCCTGGTACGTGTGACCACAGGCGTGCAGATCACCAGCGAGATCTACTCCATTC |
| | CCGAGACTGTGGCCCAGGGTTCTGGGCAGCAGCTCTTTGACCACATCGTGGACTGCATCGTG |
| | GACTTCCAGCAGAAGCAGGGCCTGAGCGGGCAGAGCCTCCCACTGGGTTTTACCTTCTCCTT |
| | CCCATGTAGGCAGCTTGGCCTAGACCAGGGCATCCTCCTGAACTGGACCAAGGGTTTCAAGG |
| | CATCAGACTGCGAGGGCCAAGATGTCGTGAGTCTGTTGCGGGAAGCCATCACTCGCAGACAG |
| | GCAGTCCAGCTGAATGTGGTTGCCATTGTCAATGACACGGTGGQGACCATGATGTCCTGTGC |
| | CTATGAGGACCCCCGTTGCGAGATAGGCCTCATTGTCGGAACCGGCACCAATGCCTGCTACA |
| | TGGAGGAGCTCCGGAATGTGGCGGGCGTGCCTGGGGACTCAGGCCGCATGTGCATCAACATG |
| | CAGTGGGGCGCCTTTGGGGACGATGGCTCTCTGGCCATGCTCAGCACCCGCTTTGATGCAAG |
| | TGTGGACCAGGCGTCCATCAACCCCGGCAAGCAGAGGTTTGAAAAGATGATCAGCGGCATGT |
| | ACCTGGGGGAGATCGTCCGCCACATCCTTTTACATTTAACCAGCCTTGGCGTTCTCTTCCGG |
| | GGCCAGCAGATCCAGCGCCTTCAGACCAGGGACATCTTCAAGACCAAGTTCCTCTCTGAGAT |
| | CGAAAGTGACAGCCTGGCCCTGCGGCAGGTCCGAGCCATCCTAGAGGATCTGGGGCTACCCC |
| | TGACCTCAGATGACGCCCTGATGGTGCTAGAGGTGTGCCAGGCTGTGTCCCAGAGGGCTGCC |
| | CAGCTCTGTGGGGCGGGTGTAGCTGCCGTGGTGGAGAAGATCCGGGGCAACCGGGGCCTGGA |
| | AGAGCTCCCAGTGTCTGTGGGGGTGGATGGAACGCTCTACAAGCTGCACCCGCGCTTCTCCA |
| | GCCTGGTGGCGGCCACAGTGCGGGAGCTGGCCCCTCGCTGTGTGGTCACGTTCCTGCAGTCA |
| | GAGGATGGGTCCGGCAAAGGTGCGGCCCTGGTCACCGCTGTTGCCTGCCGCCTTGCGCAGTT |
| | GACTCGTGTCTGAGGAAACCTCCAGGCTGAGGAGGTCTCCGCCGCAGCCTTGCTGGAGCCGG |
| | GTCGGGGTCTGCCTGTTTCCCAGCCACGCCCAGCCACCCAGGACTCCTGGGACATCCCATGT |
| | GTGACCCCTCTGCGGCCATTTGGCCTTGCTCCCTGGCTTTCCCTGAGAGAAGTAGCACTCAG |
| | GTTAGCAATATATATATATAATTTATTTCAAAAAAAAAAAAA |

| | |
|---|---|
| SEQ ID NO:4 | 357 aa |

| | |
|---|---|
| Hexokinase 3-like | MDSIGSSGLRQGEETLSCSEEGLPGPSDSSELVQECLQQFKVTRAQLQQIQASLLGSMEQAL |
| Amino Acid Sequence | |
| | RGQASPAPAVRMLPTYVGSTPHGTEQGDFVVLELGATGASLRVLWVTLTGIEGHRVEPRSQE |
| | FVIPQEVMLGAGQQLFDFAAHCLSEFLDAQPVNKQGLQLGFSFSFPCHQTGLDRSTLISWTK |
| | GFRCSGVEGQDVVQLLRDAIRRQGAYNIDVVAVVNnTVGTMMGCEPGVRPCEVGLVVDTGTN |
| | ACYMEEARHVAVLDEDRGRVCVSVEWGSLSDDGALGPVLTTFDHTLDHESLNPGAQRFEKMI |
| | GGLYLGELVRLVLAHLARCGVLFGGCTSPALLSQGSILLEHVAEMEE |

| | | | |
|---|---|---|---|
| SEQ ID NO:5 | Nucleotide | Base | Amino Acid |
| | Position: 1630 | Change: | Change: |
| | (underline/bold) | T/C | None (silent |
| | | | mutation |

| | |
|---|---|
| SNP1, Variant | GACAAGAGCTCAGACCTGAGGAGAGTGACTAGCTTCTCTGTGTCCCAGGTGGCCACCTTCCA |
| 12252120, Polymorphic | |
| DNA Sequence morphic | CTGTGGAAGCTCATGGACTCCATTGGGTCTTCAGGGTTGCGGCAGGGGGAAGAAACCCTGAG |
| | TTGCTCTGAGGACGGCTTGCCCGGGCCCTCAGACAGCTCAGAGCTGGTGCAGGAGTGCCTGC |
| | AGCAGTTCAAGGTGACAAGGGCACAGCTACAGCAGATCCAAGCCAGCCTCTTGGGTTCCATG |
| | GAGCAGGCGCTGAGGGGACAGGCCAGCCCTGCCCCTGCGGTCCGGATGCTGCCTACATACGT |
| | GGGGTCCACCCCACATGGCACTGAGCAAGGAGACTTCGTGGTGCTGGAGCTGGGGGCCACAG |
| | GGGCCTCACTGCGTGTTTTGTGGGTGACTCTAACTGGCATTGAGGGGCATAGGGTGGAGCCC |
| | AGAAGCCAGGAGTTTGTGATCCCCCAAGAGGTGATCCTGGGTGCTGGCCAGCAGCTCTTTGA |
| | CTTTGCTGCCCACTGCCTGTCTGAGTTCCTGGATGCGCAGCCTGTGAACAAACAGGGTCTGC |
| | AGCTTGGCTTCAGCTTCTCTTTCCCTTGTCACCAGACGGGCTTGGACAGGAGCACCCTCATT |
| | TCCTGGACCAAAGGTTTTAGGTGCAGTGGTGTGGAAGGCCAGGATGTGGTCCAGCTGCTGAG |
| | AGATGCCATTCGGAGGCAGGGGGCCTACAACATCGACGTGGTTGCTGTGGTGAACGACACAG |
| | TGGGCACCATGATGGGCTGTGAGCCGGGGGTCAGGCCGTGTGAGGTTGGGCTAGTTGTAGAC |
| | ACGGGCACCAACGCGTGTTACATGGAGGAGGCACGGCATGTGGCAGTCCTGGACGAAGACCG |
| | GGGCCGCGTCTGCGTCAGCGTCGAGTGGGGCTCCTTAAGCGATCATGGGGCGCTGGGACCAG |
| | TGCTGACCACCTTCGACCATACCCTGGACCATGAGTCCCTGAATCCTGGTGCTCAGAGGTTT |
| | GAGAAGATGATCGGAGGCCTCTACCTGGGTGAGCTGGTGCGGCTGGTGCTGGCTCACTTGGC |
| | CCGGTGTGGGGTCCTCTTTGGTGGCTGCACCTCCCCTGCCCTGCTGAGCCAAGGCAGCATCC |
| | TCCTGGAACACGTGGCTGAGATGGAGGAGTGAGTCGGGGAGATGGTGGTTTAGTGGGGGATT |
| | CTTGGCTTGCAGGAAGGGGATGATACTCTGTTCCCAAGGTAGCCATGGGGCTTTAGTGGGAT |
| | GGGGAGCTTCTGGGCTGAGCCCCAAACCACTTCCCTTTCCCCTCCAGCCCCTCTACTGGGGC |
| | AGCCCGTGTCCATGCTATCCTGCAGGACTTGGGCCTGAGCCCTGGGGCTTCGGATGTTGAGC |
| | TTGTGCAGCACGTCTGTGCGGCCGTGTGCACGCGCGCTGCCCAGCTCTGTGCTGCCGCCCTC |
| | GCCGCTGTTCTCTCCTGCCTCCAGCACAGCCGGGAGCAACAAACACTCCAGGTTGCTGTGGC |
| | CACCGGAGGCCGAGTGTGTGAGCGGCACCCCAGGTTCTGCAGCGTCCTGCAGGGGACAGTGA |
| | TGCTCCTGCCCCCGGAATGCGATGTCTCCTTAATCCCCTCTGTGGATGGTGGTGGCCGGGGA |
| | GTGGCGATGGTGACTGCCGTGGCTGCCCGTCTGGCTGCCCACCGGCGCCTGCTGGAGGAGAC |
| | CCTGGCCCCATTCCGGTTGAACCATGATCAACTGGCTGCGGTTCAGGCACAGATGCGGAAGG |
| | CCATGGCCAAGGGGCTCCGAGGGGAGGCCTCCTCCCTTCGCATGCTGCCCACTTTCGTCCGG |
| | GCCACCCCTGACGGCAGCGAGCGAGGGGATTTCCTGGCCCTGGACCTCGGGGGCACGAACTT |
| | CCGTGTCCTCCTGGTACGTGTGACCACAGGCGTGCAGATCACCAGCGAGATCTACTCCATTC |
| | CCGAGACTGTGGCCCAGGGTTCTGGGCAGCAGCTCTTTGACCACATCGTGGACTGCATCGTG |
| | GACTTCCAGCAGAACCAGGGCCTGAGCGGGCAGAGCCTCCCACTGGGTTTTACCTTCTCCTT |
| | CCCATGTAGGCAGCTTGGCCTAGACCAGGGCATCCTCCTGAACTCGACCAAGGCTTTCAAGG |
| | CATCAGACTGCGAGGGCCAAGATGTCGTGAGTCTGTTGCGGGAAGCCATCACTCGCAGACAG |
| | GCAGTGGAGCTCAATGTGGTTGCCATTGTCAATGACACGGTGGGGACCATGATGTCCTGTGC |
| | CTATGAGGACCCCCGTTGCGAGATAGGCCTCATTGTCGGAACCGGCACCAATGCCTGCTACA |
| | TGGAGGAGCTCCGGAATGTGGCGGGCGTGCCTGGGCACTCAGGCCGCATGTGCATCAACATG |
| | GAGTGGGGCGCCTTTGCGGACGATGGCTCTCTGGCCATGCTCAGCACCCGCTTTGATGCAAG |
| | TGTGGACCAGGCGTCCATCAACCCCGGCAAGCAGAGGTTTGAAAAGATGATCAGCGGCATGT |
| | ACCTCCGGGAGATCGTCCGCCACATCCTTTTACATTTAACCAGCCTTGCCGTTCTCTTCCGG |
| | GGCCAGCAGATCCAGCGCCTTCAGACCAGGGACATCTTCAAGACCAACTTCCTCTCTGAGAT |
| | CGAAAGTCACAGCCTGGCCCTGCGGCAGGTCCGACCCATCCTAGAGGATCTGGGGCTACCCC |
| | TGACCTCAGATGACGCCCTGATGGTGCTAGAGGTGTGCCAGGCTGTGTCCCAGAGGGCTGCC |
| | CAGCTCTGTGGGGCGGGTGTAGCTGCCGTGGTGGAGAAGATCCGGGGGAACCGGGGCCTGGA |
| | AGAGCTGGCAGTGTCTGTGGGGGTGGATGGAACGCTCTACAAGCTGCACCCGCGCTTCTCCA |
| | GCCTGGTCGCCGCCACAGTGCGGGAGCTGGCCCCTCGCTGTGTGGTCACGTTCCTGCAGTCA |
| | GAGGATGGGTCCGGCAAAGGTGCGGCCCTGGTCACCGCTGTTGCCTGCCGCCTTGCGCAGTT |
| | GACTCGTGTCTGAGGAAACCTCCAGGCTGAGGAGGTCTCCGCCGCAGCCTTGCTGGAGCCGG |
| | GTCGGGGTCTGCCTGTTTCCCAGCCAGGCCCAGCCACCCAGGACTCCTGGGACATCCCATGT |
| | GTGACCCCTCTGCGGCCATTTGGCCTTCCTCCCTGGCTTTCCCTGAGAGAAGTACCACTCAG |
| | GTTAGCAATATATATATATAATTTATTTACAAAAAAAAAAAAA |

TABLE 6
SNP2 Sequence Analysis

| | |
|---|---|
| SEQ ID NO:6 | 2709 bp |

| | |
|---|---|
| SIAT1 DNA Sequence | CTAAAGGTTCCTGTAGGGCGGCACAACCAGGGAGGGCGTGGAAGCTCTGCATCCCTTCTCC |
| | CATACCTTGCTCTACACATCTCTTCATCTGTATCCTCTGCAGCATCCTTGATGATAAACCA |
| | GTAAATATGAGTTTTGATCATCCTGAGAAAAATGGGCCTTGGCCTGCAGACCCAATAAACC |
| | TTCCCTCCCATGGATAATAGTGCTAATTCCTGAGGACCTGAAGGGCCTGCCGCCCCTGGGG |
| | GATTAGCCAGAAGCAGGCTTGTTTTCCTGCTCAGAACAAAGTGACTTCCCTGAACACATCT |
| | TCATTATGATTCACACCAACCTGAAGAAAAAGTTCAGCTGCTGCGTCCTCGTCTTTCTTCT |
| | GTTTGCAGTCATCTGTGTGTGGAAGGAAAAGAAGAAAGGGAGTTACTATGATTCCTTTAAA |
| | TTGCAAACCAAGGAATTCCAGGTGTTAAAGAGTCTGGGGAAATTGGCCATGGGGTCTGATT |
| | CCCAGTCTGTATCCTCAAGCAGCACCCAGGACCCCCACAGGGGCCGCCAGACCCTCGGCAG |
| | TCTCACAGGCCTAGCCAAGGCCAAACCAGAGGCCTCCTTCCAGGTGTGGAACAAGGACAGC |
| | TCTTCCAAAAACCTTATCCCTAGGCTGCAAAAGATCTCGAAGAATTACCTAAGCATGAACA |
| | AGTACAAAGTGTCCTACAAGGGGCCAGGACCAGGCATCAAGTTCAGTGCAGAGGCCCTGCG |
| | CTGCCACCTCCGGGACCATGTGAATGTATCCATGGTAGAGGTCACAGATTTTCCCTTCAAT |
| | ACCTCTGAATGGGAGGGTTATCTGCCCAAGGAGAGCATTAGGACCAAGGCTGGGCCTTGGG |
| | GCAGGTCTGCTGTTGTGTCGTCAGCGGGATCTCTGAAGTCCTCCCAACTACGCAGAGAAAT |
| | CGATGATCATGACGCAGTCCTCAGGTTTAATGGGGCACCCACAGCCAACTTCCAACAAGAT |
| | GTGGGCACAAAAACTACCATTCGCCTGATGAACTCTCAGTTGGTTACCACAGAGAAGCGCT |
| | TCCTCAAAGACAGTTTGTACAATGAAGGAATCCTAATTGTATGGGACCCATCTGTATACCA |
| | CTCAGATATCCCAAAGTGGTACCAGAATCCGGATTATAATTTCTTTAACAACTACAAGACT |
| | TATCCTAAGCTGCACCCCAATCAGCCCTTTTACATCCTCAAGCCCCAGATGCCTTGGGAGC |
| | TATGGGACATTCTTCAAGAAATCTCCCCAGAAGAGATTCAGCCAAACCCCCCATCCTCTGG |
| | GATGCTTGGTATCATCATCATGATGACGCTGTGTGACCAGGTGGATATTTATGAGTTCCTC |
| | CCATCCAAGCGCAAGACTGACGTGTGCTACTACTACCAGAAGTTCTTCGATAGTGCCTGCA |
| | CGATGGGTGCCTACCACCCGCTGCTCTATGAGAAGAATTTGGTGAAGCATCTCAACCAGGG |
| | CACAGATGAGGACATCTACCTGCTTGGAAAAGCCACACTGCCTGGCTTCCGGACCATTCAC |
| | TGCTAAGCACAGGCTCCTCACTCTTCTCCATCAGGCATTAAATGAATGGTCTCTTGCCCAC |
| | CCCAGCCTGGGAAGAACATTTTCCTGAACAATTCCAGCCTGCTCCTTTTACTCTAGGGGCC |
| | TCTGTCAGCAAGACCATGGGGGCTTCAAGAGCCTGTGGTCAGGAAATCAGGTCCAGCCTTC |
| | CCTGTACCCAGACAGTTTATGAGCCCAGAGCCTCCTGCCACACACATGCACACATATCTAG |
| | CATTCTTTCCAGACAGCATCCTCCCCGCCTTCCACCTTGGTAGATGCAAGGTCTATCTCTC |
| | CCATCAGGGCTGCCAAAGCTGGGCTTTGTTTTTCCCAGCAGAATGATGCCATTCTCACAAA |
| | CCAATGCTCTATATTGCTTGAAGTCTGCATCTAAATATTGATTTCACGTTTTAAAGAAATT |
| | CTCTTAAATTACAATTGTGCCCAATGCAGGGTGGCTCTCGGGGGCAAGTAGGTGGTACAGG |
| | GGATTGGAAACATCGTCCGCGCCTCCAGAGAAAAGTTGCTCCCGAGGTCCATGCCCCTGGA |
| | ACGTGTTCCTATCACTCTGGCTGGTTGGGCTGGTCCTTAGACTGGGTGCTTATGATTAAAG |
| | CGTCTTGGTTAGCCCACTTTCCCTCTCCATGTGGAGATGGAAGGTAGAGAAGGATACAGTG |
| | TCTATCCTCAAGTTGCTACGGTTCAGTGAGAGAGGCAGACATCTGAACAGGCAGGTAGGAT |
| | TCAGTGTGCTCAGTGCACTGGGGATTTGGAGAGAGATGGGCTTGCTCTCTCTGTGCACCCA |
| | GGAGGGCCACGCACTTAAAACTGAGTTTGTGGATCAGAGAAGGCTTTATAGCACAGGGGCA |
| | TTCAGATGAGTCTTAGAGGAAGAGAAGAAACATGGCAAGCAGATTACATCTGAGCCGTTTG |
| | AATTGTGTTTTTCTTTCTTCCCATGTTTATTTTCTAAGATCTACCTGAACTTAGAGACTCA |
| | AGATATTTTTTTAGGAAACCTCCTACCCATGTCTGAGGTAGCAAGTGCAGCCTCACGACAC |
| | ATACCAGGCAATCCAGAGCCACAAAACGTGATTCCTCCAGCTCTGCCTGGCCTGACCCTGT |
| | CCTGTCAGCTGGGTTTACATACCAGTCCCATTCTTCCTTTTCAATACCTACCCCCAAATCT |
| | TCTCCTAACCCTAGA |

| | |
|---|---|
| SEQ ID NO: 7 | 406 aa |

| | |
|---|---|
| SIAT1 Amino Acid | MIHTNLKKKFSCCVLVFLLFAVICVWKEKKKGSYYDSFKLQTKEFQVLKSLGKLANGSDSQ |
| Sequence | |
| | SVSSSSTQDPHRGRQTLGSLRGLAKAKPEASFQVWNKDSSSKNLIPRLQKIWKNYLSMNKY |
| | KVSYKGPGPGIKFSAEALRCHLRnHVNVSMVEVTDFPFNTSEWEGYLPKESIRTKAGPWGR |
| | CAVVSSAGSLKSSQLGREIDDHDAVLRFNGAPTANFQQDVGTKTTIRLMNSQLVTTEKRFL |
| | KDSLYNSGILIVWDPSVYHSDIPKWYQNPDYNFFNNYKTYRKLHPNQPFYILKPQMPWELW |
| | DILQEISPEEIQPNPPSSGMLGIIIMMTLCDQVDIYEFLPSKRKTDVCYYYQKFF |

| | | | |
|---|---|---|---|
| | | | Amino |
| | | | Acid |
| | | | Chang |
| | | | e: |
| | | | None |
| | Nucleotide | | (Silent |
| | Position: 1669 | Base Change: | mutati |
| SEQ ID NO: 8 | (underlined/bold) | G/A | on) |

| | |
|---|---|
| SNP2, Variant | CTAAAGGTTCCTGTAGGGCGGCACAACCAGGGAGGGCGTGGAAGCTCTGCATCCCTTCTCC |
| 12252108,Polymorphic | |
| DNA Sequence | CATACCTTGCTCTACACATCTCTTCATCTGTATCCTCTGCAGCATCCTTGATGATAAACCA |
| | GTAAATATGAGTTTTGATCATCCTGAGAAAAATGGGCCTTGGCCTGCAGACCCAATAAACC |
| | TTCCCTCCCATGGATAATAGTGCTAATTCCTGAGGACCTGAAGGGCCTGCCGCCCCTGGGG |
| | TCATTATCATTCACACCAACCTGAAGAAAAAGTTCAGCTGCTGCGTCCTGGTCTTTCTTCT |
| | GTTTGCAGTCATCTGTGTGTGGAAGGAAAAGAAGAAAGGGAGTTACTATGATTCCTTTAAA |
| | TTGCAAACCAAGGAATTCCAGCTGTTAAAGAGTCTGGGGAAATTGGCCATGGGGTCTGATT |
| | CCCAGTCTGTATCCTCAACCAGCACCCAGGACCCCCACAGGGGCCGCCAGACCCTCGGCAG |
| | TCTCAGACGCCTAGCCAAGGCCAAACCACAGGCCTCCTTCCAGGTGTGGAACAAGGACAGC |
| | TCTTCCAAAAACCTTATCCCTAGGCTGCAAAAGATCTGGAAGAATTACCTAAGCATGAACA |
| | AGTACAAAGTGTCCTACAAGGGGCCAGGACCAGGCATCAAGTTCAGTGCAGAGGCCCTGCG |
| | CTGCCACCTCCGGGACCATGTGAATGTATCCATGGTAGAGGTCACAGATTTTCCCTTCAAT |
| | ACCTCTGAATGGGAGGGTTATCTGCCCAAGGAGAGCATTAGGACCAAGGCTGGGCCTTGGG |
| | GCAGGTGTGCTGTTGTCTCGTCAGCGGGATCTCTGAAGTCCTCCCAACTAGGCAGAGAAAT |
| | CCATGATCATGACGCAGTCCTGAGGTTTAATGGGGCACCCACAGCCAACTTCCAACAAGAT |
| | GTGGGCACAAAAACTACCATTCGCCTGATGAACTCTCAGTTGGTTACCACAGAGAAGCGCT |
| | TCCTCAAAGACAGTTTGTACAATGAAGGAATCCTAATTGTATGGGACCCATCTGTATACCA |
| | CTCAGATATCCCAAAGTGGTACCAGAATCCCGATTATAATTTCTTTAACAACTACAAGACT |
| | TATCGTAAGCTGCACCCCAATCAGCCCTTTTACATCCTCAAGCCCCAGATGCCTTGGGAGC |
| | TATGGGACATTCTTCAAGAAATCTCCCCAGAAGAGATTCAGCCAAACCCCCCATCCTCTGG |
| | GATGCTTGGTATCATCATCATGATGACGCTGTGTGACCAGGTGGATATTTATGAGTTCCTC |
| | CCATCCAAGCGCAAGACTGACGTGTGCTACTACTACCAGAAGTTCTTCGATAGTGCCTGCA |
| | CGATGGGTGCCTACCACCCGCTGCTCTATGAGAAGAATTTGGTGAAGCATCTCAACCAGGG |
| | CACAGATGAGGACATCTACCTGCTTGGAAAAGCCACACTGCCTGGCTTCCGGACCATTCAC |
| | TGCTAAGCACACGCTCCTCACTCTTCTCCATCAGGCATTAAATGAATGGTCTCTTGGCCAC |
| | CCCAGCCTGGGAAGAACATTTTCCTGAACAATTCCAGCCTGCTCCTTTTACTCTAGGGGCC |
| | TCTGTCAGCAAGACCATGGGGACTTCAAGAGCCTGTGGTCAGGAAATCAGGTCCAGCCTTC |
| | CCTGTAGCCAGACAGTTTATGAGCCCACAGCCTCCTGCCACACACATGCACACATATCTAG |
| | CATTCTTTCCAGACAGCATCCTCCCCGCCTTCCACCTTGGTAGATGCAAGGTCTATCTCTC |
| | CCATCAGGCCTGCCAAAGCTGGGCTTTGTTTTTCCCAGCAGAATGATGCCATTCTCACAAA |
| | CCAATGCTCTATATTGCTTGAAGTCTGCATCTAAATATTGATTTCACGTTTTAAAGAAATT |
| | CTCTTAAATTACAATTCTGCCCAATGCAGGGTGGCTCTGGGGGGCAAGTAGGTGGTACAGG |
| | GGATTCCAAACATCGTCCGCGCCTCCAGAGAAAAGTTGCTCCCGAGGTCCATGCCCCTGGA |
| | ACGTGTTCCTATCACTCTGGCTGGTTGGGCTGGTCCTTAGACTGGGTGCTTATGATTAAAG |
| | GGTCTTGGTTAGCCCACTTTCCCTCTCCATGTGGAGATGGAAGGTAGAGAAGGATACAGTG |
| | TCTATCCTCAAGTTGCTACGGTTCAGTGAGAGAGGCAGACATCTGAACAGGCAGGTAGGAT |
| | TCAGTGTGCTCACTGCACTGCGGATTTGGAGAGAGATGGGCTTGCTCTCTCTGTGCACCCA |
| | GGAGGGCCACGCACTTAAAACTGAGTTTGTGGATCAGAGAAGGCTTTATAGCACAGGGGCA |
| | TTCAGATGAGTCTTAGAGGAAGAGAAGAAACATGGCAAGCAGATTACATCTGAGCCGTTTG |
| | AATTGTGTTTTTCTTTCTTCCCATGTTTATTTTCTAAGATCTACCTGAACTTAGACACTCA |
| | AGATATTTTTTTAGGAAACCTCCTACCCATGTCTGAGGTAGCAAGTGCAGCCTCACGACAG |
| | ATACCAGGCAATCCAGAGCCACAAAACGTGATTCCTCCAGCTCTGCCTGGCCTGACCCTGT |
| | CCTGTCAGCTGGGTTTACATACCAGTCCCATTCTTCCTTTTCAATACCTACCCCCAAATCT |
| | TCTCCTAACCCTAGA |

TABLE 7
SNP3 Sequence Analysis

| | |
|---|---|
| SEQ ID NO:9 | 3483 bp |

| | |
|---|---|
| PEX6 DNA Sequence | CTGCCTATCGACGCACGACGCAAGATCAATCCGAGGCGCAGCTAACCCCCTCAGAGCAAGT |
| | TCGCGGCACCCGACGCCCCTCCCCTTTTCCTCTGGCCTCCCCTGACGGAAGCGGAAGCGGC |
| | CCTCGCGCACACTAGTCGTCTGGCTCTCTGGCTCCGGAAGCTGTGCTCCTTCACCCTCCTC |
| | GTTGGTGTCCTGTCACCATGGCGCTGGCTGTCTTGCGGGTCCTGGAGCCCTTTCCGACCGA |
| | GACACCCCCGTTGGCAGTGCTGCTGCCACCCGGGGGCCCGTGGCCGGCGGCGGAGCTGGGC |
| | CTGGTGCTGGCCCTGAGGCCTGCAGGGGAGAGCCCGGCAGGGCCGGCGCTGCTGGTGGCAG |
| | CCCTGGAGGGGCCGGACGCGGGCACCGAAGAGCAGGGTCCCGGGCCGCCGCAGCTACTGGT |
| | TAGCCGCGCGCTGCTGCGGCTCCTGGCACTGGGCTCCGGGGCCTGGGTGCGGGCGCGGGCG |
| | GTGCGGCGGCCCCCGGCGCTAGGTTGGGCACTGCTTGGCACCTCGCTGGGGCCTGGGCTCG |
| | GACCGCGAGTCGGGCCGCTGCTGGTGAGGCGCGGAGAGACCCTCCCAGTGCCCGGACCGCG |
| | GGTGCTGGAGACACGGCCGGCGTTGCAAGGGCTGCTGGGCCCAGGGACTCGGCTGGCTGTG |
| | ACTGAGCTCCGCGGGCGGGCCAGACTGTGTCCAGAGTCTGGGCACAGCAGTCGGCCCCCAC |
| | CCCCGCCCGTCGTGTCCTCCTTTGCGGTTTCTGGCACAGTGCGGCGACTCCAGGGAGTTCT |
| | GGGAGGGACTGGAGATTCACTAGGGGTGAGCCGGAGCTGTCTCCGTGGCCTTGGCCTCTTC |
| | CAGGGCGAATGGGTGTGGGTGCCCCAGCCCAGAGAGTCATCGAACACTTCACAGCCGCACT |
| | TGGCTAGGGTGCAGGTCCTAGAACCTCGCTGGGACCTCTCTGATAGACTGGGACCCGGCTC |
| | TGGACCGCTGGGAGAGCCCCTCGCTCACCGACTGGCGCTTGTCCCTGCCACTTTGGCTTTT |
| | AATCTTGGCTGTGACCCCCTCGAAATCGGAGAGCTCAGAATTCAGAGGTACTTCGAAGGCT |
| | CCATCGCCCCTGAACACAAAGGAAGCTGCTCATTGCTGCCTGGGCCTCCATTTGCCAGAGA |
| | GTTACACATCGAAATTGTGTCTTCTCCCCACTACAGTACTAATGGAAATTATGACGGTGTT |
| | CTTTACCGGCACTTTCAGATACCCAGGGTAGTCCAGGAAGGGGATGTTCTATGTGTGCCAA |
| | CAATTGGGCAACTAGACATCCTGGAAGGAAGTCCAGAGAAACTGCCCAGGTGGCGGGAAAT |
| | GTTTTTTAAAGTGAAGAAAACAGTTGGGGAAGCTCCAGATGGACCAGCCAGTGCCTACTTG |
| | GCCGACACCACCCATACCTCCTTGTACATGGTGGGTTCTACCCTGAGCCCTGTTCCATGGC |
| | TCCCTTCAGAGGAATCCACTCTCTGGAGCAGTTTGTCTCCTCCAGGCCTGGAGGCCTTGGT |
| | GTCTGAACTCTGTGCTGTCCTGAAGCCTCGCCTCCAGCCAGGGGGTGCCCTGCTGACAGGA |
| | ACTACCAGTGTCCTTCTACGGGGCCCCCCAGGCTGTGGGAAGACCACAGTAGTTGCTGCTG |
| | CCTGTAGTCACCTTGGGCTCCACTTACTGAAGGTGCCCTGCTCCAGCCTCTGTGCAGAAAG |
| | TAGTGGGGCTGTGGAGACAAAACTGCAGGCCATCTTCTCCCGGGCCCGCCGTTGCCGGCCT |
| | GCAGTCCTGTTGCTCACACCTGTGGACCTTCTGGGCCGGGACCGTGATGGGCTGGGTGACG |
| | ATGCCCGTGTGATGGCTGTGCTGCGTCACCTCCTCCTCAATGAGGACCCCCTCAACAGCTG |
| | CCCTCCCCTCATGGTTGTGGCCACCACAAGCCGGGCCCAGGACCTGCCTGCTGATGTGCAG |
| | ACAGCATTTCCTCATGAGCTCCAGGTGCCTGCTCTGTCAGAGGGGCAGCGGCTCAGCATCC |
| | TGCGGGCCCTCACTGCCCACCTTCCCCTGGGCCAGGAGGTGAACTTGGCACAGCTAGCACG |
| | GCGGTGTGCAGGCTTTGTGGTAGGGGATCTCTATGCCCTTCTGACCCACAGCAGCCGGGCA |
| | GCCTCCACCAGGATCAAGAACTCAGGTTTGGCAGGTGGCTTGACTGAGGAGGATGAGGGGG |
| | AGCTGTGTGCTGCCGGCTTTCCTCTCCTGGCTGAGGACTTTCGGCAGGCACTGGAGCAACT |
| | GCAGACAGCTCACTCCCAGCCCGTTGGAGCCCCCAAGATCCCCTCAGTGTCCTGGCATGAT |
| | GTGGGTGGGCTGCAGGAGCTGAAGAAGGAGATCCTCGAGACCATTCAGCTCCCCCTGGAGC |
| | ACCCTGAGCTACTGAGCCTGGGCCTGAGACGCTCAGGCCTTCTGCTCCATGGGCCCCCTGG |
| | CACCGGCAAGACCCTTCTGGCCAAGCCAGTAGCCACTGAGTGCAGCCTTACCTTCCTCAGC |
| | GTGAAGGGGCCAGAGCTCATTAACATGTATGTGGGCCAAAGTGAGGAGAATGTGCGGGAAG |
| | TGTTTGCCAGGGCCAGGGCTGCAGCTCCATGCATTATCTTCTTTGATGAACTGGACTCTTT |
| | GGCCCCAAGCCGGGGGCCAAGTGGAGATTCTGGAGGAGTGATGGACAGGGTGGTGTCTCAG |
| | CTCCTTGCCGAGCTAGATGGGCTGCACAGCACTCAGGATGTGTTTGTGATTGGAGCCACCA |
| | ACAGACCAGATCTCCTGGACCCTGCCCTTCTGCGGCCTGGCAGATTTGACAAGCTGGTGTT |
| | TGTGGGGGCAAATGAGGACCGGGCCTCCCACCTACGCGTTCTAAGTGCCATCACACGCAAA |
| | TTCAAGCTAGAGCCATCTGTGAGCCTGGTAAACGTGCTAGATTGCTGCCCTCCCCAGCTGA |
| | CGGGCGCGGACCTCTACTCTCTCTGCTCTGATGCTATGACAGCTGCCCTCAAACGCAGGGT |
| | TCATGACCTGGAGGAAGGGCTGGAACAAGGTAGCTCAGCACTGATGCTCACCATGGAGGAC |
| | TTGCTGCAGGCTGCCGCCCGGCTGCAACCCTCAGTCAGTGAGCAGGAGCTGCTCCGGTACA |
| | AGCGCATCCAGCGCAAGTTTGCTGCCTGCTAGGAGCCCCCCAGGGTCTGGGACCCCGCTCA |
| | GCATGGCTGCAGGTACCTTGATAGCCCACAGAGAGATCTGGGAAGGAAGGGCTCCTCCTCA |
| | GGCTGCTGCCAACCCACCTGGAGGCCACCTCCCTCCAGGAGATCCCAGGGTGCAAAGTGGC |
| | ATTGAGACAGCAGCAACAGCTCAAGAGATATCTCCTGCCTACTTGCCCCTCCTTCCAGGCC |
| | GGCTCTAAGAGAAAGGCCCATCTACTCAGGAACAGGGCCAGGGCCTTGGGTTCTGGGGATT |
| | GGGCCCTGAGAGGGCTAGTTCTGTGGCTGAAAATAAAGCATGTCCCGCCCCCTAAAAAAAA |
| | AAAAA |

| | |
|---|---|
| SEQ ID NO. 10 | 980 aa |

| | |
|---|---|
| PEX6 Amino Acid | MALAVLRVLEPFPTETPPLAVLLPPGGPWPAAELGLVLALRPAGESPAGPALLVAALEGPD |
| Sequence | |
| | AGTEEQGPGPPQLLVSRALLRLLALGSGAWVRARAVRRPPALGWALLGTSLGPGLGPRVGP |
| | LLVRRGETLPVPGPRVLETRPALQGLLGPGTRLAVTELRGRARLCPESGDSSRPPPPPVVS |
| | SFAVSGTVRRLQGVLCGTGDSLGVSRSCLRGLGLFQGEWVWVAQARESSNTSQPHLARVQV |
| | LEPRWDLSDRLGPGSGPLGEPLADGLALVPATLAFNLGCDPLEMGELRIQRYLEGSIAPED |
| | KGSCSLLPGPPFARELHIEIVSSPHYSTWGNYDGVLYRHFQIPRVVQEGDVLCVPTIGQVE |
| | ILEGSPEKLPRWREHFFKVKKTVGEAPDGPASAYLADTTHTSLYMVGSTLSPVPWLPSEES |
| | TLWSSLSPPGLEALVSELCAVLKPRLQPGGALLTGTSSVLLRGPPGCGKTTVVAAACSHLG |
| | LHLLKVPCSSLCAESSGAVETKLQAIFSRARRCRPAVLLLTAVDLLGRDRDGLGEDARVMA |
| | VLRHLLLNEDPLNSCPPLMVVATTSRAQDLPADVQTAFPHELEVPALSEGQRLSILEALTA |
| | HLPLGQEVNLAQLARRCAGFVVGDLYALLTHSSRAACTRIKNSGLAGGLTEEDEGELCAAG |
| | FPLLAEDFGQALEQLQTAHSQAVGAPKIPSVSWHDVGGLQEVKKEILETIQLPLEHPELLS |
| | LGLRRSGLLLHGPPGTGKTLLAKAVATECSLTFLSVKGPELINMYVGQSEENVREVFARAR |
| | AAAPCIIFFDELDSLAPSRGRSGDSGGVMDRVVSQLLAELDGLHSTQDVFVIGATNRPDLL |
| | DPALLRPGRFDKLVFVGANEDRASQLRVLSAITRKFKLEPSVSLVNVLDCCPPQLTGADLY |
| | SLCSDAMTAALKRRVHDLEEGLEQGSSALMLTMEDLLQAAARLQP |
| | SVSEQELLRYKRIQRKFAAC |

| | | | | |
|---|---|---|---|---|
| | | | Amino | |
| | | | Acid | |
| | | | Chang | |
| | | | e: | |
| | | | None | |
| | Nucleotide | | (Silent | |
| | Position: 3462 | Base Change: | mutati | |
| SEQ ID NO: 11 | | (underlined/bold) | C/T | on) |

| | |
|---|---|
| SNP3, Variant | CTGCCTATCGAGGCACGACGCAAGATCAATCCGACGCGCAGCTAACCCCCTCAGAGCAAGT |
| 12252123, Polymorphic | |
| DNA Sequence | TCGCGGCACCCGACGCCCCTCCCCTTTTCCTCTGGCCTCCCCTGACGGAAGCGGAAGCGGC |
| | CCTCGCGCACACTAGTCGTCTGGCTCTCTGGCTCCGGAAGCTGTGCTCCTTCACCCTCCTC |
| | GTTCGTGTCCTGTCACCATGGCGCTGGCTCTCTTGCGGGTCCTGGAGCCCTTTCCGACCGA |
| | GACACCCCCGTTGGCAGTGCTCCTGCCACCCGGGGGCCCGTGGCCGGCGCCGGAGCTGGGC |
| | CTGGTGCTGGCCCTGAGGCCTGCAGGGGAGAGCCCGGCACGGCCGGCGCTGCTGGTCGCAG |
| | CCCTGGACGGGCCGGACGCGGGCACCGAAGAGCAGGGTCCCGCGCCGCCGCAGCTACTGCT |
| | TAGCCGCGCGCTGCTGCGGCTCCTGGCACTGGGCTCCGGGGCCTGGGTGCGGGCGCGGCCG |
| | GTGCGGCGGCCCCCGGCCCTAGGTTGGGCACTGCTTGGCACCTCGCTGGGGCCTGGGCTCG |
| | GACCGCGAGTCGGGCCGCTGCTGGTGAGGCGCGGAGAGACCCTCCCAGTGCCCGGACCGCC |
| | GGTGCTGGACACACGGCCGGCGTTGCAACGGCTGCTGGGCCCAGGGACTCGGCTGGCTGTG |
| | ACTGAGCTCCGCGGGCGGGCCAGACTGTGTCCAGAGTCTGGGCACAGCAGTCGGCCCCCAC |
| | CCCCGCCCGTGGTGTCCTCCTTTGCGGTTTCTGGCACAGTGCGGCGACTCCAGGGAGTTCT |
| | GGGAGGGACTGGAGATTCACTAGGGGTGAGCCGGAGCTGTCTCCGTGGCCTTGGCCTCTTC |
| | CAGGGCGAATGGGTGTGGGTGGCCCAGGCCAGAGAGTCATCGAACACTTCACAGCCGCACT |
| | TGGCTAGGGTGCAGGTCCTAGAACCTCGCTGGGACCTCTCTGATAGACTGGGACCCGGCTC |
| | TGGACCGCTGGGAGAGCCCCTCGCTGACGGACTGGCGCTTGTCCCTGCCACTTTGGCTTTT |
| | AATCTTGGCTGTGACCCCCTGGAAATGGGADAGCTCAGAATTCAGAGGTACTTGGAAGGCT |
| | CCATCGCCCCTGAAGACAAAGGAAGCTGCTCATTGCTGCCTGGGCCTCCATTTGCCAGAGA |
| | GTTACACATCGAAATTGTGTCTTCTCCCCACTACAGTACTAATGGAAATTATGACGGTGTT |
| | CTTTACCGGCACTTTCAGATACCCAGGGTAGTCCAGGAAGGGGATGTTCTATGTGTGCCAA |
| | CAATTGGGCAAGTAGAGATCCTGGAAGGAAGTCCAGAGAAACTGCCCAGGTGGCGGGAAAT |
| | GTTTTTTAAAGTGAAGAAAACAGTTGGCGAAGCTCCAGATGGACCACCCAGTGCCTACTTG |
| | GCCGACACCACCCATACCTCCTTGTACATGGTGGGTTCTACCCTGAGCCCTGTTCCATGCC |
| | TCCCTTCAGAGGAATCCACTCTCTGGAGCAGTTTGTCTCCTCCAGGCCTGGAGCCCTTGGT |
| | GTCTGAACTCTGTGCTGTCCTGAAGCCTCGCCTCCAGCCAGGGGGTGCCCTGCTGACAGGA |
| | ACTAGCAGTGTCCTTCTACGGGGCCCCCCAGGCTGTGGGAAGACCACAGTAGTTGCTGCTG |
| | CCTGTAGTCACCTTGGGCTCCACTTACTGAAGGTCCCCTGCTCCAGCCTCTCTGCAGAAAG |
| | TAGTGGGGCTGTGGAGACAAAACTGCAGGCCATCTTCTCCCGGGCCCGCCGTTGCCGGCCT |
| | GCAGTCCTGTTGCTCACACCTGTGGACCTTCTGGGCCGGCACCGTGATGGGCTGGGTGACG |
| | ATGCCCGTGTGATGGCTGTGCTGCGTCACCTCCTCCTCAATGAGGACCCCCTCAACAGCTG |
| | CCCTCCCCTCATGGTTGTGGCCACCACAAGCCGGGCCCAGGACCTGCCTGCTGATGTGCAG |
| | ACAGCATTTCCTCATGAGCTCGAGGTGCCTGCTCTGTCAGAGGGGCAGCGGCTCAGCATCC |
| | TGCGGGCCCTCACTGCCCACCTTCCCCTGGGCCAGGAGGTGAACTTGGCACAGCTAGCACG |
| | GCGGTGTGCAGGCTTTGTGGTAGGGGATCTCTATGCCCTTCTGACCCACAGCAGCCGGGCA |
| | GCCTGCACCAGGATCAAGAACTCAGGTTTGGCAGCTGGCTTGACTGAGGAGGATGAGGGGG |
| | AGCTGTGTGCTGCCGGCTTTCCTCTCCTGGCTGAGGACTTTGGGCAGGCACTGGAGCAACT |
| | GCAGACAGCTCACTCCCAGGCCGTTGGAGCCCCCAAGATCCCCTCAGTGTCCTGGCATGAT |
| | GTGGGTGGGCTGCAGGAGGTGAAGAAGGAGATCCTGGAGACCATTCAGCTCCCCCTGGACC |
| | ACCCTGAGCTACTGAGCCTGGGCCTGAGACGCTCAGGCCTTCTGCTCCATGGGCCCCCTGG |
| | CACCGGCAAGACCCTTCTGGCCAACGCAGTAGCCACTCAGTGCAGCCTTACCTTCCTCAGC |
| | GTGAAGGGGCCAGAGCTCATTAACATGTATGTGGGCCAAAGTGAGGAGAATGTGCGGGAAG |
| | TGTTTGCCAGGGCCAGGGCTGCAGCTCCATGCATTATCTTCTTTGATGAACTGGACTCTTT |
| | GGCCCCAAGCCGGGGGCGAAGTGGAGATTCTGGAGGAGTGATCGACAGGGTGGTGTCTCAG |
| | CTCCTTGCCCAGCTAGATGGGCTGCACAGCACTCAGGATGTGTTTGTGATTGCAGCCACCA |
| | ACAGACCAGATCTCCTGGACCCTGCCCTTCTGCGCCCTGCCAGATTTGACAAGCTGGTGTT |
| | TGTGGGGGCAAATGAGGACCGGGCCTCCCAGCTACGCGTTCTAAGTGCCATCACACGCAAA |
| | TTCAAGCTAGAGCCATCTGTGAGCCTGGTAAACGTGCTAGATTGCTGCCCTCCCCAGCTGA |
| | CGGGCGCGGACCTCTACTCTCTCTGCTCTGATGCTATGACAGCTGCCCTCAAACGCACGGT |
| | TCATGACCTGGAGGAAGGGCTGGAACAAGGTAGCTCAGCACTGATGCTCACCATGGAGGAC |
| | TTGCTGCAGGCTGCCGCCCGGCTGCAACCCTCAGTCAGTGAGCAGGAGCTGCTCCGGTACA |
| | AGCGCATCCAGCGCAAGTTTGCTGCCTGCTAGGAGCCCCCCAGGGTCTGGGACCCCGCTCA |
| | GCATGGCTGCAGGTACCTTGATAGCCCACAGAGAGATCTGGGAAGGAAGGGCTCCTCCTCA |
| | GGCTGCTGCCAACCCACCTGGAGGCCACCTCCCTCCAGGAGATCCCAGGGTGCAAAGTGGC |
| | ATTGAGACAGCAGCAACAGCTCAAGAGATATCTCCTGCCTACTTGCCCCTCCTTCCAGGCC |
| | GGCTCTAAGAGAAAGGCCCATCTACTCAGGAAGAGGGCCAGGGCCTTGGGTTCTGGGGATT |
| | GGGCCCTGAGAGGGCTAGTTCTGTGGCTGAAAATAAAGCATGTCCTGCCCCCTAAAAAAAA |
| | AAAAA |

### Example 3

### Method of SNP Identification

SeqCallingTM Technology: cDNA was derived from various human samples representing multiple tissue types, normal and diseased states, physiological states, and developmental states from different donors. Samples were obtained as whole tissue, cell lines, primary cells or tissue cultured primary cells and cell lines. Cells and cell lines may have been treated with biological or chemical agents that regulate gene expression for example, growth factors, chemokines, steroids. The cDNA thus derived was then sequenced using CuraGen's proprietary SeqCalling technology. Sequence traces were evaluated manually and edited for corrections if appropriate. cDNA sequences from all samples were assembled with themselves and with public ESTs using bioinformatics programs to generate CuraGen's human SeqCalling database of SeqCalling assemblies. Each assembly contains one or more overlapping cDNA sequences derived from one or more human samples. Fragments and ESTs were included as components for an assembly when the extent of identity with another component of the assembly was at least 95% over 50 bp. Each assembly can represent a gene and/or its variants such as splice forms and/or single nucleotide polymorphisms (SNPs) and their combinations.

Variant sequences are included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, however, in the case that a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern for example, alteration in temporal expression, physiological response regulation, cell type expression regulation, intensity of expression, stability of transcribed message.

Method of novel SNP Identification: SNPs are identified by analyzing sequence assemblies using CuraGen's proprietary SNPTool algorithm. SNPTool identifies variation in assemblies with the following criteria: SNPs are not analyzed within 10 base pairs on both ends of an alignment; Window size (number of bases in a view) is 10; The allowed number of mismatches in a window is 2; Minimum SNP base quality (PHRED score) is 23; Minimum number of changes to score an SNP is 2/assembly position. SNPTool analyzes the assembly and displays SNP positions, associated individual variant sequences in the assembly, the depth of the assembly at that given position, the putative assembly allele frequency, and the SNP sequence variation. Sequence traces are then selected and brought into view for manual validation. The consensus assembly sequence is imported into CuraTools along with variant sequence changes to identify potential amino acid changes resulting from the SNP sequence variation. Comprehensive SNP data analysis is then exported into the SNPCalling database.

Method of novel SNP Confirmation: SNPs are confirmed employing a validated method know as Pyrosequencing. Detailed protocols for Pyrosequencing can be found in: Alderbom et al. Determination of Single Nucleotide Polymorphisms by Real-time Pyrophosphate DNA Sequencing. (2000). Genome Research. 10, Issue 8, August. 1249-1265.

In brief, Pyrosequencing is a real time primer extension process of genotyping. This protocol takes double-stranded, biotinylated PCR products from genomic DNA samples and binds them to streptavidin beads. These beads are then denatured producing single stranded bound DNA. SNPs are characterized utilizing a technique based on an indirect bioluminometric assay of pyrophosphate (PPi) that is released from each dNTP upon DNA chain elongation. Following Klenow polymerase-mediated base incorporation, PPi is released and used as a substrate, together with adenosine 5′-phosphosulfate (APS), for ATP sulfurylase, which results in the formation of ATP. Subsequently, the ATP accomplishes the conversion of luciferin to its oxi-derivative by the action of luciferase. The ensuing light output becomes proportional to the number of added bases, up to about four bases. To allow processivity of the method dNTP excess is degraded by apyrase, which is also present in the starting reaction mixture, so that only dNTPs are added to the template during the sequencing. The process has been fully automated and adapted to a 96-well format, which allows rapid screening of large SNP panels.

Method of novel SNP association with a phenotypic trait: The association of a SNP with a defined phenotypic trait is discovered through statistical genetic analysis of the SNP in a population sample of humans in which the phenotypic trait under investigation has been characterized. Such a population may consist of unrelated individuals, or of related individuals such as sibling pairs (including dizygotic or monozygotic twins), offspring & parents, or other familial structures comprised of genetically related individuals. These populations may be ascertained based upon the presence of one or more disease-affected individual(s) within each family, or may be ascertained as an epidemiologic sample representing the entire population. The phenotypic traits may be any observable or measurable characteristic of humans, including but not limited to biochemical assays, assays of physiological function or performance, and clinical measures of growth and development such as body mass index. Specific analytic methods used depend upon the specific family structures, such as QTDT for sibling pairs (reference: Abecasis et al. A General Test of Association for Quantitative Traits in Nuclear Families. Am J Hum Genet (2000) 66:279-292).

### Example 4

### Population, Clinical Measurements and Genotypes

The population providing evidence for the association between the genetic variants and the disease comprised 2400 individuals consisting of 800 dizygotic (DZ) sib-pairs and 400 monozygotic (MZ) sib-pairs. The individuals were all female, ranged in age from approximately 20 to 70 years, and were all of Caucasian ethnicity. Age and zygosity were recorded for every sib-pair, and self-reported zygosity was confirmed by genotyping a standard marker set to confirm 50% or 100% allele sharing by DZ and MZ pairs, respectively.

Clinical measurements were made for 105 traits in categories including asthma and respiratory disease, biochemistry and endocrine function, bone density and osteoporosis, cardiovascular disease, diabetes, hypertension, obesity, immunology, rheumatology, oncology, CNS disorders, and dermatology. Each trait was measured for approximately 80% of the population.

Each trait was standardized to approximate a univariate standard normal distribution. For most traits, this involved calculating the trait mean and standard deviation, then subtracting the mean for each trait score and dividing by the standard deviation to yield a trait with zero mean and unit variance. For some traits, the distribution appeared log-normal, and a log transform was applied prior to the standardization.

Genotypes were measured for each marker for at least 70% of the individuals with a discrepancy rate of 4% or less. Genotyping discrepancies do not increase the false-positive rate of a test, although they do increase the false-negative rate.

Genotyping was performed for two of the SNPs: SNP1 (12252120) and SNP3 (12252123). The results are shown below:

Genotype Results for SNP1:

homozygous major allele CC 390

heterozygous CT 428

homozygous minor allele TT 94

Genotype Results for SNP3:

homozygous CC 940

heterozygous CT 112

homozygous TT 0

Statistical Analysis for Each Marker/Trait Combination

Data Collection

An individual was defined as informative if both the trait value and genotype were available. The total population was then partitioned into three groups: MZ pairs with both sibs informative, DZ pairs having both sibs informative, and unrelateds from both MZ pairs and DZ pairs in which only one sib was informative.

The terms nUnrel, nMZ, and nDZ refer to the number of unrelateds, number of MZ pairs, and number of DZ pairs, respectively; the total number of informative individuals is nUnrel+2 nMZ+2 nDZ.

The allele frequency of the minor allele (a number between 0 and 0.5) was determined as a weighted average in which unrelated individuals had a weight of 1, MZ individuals had a weight of 0.5, and DZ individuals had a weight of 0.75. These weightings account for genotypic correlation within a sib-pair. The markers we tested were all bi-allelic. The frequency of the minor allele, termed A, is denoted p, and the frequency of the major allele, termed allele B, is denoted q and equals 1−p.

Hardy-Weinberg Tests

Hardy-Weinberg equilibrium (HWE) relates genotype frequencies to allele frequencies under general assumptions of an equilibrium population. Violations of HWE may indicate selection against the minor allele and population stratification. Selection against the minor allele occurs when the minor allele detracts from evolutionary fitness and may result in having fewer homozygotes than would be expected by chance.

Population stratification arises when the population being studies is actually a mix of sub-populations with different frequencies of allele A. Stratification results in having more homozygotes than would be expected by chance. Stratification may increase the false-positive and false-negative rates for between-family tests but does not affect within-family tests (see below). Thus, if stratification is indicated, it is preferable to perform only within-family tests.

To perform Hardy-Weinberg tests, one individual was selected at random from each MZ and DZ pair to yield a total of N=nUnrel+nMZ+nDZ unrelated individuals. The counts of individuals with AA, AB, and BB genotypes in this population were termed N(AA), N(AB), and N(BB), respectively, and the allele frequency p was calculated as

p=[N(AA)+0.5N(BB)]/N.

Next, the counts of individuals expected for each genotype under the null hypothesis of HWE were calculated as

n(AA)=p²N

n(AB)=2pqN

n(BB)=q²N

Finally, two test statistics were calculated:

HW1=[N(AA)−n(AA)]²/n(AA)+[N(AB)−n(AB)]²/n(AB)+[N(BB)−n(BB)]²/n(BB) HW2={[N(AA)+N(BB)]−[n(AA)+n(BB)]}²/{n(AA)+n(BB)}+[N(AB)−n(AB)]²/n(AB)

Under the null hypothesis, both HW1 and HW2 follow χ² distributions with 1 degree of freedom. The critical values of χ² for p-values of 0.05 and 0.01 are 3.84 and 6.63 respectively. Values of χ² larger than these indicate a 5% chance or a 1% chance of the HW assumptions being satisfied.

The HW1 test is the standard test, but it is not accurate when the smallest category, typically N(AA), has fewer than 5 individuals. The HW2 test is more robust but can be less sensitive for rare alleles. If there is significant deviation from HWE, the sign of [N(AA)+N(BB)]−[n(AA)+n(BB)] indicates the reason: positive values indicate stratification and negative values indicate selection against the minor allele.

Association Tests

Association tests were based on a genetic model for the marker as a quantitative trait locus (QTL),

X_{fi}=Y_{f}+Y_{fi}+m(G_{fi})

where X_{fi} is the phenotypic value of individual i in family f, Y_{f} represents the contribution to X_{fi} from shared genetic and environmental effects excluding effects from the QTL, Y_{fi} represents the non-shared contributions excluding the QTL, and m(G_{fi}) represents the mean effect from the QTL and depends only on the genotype G_{fi}, with

m(AA)=a−c

m(AB)=d−c

m(BB)=−a−c,

where the constant c is defined as (p−q)a+2pqd.

Instead of testing for the significance of both a and d, we focused on just the additive contribution from the allele to the phenotype by testing the significance of the regression coefficient b in the model

Xᵢ=Yᵢ+a+bpᵢ

where Xᵢ is the phenotypic value for sample i, Yᵢ represents the contributions to the phenotype excluding the QTL for sample i, and pᵢ is the allele frequency for sample i.

Since pᵢ takes a discrete number of values, the tests were performed by calculating the mean and standard error of Xᵢ for each value of pᵢ, then performing a regression test of the binned values to obtain b and its sampling standard deviation s. Under the null hypothesis of no association, b/s follows a standard normal distribution. The p-value for a significant association was calculated from a two-sided test of b/s.

A total of 6 tests of this nature were performed:

Unrelated Xᵢ, and pᵢ are from the unrelated individuals and the MZ pairs. For the unrelateds, each individual yields a single sample of Xᵢ and pᵢ. For the MZ pairs, Xᵢ and pᵢ were taken as the average of the two values. It would be preferable to account for the phenotypic correlation between MZ sibs as part of this test.

Mean Each DZ pair yields a single sample, with Xᵢ and pᵢ equal to the mean phenotypic value and allele frequency of pair i.

Difference Each DZ pair yields a single sample, with Xᵢ and pᵢ equal to the difference in phenotypic value and allele frequency between the first and second sib. This test is robust to stratification.

Non-parametric difference Each DZ pair yields a single sample, with pᵢ equal to the difference in allele frequency between the first and second sib, and Xᵢ equal to 1, 0, or −1 if the phenotypic value of the first sib is greater than, equal to, or less than that of the second sib. This test is like a transmission disequilibrium test (TDT). Like the difference test, it is robust to stratificationl; it is also robust to non-normality and outliers, but is less sensitive to small effects than the difference test.

Total The total test combines the estimates of b from the unrelated, mean, and difference tests, which are statistically independent. A minimum variance estimator of b is built by weighting each of the three tests by the inverse of their sampling variance, and the variance of the combined estimator is the inverse of the sum of the inverse variances of the independent estimates. This test is more sensitive than either of the three independent tests in the absence of stratification, but is not as robust as the difference or non-parametric difference test in the presence of stratification.

Stratification The test statistic for the stratification test is the square of the difference of the estimates of b from the mean and difference tests, normalized by the sum of the variances of the two estimators, follows a χ² distribution with 1 degree of freedom. Large values of the test statistic indicate population stratification and that only the difference test and non-parametric difference test may be robust.

For the mean, difference, and total tests, the term b is related to the parameters of the genetic model as

b=2[a−(p−q)d].

The effect size was reported as the quantity a assuming additive inheritance (d=0), then taking the ratio of a to the standard deviation of the trait value.

We also applied a multiple testing correction by requiring a p-value of less than approximately 10⁻³ for a significant test. The roughly 100 phenotypes tests correspond to approximately 20 independent tests because many of the phenotypes are correlated; this threshold corresponds to an approximate false-positive rate of 2% per marker tested.

### Example 7

### Output Analysis for SNPs

SNP1: 12252120 with Serum Bicarbonate

Marker 12252120 Trait BICARB (Attributes 11 and 85)

| nUnrel 109 | nMZ pairs 259nDZ pairs 544 | |
|---|---|---|
| minor allele | 1 | |
| allele freq | 0.339421 | |
| var(freq) | 0.112107 (est) 0.104375 (DZ+) | 0.123621 (DZ−) |
| trait mean | | |
| unrel 0.318176 | | |
| MZ 0.041768 | | |
| DZ 0.000854 | | |
| tot 0.039017 | | |
| trait var | | |
| Tot 6.936838 | | |
| Gen | 2.623137 (0.378146) | |
| ShEnv | 2.682621 (0.386721) | |
| NShEnv | 1.631079 (0.235133) | |
| corln | 0.575794 | |

| hwTest | N(AA) | N(AB) | N(BB) | N | p | q |
|---|---|---|---|---|---|---|
| Obs: | 390 | 428 | 94 | 912 | 0.66228 | 0.33772 |
| Exp: | 400.02 | 407.96 | 104.02 | | | |
| Delta: | −10.02 | 20.04 | −10.02 | | | |
| ChiSq: | 0.25 | 0.98 | 0.96 | | | |
| | N(AA + BB) | N(AB) | | | | |
| Obs: | 484 | 428 | | | | |
| Exp: | 504.04 | 407.96 | | | | |
| Delta: | −20.04 | 20.04 | | | | |
| ChiSq: | 0.80 | 0.98 | | | | |

| Test | Value | P-Value |
|---|---|---|
| 3-bin chi sq | 2.19954 | 0.138052 |
| 2-bin chi sq | 1.78030 | 0.182112 |

| | | | |
|---|---|---|---|
| unrel | 0.150229 | 0.395672 +/− 0.212697 | pval 0.062849 |
| mean | 0.077167 | 0.203242 +/− 0.177199 | pval 0.251394 |
| diff | 0.155076 | 0.408438 +/− 0.138372 | pyal 0.003160 |
| diffnp | 0.050159 | 0.132109 +/− 0.054779 | pval 0.015880 |
| tot | 0.130699 | 0.344234 +/− 0.097046 | pval 0.000389 |
| strat | −0.077909 | −0.205196 +/− 0.224825 | pval 0.361405 |

SNP3: 12252123 with Body Mass Index, TFATM and WAIST

Marker 12252123 Trait TFATM (Attributes 1 and 47)

| | | |
|---|---|---|
| nUnrel 254 | nMZ pairs 311nDZ pairs 512 | |
| minor allele | 1 | |
| allele freq | 0.068830 | |
| var(freq) | 0.032046 (est) 0.032488 (DZ+) | 0.036133 (DZ−) |
| trait mean | | |
| unrel 0.041716 | | |
| MZ 0.034043 | | |
| DZ 0.004815 | | |
| tot 0.002781 | | |
| trait var | | |
| Tot 0.125760 | | |
| Gen | 0.039109 (0.310984) | |
| ShEnv | 0.037473 (0.297968) | |
| NShEnv | 0.049178 (0.391047) | |
| corln | 0.453460 | |

| hwTest | N(AA) | N(AB) | N(BB) | N | p | q |
|---|---|---|---|---|---|---|
| Obs: | 919 | 158 | 0 | 1077 | 0.92665 | 0.07335 |
| Exp: | 924.79 | 146.41 | 5.79 | | | |
| Delta: | −5.79 | 11.59 | −5.79 | | | |
| ChiSq: | 0.04 | 0.92 | 5.79 | | | |
| | N(AA + BB) | N(AB) | | | | |
| Obs: | 919 | 158 | | | | |
| Exp: | 930.59 | 146.41 | | | | |
| Delta: | −11.59 | 11.59 | | | | |
| ChiSq: | 0.14 | 0.92 | | | | |

| Test | Value | P-Value |
|---|---|---|
| 3-bin chisq | 6.74852 | 0.00938253 |
| 2-bin chisq | 1.06175 | 0.302816 |

Skipping because nCat is too small: 2

| | | | |
|---|---|---|---|
| unrel | 0.000000 | 0.000000 +/− 1.000000 | pval 1.000000 |
| mean | 0.065319 | 0.023164 +/− 0.044701 | pval 0.604317 |
| diff | −0.524646 | −0.186054 +/− 0.047437 | pval 0.000088 |
| diffnp | −1.134105 | −0.402184 +/− 0.116532 | pval 0.000558 |
| tot | −0.211932 | −0.075157 +/− 0.032515 | pval 0.020809 |
| strat | 0.589965 | 0.209218 +/− 0.065180 | pval 0.001328 |

### Equivalents

From the foregoing detailed description of the specific embodiments of the invention, it should be apparent that unique compositions and methods of use thereof in SNPs in known genes have been described. Although particular embodiments have been disclosed herein in detail, this has been done by way of example for purposes of illustration only, and is not intended to be limiting with respect to the scope of the appended claims which follow. In particular, it is contemplated by the inventor that various substitutions, alterations, and modifications may be made to the invention without departing from the spirit and scope of the invention as defined by the claims.

What is claimed is:

1. An isolated polypeptide comprising the mature form of amino acid sequence SEQ ID NO:2.

2. An isolated polypeptide comprising an amino acid sequence SEQ ID NO:2.

3. An isolated polypeptide comprising an amino acid sequence which is at least 95% identical to amino acid sequence SEQ ID NO:2.

4. An isolated polypeptide, wherein the polypeptide comprises an amino acid sequence comprising one or more conservative substitutions in the amino acid sequence of SEQ ID NO:2.

5. The polypeptide of claim 1 wherein said polypeptide is naturally occurring.

6. A composition comprising the polypeptide of claim 1 and a carrier.

7. A kit comprising, in one or more containers, the composition of claim 6.

8. The use of a therapeutic in the manufacture of a medicament for treating a syndrome associated with a human disease, the disease selected from a pathology associated with the polypeptide of claim 1, wherein the therapeutic comprises the polypeptide of claim 1.

9. A method for determining the presence or amount of the polypeptide of claim 1 in a sample, the method comprising:
(a) providing said sample;
(b) introducing said sample to an antibody that binds immunospecifically to the polypeptide; and
(c) determining the presence or amount of antibody bound to said polypeptide,
thereby determining the presence or amount of polypeptide in said sample.

10. A method for determining the presence of or predisposition to a disease associated with altered levels of expression of the polypeptide of claim 1 in a first mammalian subject, the method comprising:
a) measuring the level of expression of the polypeptide in a sample from the first mammalian subject; and
b) comparing the expression of said polypeptide in the sample of step (a) to the expression of the polypeptide present in a control sample from a second mammalian subject known not to have, or not to be predisposed to, said disease,
wherein an alteration in the level of expression of the polypeptide in the first subject as compared to the control sample indicates the presence of or predisposition to said disease.

11. A method of identifying an agent that binds to the polypeptide of claim 1, the method comprising:
(a) introducing said polypeptide to said agent; and
(b) determining whether said agent binds to said polypeptide.

12. The method of claim 11 wherein the agent is a cellular receptor or a downstream effector.

13. A method for identifying a potential therapeutic agent for use in treatment of a pathology, wherein the pathology is related to aberrant expression or aberrant physiological interactions of the polypeptide of claim 1, the method comprising:
(a) providing a cell expressing the polypeptide of claim 1 and having a property or function ascribable to the polypeptide;
(b) contacting the cell with a composition comprising a candidate substance; and
(c) determining whether the substance alters the property or function ascribable to the polypeptide,
whereby, if an alteration observed in the presence of the substance is not observed when the cell is contacted with a composition in the absence of the substance, the substance is identified as a potential therapeutic agent.

14. A method for screening for a modulator of activity of or of latency or predisposition to a pathology associated with the polypeptide of claim 1, said method comprising:
(a) administering a test compound to a test animal at increased risk for a pathology associated with the polypeptide of claim 1, wherein said test animal recombinantly expresses the polypeptide of claim 1;
(b) measuring the activity of said polypeptide in said test animal after administering the compound of step (a); and
(c) comparing the activity of said polypeptide in said test animal with the activity of said polypeptide in a control animal not administered said polypeptide,
wherein a change in the activity of said polypeptide in said test animal relative to said control animal indicates the test compound is a modulator activity of or latency or predisposition to, a pathology associated with the polypeptide of claim 1.

15. The method of claim 14, wherein said test animal is a recombinant test animal that expresses a test protein transgene or expresses said transgene under the control of a promoter at an increased level relative to a wild-type test animal, and wherein said promoter is not the native gene promoter of said transgene.

16. A method for modulating the activity of the polypeptide of claim 1, the method comprising contacting a cell sample expressing the polypeptide of claim 1 with a compound that binds to said polypeptide in an amount sufficient to modulate the activity of the polypeptide.

17. A method of treating or preventing a pathology associated with the polypeptide of claim 1, the method comprising administering the polypeptide of claim 1 to a subject in which such treatment or prevention is desired in an amount sufficient to treat or prevent the pathology in the subject.

18. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising the amino acid sequence of SEQ ID NO:2 or a biologically active fragment thereof.

19. An isolated nucleic acid molecule comprising a nucleic acid sequence SEQ ID NO: 1.

20. The nucleic acid molecule of claim 19, wherein the nucleic acid molecule is naturally occurring.

21. A nucleic acid molecule, wherein the nucleic acid molecule differs by a single nucleotide from a nucleic acid sequence consisting of SEQ ID NO: 1.

22. An isolated nucleic acid molecule encoding the mature form of a polypeptide having an amino acid sequence consisting of SEQ ID NO:2.

23. The nucleic acid molecule of claim 19, wherein said nucleic acid molecule hybridizes under stringent conditions to the nucleotide sequence of SEQ ID NO: 1, or a complement of said nucleotide sequence.

24. A vector comprising the nucleic acid molecule of claim 19.

25. The vector of claim 24, further comprising a promoter operably linked to said nucleic acid molecule.

26. A cell comprising the vector of claim 24.

27. An antibody that immunospecifically binds to the polypeptide of claim 1.

28. The antibody of claim 27, wherein the antibody is a monoclonal antibody.

29. The antibody of claim 27, wherein the antibody is a humanized antibody.

30. The antibody of claim 27, wherein the antibody is a fully human antibody.

31. The antibody of claim 27, wherein the dissociation constant for the binding of the polypeptide to the antibody is less than 1×10⁻⁹ M.

32. The antibody of claim 27, wherein the antibody neutralizes an activity of the polypeptide.

33. A method of treating or preventing a NOV1-associated disorder, the method comprising administering to a subject in which such treatment or prevention is desired the antibody of claim 27 in an amount sufficient to treat or prevent the pathology in the subject.

34. A method for determining the presence or amount of the nucleic acid molecule of claim 19 in a sample, the method comprising:
(a) providing said sample;
(b) introducing said sample to a probe that binds to said nucleic acid molecule; and
(c) determining the presence or amount of said probe bound to said nucleic acid molecule,
thereby determining the presence or amount of the nucleic acid molecule in said sample.

35. A method for determining the presence of or predisposition to a disease associated with altered levels of expression of the nucleic acid molecule of claim 19 in a first mammalian subject, the method comprising:
a) measuring the level of expression of the nucleic acid in a sample from the first mammalian subject; and
b) comparing the level of expression of said nucleic acid in the sample of step (a) to the level of expression of the nucleic acid present in a control sample from a second mammalian subject known not to have or not be predisposed to, the disease;
wherein an alteration in the level of expression of the nucleic acid in the first subject as compared to the control sample indicates the presence of or predisposition to the disease.

36. A method of producing the polypeptide of claim 1, the method comprising culturing a cell under conditions that lead to expression of the polypeptide, wherein said cell comprises a vector comprising an isolated nucleic acid molecule comprising a nucleic acid sequence consisting of SEQ ID NO:1.

37. The method of claim 36 wherein the cell is a bacterial cell.

38. The method of claim 36 wherein the cell is an insect cell.

39. The method of claim 36 wherein the cell is a yeast cell.

40. The method of claim 36 wherein the cell is a mammalian cell.

41. A method of producing the polypeptide of claim 2, the method comprising culturing a cell under conditions that lead to expression of the polypeptide, wherein said cell comprises a vector comprising an isolated nucleic acid molecule comprising a nucleic acid sequence consisting of SEQ ID NO:1.

42. The method of claim 41 wherein the cell is a bacterial cell.

43. The method of claim 41 wherein the cell is an insect cell.

44. The method of claim 41 wherein the cell is a yeast cell.

45. The method of claim 41 wherein the cell is a mammalian cell.

46. An isolated polynucleotide comprising a polymorphic nucleotide sequence selected from the group consisting of SEQ ID NOS:3, 5, 6, 8, 9, and 11.

47. An isolated allele-specific oligonucleotide that hybridizes to a polynucleotide at a polymorphic site encompassed therein, wherein the polynucleotide comprises a polymorphic nucleotide sequence selected from the group consisting of SEQ ID NOS: 3, 5, 6, 8, 9, and 11.

48. An isolated nucleic acid comprising a hexokinase 3-like gene comprising a polymorphism at position 1630 as defined by the positions in SEQ ID NO:3 wherein the nucleotide corresponding to position 1630 is not a thymidine.

49. The isolated nucleic acid molecule of claim 48, wherein the nucleotide at position 814 is a thymidine.

50. An isolated nucleic acid molecule comprising a sequence complementary to the isolated nucleic acid molecule of claim 48.

51. An isolated nucleic acid comprising a SIAT-1-like gene comprising a polymorphism at position 1669 as defined by the positions in SEQ ID NO:6 wherein the nucleotide corresponding to position 1669 of SEQ ID NO:6 is not a guanosine.

52. The isolated nucleic acid molecule of claim 51, wherein the nucleotide at position 1669 is an adenosine.

53. An isolated nucleic acid molecule comprising a sequence complementary to the isolated nucleic acid molecule of claim 51.

54. An isolated nucleic acid comprising a peroxin 6-like gene comprising a polymorphism at position 3462 as defined by the positions in SEQ ID NO:9 wherein the nucleotide corresponding to position 3462 of SEQ ID NO:9 is not a cytosine.

55. The isolated nucleic acid molecule of claim 54, wherein the nucleotide at position 3462 is a thymidine.

56. An isolated nucleic acid molecule comprising a sequence complementary to the isolated nucleic acid molecule of claim 54.

57. A method for detection of at least one single nucleotide polymorphism (SNP) in a human hexokinase 3-like gene, which method comprises determining a nucleotide at position 1630 in the human hexokinase 3-like gene as defined by the positions in SEQ ID NO:3, and thereby detecting absence or presence of at least one SNP.

58. A method according to claim 57 in which the single nucleotide polymorphism at position 1630 is the presence of C or T.

59. A method for detection of at least one single nucleotide polymorphism (SNP) in a human SIAT-1-like gene, which method comprises determining a nucleotide at position 1669 in the human SIAT-1-like gene as defined by the positions in SEQ ID NO:6, and thereby detecting absence or presence of at least one SNP.

60. A method according to claim 59 in which the single nucleotide polymorphism at position 1669 is the presence of G or A.

61. A method for detection of at least one single nucleotide polymorphism (SNP) in a human peroxin 6-like gene, which method comprises determining a nucleotide at position 3462 in the human peroxin 6-like gene as defined by the positions in SEQ ID NO:9, and thereby detecting absence or presence of at least one SNP.

62. A method according to claim 61 in which the single nucleotide polymorphism at position 3462 is the presence of C or T.

63. An isolated nucleic acid comprising the 5′ untranslated region of SEQ ID NO:3, 5, 6, 8, 9, or 11.

64. An allele-specific nucleic acid primer, comprising between 17-35 nucleotides which hybridizes to and detects a hexokinase 3-like gene polymorphism at position 814 in the hexokinase 3-like gene as defined by the positions in SEQ ID NO:3.

65. An allele-specific nucleic acid primer, comprising between 17-35 nucleotides which hybridizes to and detects a SIAT-1-like gene polymorphism at position 1669 in the SIAT-1-like gene as defined by the positions in SEQ ID NO:6.

66. An allele-specific nucleic acid primer, comprising between 17-35 nucleotides which hybridizes to and detects a peroxin 6-like gene polymorphism at position 3462 in the peroxin 6-like gene as defined by the positions in SEQ ID NO:9.

67. A method for determining the presence of or predisposition to a disease or pathological condition associated with a polymorphism of SEQ ID NO:3, 6, or 9, the method comprising:
a) testing a biological sample from a mammalian subject for the presence of
a polymorphism; and
b) determining the copy number of the polymorphic allele,
wherein the copy number of the polymorphic allele indicates the presence of or predisposition to said disease or pathological condition.

68. A method for identifying the carrier status of a genetic risk-altering factor associated with a polymorphism of SEQ ID NO:3, 6, or 9, the method comprising:
a) testing a biological sample from a mammalian subject for the presence of
a polymorphism; and
b) determining the copy number of the polymorphic allele,
wherein the copy number of the polymorphic allele indicates carrier status.

69. The nucleic acid sequence of claim 49, wherein the T allele is indicative of increased serum levels of bicarbonate.

70. The method of claim 67, wherein said disease or pathological condition is selected from the group consisting of respiratory and nonrespiratory alkalosis, respiratory and/or renal complications, cardiovascular disease, non-insulin dependent diabetes (Type II Diabetes), atherosclerosis, steatosis, hypertension, microvascular disease, and stroke.

71. The method of claim 68, wherein said genetic risk factor is selected from the group consisting of increased serum levels of bicarbonate, a decrease in systolic blood pressure of 0.1 standard deviation below the mean level in the sampled population, a decrease in radial peripheral maximal dp/dt of 0.1 standard deviation below the mean level in the sampled population, and decreased BMI.

72. The nucleic acid sequence of claim 52, wherein the A allele is indicative of a decrease in systolic blood pressure or a decrease in radial peripheral maximal dp/dt of 0.1 standard deviation below the mean level in the sampled population.

73. The nucleic acid sequence of claim 55, wherein the T allele is indicative of decreased BMI.

74. A method of treating a subject suffering from, at risk for, or suspected of, suffering from a pathology ascribed to the presence of a sequence polymorphism in a subject, the method comprising:
a) providing a subject suffering from a pathology associated with aberrant expression of a first nucleic acid comprising a polymorphic sequence selected from the group consisting of SEQ ID NOS:3, 5, 6, 8, 9, and 11, or its complement, and
b) administering to the subject an effective therapeutic dose of a first nucleic acid comprising the polymorphic sequence, provided that the second nucleic acid comprises the nucleotide present in the wild type allele, thereby treating said subject.

75. A method of treating a subject suffering from, at risk for, or suspected of suffering from, a pathology ascribed to the presence of a sequence polymorphism in a subject, the method comprising:
a) providing a subject suffering from, at risk for, or suspected of suffering from, a pathology associated with aberrant expression of a nucleic acid comprising a polymorphic sequence selected from the group consisting of SEQ ID NOS:3, 5, 6, 8, 9, and 11, or its complement, and
b) administering to the subject an effective dose of an oligonucleotide comprising a polymorphic sequence selected from the group consisting of SEQ ID NOS:3, 5, 6, 8, 9, and 11, or by a polynucleotide comprising a nucleotide sequence that is complementary to any one of polymorphic sequences SEQ ID NOS:3, 5, 6, 8, 9, or 11,
thereby treating said subject.

76. An oligonucleotide array, comprising one or more oligonucleotides hybridizing to a first polynucleotide at a polymorphic site encompassed therein, wherein the first polynucleotide is chosen from the group consisting of:
a) a nucleotide sequence comprising one or more polymorphic sequences selected from the group consisting of SEQ ID NOS:3, 5, 6, 8, 9, and 11;
b) a nucleotide sequence that is a fragment of any of said nucleotide sequence, provided that the fragment includes a polymorphic site in said polymorphic sequence;
c) a complementary nucleotide sequence comprising a sequence complementary to one or more polymorphic sequences selected from the group consisting of SEQ ID NOS:3, 5, 6, 8, 9, and 11; and
d) a nucleotide sequence that is a fragment of said complementary sequence, provided that the fragment includes a polymorphic site in said polymorphic sequence.

77. The array of claim 76, wherein said array comprises about 10-1000 oligonucleotides.
